(19) United States
(12) Patent Application Publication
(10) Pub. No.: US 2025/0332285 A1
(43) Pub. Date: Oct. 30, 2025

(54) ENGINEERING SUICIDE GENE APPROACHES TO IMPROVE CHEMOTHERAPEUTIC RESPONSE IN CANCER

(71) Applicant: University of Virginia Patent Foundation, Charlottesville, VA (US)

(72) Inventors: Matthew J. Lazzara; Benjamin W. Purow; William S. Hart

(73) Assignee: University of Virginia Patent Foundation, Charlottesville, VA (US)

(21) Appl. No.: 18/848,155

(22) PCT Filed: Mar. 20, 2023
(86) PCT No.: PCT/US2023/064725

§ 371 Date: Sep. 18, 2024

Related U.S. Application Data
(63) Continuation-in-part of application No. 16/669,191, filed on Oct. 30, 2019.
(60) Provisional application No. 62/752,631, filed on Oct. 30, 2018.
(60) Provisional application No. 63/321,586, filed on Mar. 18, 2022.

(51) Int. Cl. A61K 48/00; A61K 31/513; A61K 31/522; A61K 45/06; A61P 35/00; C12N 9/12; C12N 9/78; C12N 15/86

(52) U.S. Cl. CPC A61K 48/005; A61K 31/513; A61K 31/522; A61K 45/06; A61P 35/00; C12N 9/1211; C12N 9/78; C12N 15/86; C07K 2319/09; C12Y 207/01021; C12Y 207/11024; C12Y 305/04001

(57) ABSTRACT

Disclosed are compositions and methods for treating a disease and/or disorder such as cancer in a subject in need thereof. In some embodiments, the method includes administering to the subject a nucleic acid construct that includes a first nucleic acid sequence having a promoter operably linked to each of a second nucleic acid sequence encoding a therapeutic polypeptide, and a third nucleic acid sequence encoding a peptide domain that is stabilized when phosphorylated by stress-activated kinase activity in a target cell and/or tissue. In some embodiments, the target cell and/or tissue is a cell and/or tissue undergoing a stress response. In some embodiments, the kinase activity is stress-activated kinase activity mediated by p38/MAP14 kinase activity, INK activity, or both.

### CROSS REFERENCE TO RELATED APPLICATIONS

The presently disclosed subject matter claims priority to and the benefit of U.S. Provisional Patent Application Ser. No. 63/321,586, filed Mar. 18, 2022. This application is also a continuation-in-part of U.S. patent application Ser. No. 16/669,191, filed on Oct. 30, 2019, which claims benefit of U.S. Provisional Patent Application Ser. No. 62/752,631, filed Oct. 30, 2018. The disclosure of each of these applications is herein incorporated by reference in its entirety.

### GOVERNMENT SUPPORT

This invention was made with government support under Grant No. CA252576 awarded by the National Institutes of Health. The government has certain rights in the invention.

### REFERENCE TO SEQUENCE LISTING XML

The Sequence Listing XML associated with the instant disclosure has been electronically submitted to the United States Patent and Trademark Office via the Patent Center as a 75,633 byte UTF-8-encoded XML file created on Mar. 20, 2023 and entitled “3062_181_PCT. xml”. The Sequence Listing submitted via Patent Center is incorporated herein by reference in its entirety.

### TECHNICAL FIELD

The presently disclosed subject matter relates to compositions and methods for treating cancer in a subject in need thereof. In some embodiments, the presently disclosed subject matter relates to compositions and methods employing a stress-activated kinase-stabilized suicide gene encoding a protein that converts a prodrug into a toxic product.

### BACKGROUND

Many cancers involve aberrant signaling through the Ras/Raf/MEK/ERK pathway. While pharmacological inhibitors exist to target some of the nodes in this signaling cascade, cancer cells can leverage multiple opportunities to develop resistance to those inhibitors, most often in ways that lead to maintenance of extracellular regulated kinase (ERK) signaling. Because maintenance of ERK signaling in cancer cells can be a potent driver of cancer cell survival, there is a need for new and orthogonal mechanisms to target this signaling pathway for the treatment of various types of cancer.

For example, approximately 14,000 new cases of glioblastoma multiforme (GBM) are diagnosed in the United States each year. Median survival time for these patients is a dismal 18 months due to GBM resistance to current modalities of chemoradiation and a general inability to resect surgically tumor cells that diffusely spread. It is estimated that as many as 90% of all GBM tumors display dysregulation of receptor-mediated signaling processes that drive Ras/ERK signaling. Thus, the vast majority of the GBM patient population may benefit from new approaches to target Ras/ERK signaling as would patients suffering from other types of cancer.

In addition to the ERK pathways, c-Jun N-terminal kinases (JNKs) and p38/MAPK14 isoforms (p38α, p38β, p38γ, and p38δ) have been shown to be implicated in inflammation and other diseases and disorders including cancer (Huang et al., 2010; Garcia-Hernandez et al., 2021). JNKs (also referred to as stress-activated protein kinases (SAPKs)) are activated by a MAPK3 cascade via MEK4 and MEK7 (Manzoor et al., 2012). JNKs regulate cell proliferation and apoptosis by activating various targets, including the AP-1 transcription factor. AP-1 is activated by cytokines, stress, growth factors, and infections, among other stimuli, and is involved in managing proliferation, differentiation, and apoptosis. MAPKs have been shown to play important roles in chronic inflammation and inflammation-associated cancer development.

Taking advantage of the biological activities of SAPKs and MAPKs in cells and tissues that express these polypeptides can thus provide a way of treating diseases and disorders including cancer.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, the presently disclosed subject matter relates to nucleic acid constructs. In some embodiments, a nucleic acid construct of the presently disclosed subject matter comprises, consists essentially of, or consists of a first nucleic acid sequence comprising a promoter operably linked to each of a second nucleic acid sequence encoding a therapeutic polypeptide and a third nucleic acid sequence encoding a peptide domain that is stabilized when phosphorylated by kinase activity in a target cell and/or tissue, wherein the target cell and/or is a cell and/or undergoing a stress response and the kinase activity is associated with the target cell and/or undergoing a stress response, optionally wherein the kinase is a p38 MAP kinase and/or a c-Jun N-terminal kinase (INK). In some embodiments, the nucleic acid construct further comprises a fourth nucleic acid sequence encoding a nuclear localization sequence (NLS) operably linked to a promoter.

In some embodiments of the presently disclosed subject matter, the first nucleic acid sequence comprises a promoter operably linked to a second nucleic acid sequence encoding a fusion protein comprising the therapeutic polypeptide and the peptide domain that is stabilized when phosphorylated by p38 MAP kinase activity and/or INK activity.

In some embodiments of the presently disclosed subject matter, the second nucleic acid sequence encoding the fusion protein comprises: (a) a first coding sequence encoding the therapeutic polypeptide; (b) a second coding sequence encoding an NLS; and (c) a third coding sequence encoding the peptide domain that is stabilized when phosphorylated by p38 MAP kinase activity and/or JNK activity.

In some embodiments of the presently disclosed subject matter, the therapeutic polypeptide comprises a Herpes simplex virus thymidine kinase (HSVtk) polypeptide or a yeast cytosine deaminase polypeptide. In some embodiments, the HSVtk polypeptide comprises an amino acid sequence that is at least 95%, 96%, 97%, 98%, or 99% identical to SEQ ID NO: 1 or a fragment thereof, or is 100% identical to SEQ ID NO: 1 or a fragment thereof. In some embodiments, the amino acid sequence comprises at least one modification selected from the group consisting of an amino acid deletion, an amino acid addition, an amino acid substitution, and combinations thereof as compared to SEQ ID NO: 1.

In some embodiments of the presently disclosed subject matter, the yeast cytosine deaminase polypeptide comprises an amino acid sequence that is at least 95%, 96%, 97%, 98%, or 99% identical to SEQ ID NO: 5 or a fragment thereof, or is 100% identical to SEQ ID NO: 5 or a fragment thereof. In some embodiments, the amino acid sequence comprises at least one modification selected from the group consisting of an amino acid deletion, an amino acid addition, an amino acid substitution, and combinations thereof as compared to SEQ ID NO: 5.

In some embodiments of the presently disclosed subject matter, the peptide domain that is stabilized when phosphorylated by p38 MAP kinase activity and/or JNK activity in a target cell and/or tissue comprises a peptide domain as set forth in any of FIGS. 15-20, or a fragment thereof, or a substantially homologous amino acid sequence thereto, or a substantially homologous amino acid sequence to a fragment thereof. In some embodiments, the peptide domain comprises amino acids 266-282 of SEQ ID NO. 11, amino acids 444-462 of SEQ ID NO. 11, amino acids 2-101 of SEQ ID NO. 13, amino acids 50-101 of SEQ ID NO. 13, amino acids 122-140 of SEQ ID NO. 16, amino acids 163-271 of SEQ ID NO. 18, amino acids 180-196 of SEQ ID NO. 18, or amino acids 251-270 of SEQ ID NO. 18.

In some embodiments of the presently disclosed subject matter, the nucleic acid construct is present within a vector, optionally a viral vector. In some embodiments, the nucleic acid construct, optionally the vector, is disposed in a pharmaceutically acceptable diluent or vehicle.

In some embodiments, the presently disclosed subject matter relates to kits comprising, consisting essentially of, or consisting of the nucleic acid constructs as disclosed herein and at least one reagent and/or device for introducing the nucleic acid constructs into cells, tissues, and/or subjects. In some embodiments, a kit of the presently disclosed subject matter further comprises instructions for introducing the nucleic acid constructs into cells, tissues, and/or subjects.

In some embodiments, the presently disclosed subject matter also relates to methods for treating diseases and/or disorders in subjects in need thereof. In some embodiments, the methods comprise, consist essentially of, or consist of administering to the subject a nucleic acid construct comprising a first nucleic acid sequence comprising a promoter operably linked to each of a second nucleic acid sequence encoding a therapeutic polypeptide and a third nucleic acid sequence encoding a peptide domain that is stabilized when phosphorylated by kinase activity in a target cell and/or, wherein the target cell and/or is a cell and/or undergoing a stress response and the kinase activity is associated with the target cell and/or undergoing a stress response, optionally wherein the kinase is a p38 MAP kinase and/or a c-Jun N-terminal kinase (INK); and administering to the subject a prodrug that is converted by the therapeutic polypeptide to an active agent.

In some embodiments, the amino acid sequence of the peptide domain comprises at least one modification selected from the group consisting of an amino acid deletion, an amino acid addition, an amino acid substitution, and combinations thereof as compared to SEQ ID NOs: 1, 4, 5, 7-20, 22, 24, 26, 28, 30, 32, or 34. In some embodiments, the nucleic acid construct further comprises a fourth nucleic acid sequence encoding a nuclear localization sequence (NLS) operably linked to a promoter. In some embodiments, the second nucleic acid sequence encodes a fusion protein comprising the therapeutic polypeptide and the peptide domain that is stabilized when phosphorylated by p38 MAP kinase activity and/or and/or JNK activity. In some embodiments, the second nucleic acid sequence encoding the fusion protein comprises: (a) a nucleic acid sequence encoding the therapeutic polypeptide; (b) a nucleic acid sequence encoding an NLS; and (c) a nucleic acid sequence encoding the peptide domain that is stabilized when phosphorylated by kinase activity. In some embodiments, the therapeutic polypeptide comprises a Herpes simplex virus thymidine kinase (HSVtk) polypeptide or a yeast cytosine deaminase polypeptide. In some embodiments, the HSVtk polypeptide comprises an amino acid sequence that is at least 95%, 96%, 97%, 98%, 99% identical to SEQ ID NO: 1 or a fragment thereof or is 100% identical to SEQ ID NO: 1 or a fragment thereof.

In some embodiments of the presently disclosed methods, the amino acid sequence comprises at least one modification selected from the group consisting of an amino acid deletion, an amino acid addition, an amino acid substitution, and combinations thereof as compared to SEQ ID NO: 1. In some embodiments, the yeast cytosine deaminase polypeptide comprises an amino acid sequence that is at least 95%, 96%, 97%, 98%, 99% identical to SEQ ID NO: 5 or a fragment thereof or is 100% identical to SEQ ID NO: 5 or a fragment thereof. In some embodiments, the amino acid sequence comprises at least one modification selected from the group consisting of an amino acid deletion, an amino acid addition, an amino acid substitution, and combinations thereof as compared to SEQ ID NO: 5.

In some embodiments of the presently disclosed methods, the peptide domain that is stabilized when phosphorylated by p38 MAP kinase activity and/or INK activity in a target cell and/or a target tissue comprises, consists essentially of, or consists of a peptide domain as set forth in any one of FIGS. 15-20, or fragment thereof, or a substantially homologous amino acid sequence thereto, or a substantially homologous amino acid sequence to a fragment thereof.

In some embodiments, the nucleic acid construct is present in a vector, optionally a viral vector.

In some embodiments of the presently disclosed methods, the nucleic acid construct and/or the prodrug is/are administered in a pharmaceutically acceptable diluent or vehicle.

In some embodiments of the presently disclosed methods, the prodrug is selected from the group consisting of ganciclovir, acyclovir, and 5-fluorocytosine.

In some embodiments of the presently disclosed methods, the disease or disorder is cancer. In some embodiments, the cancer is glioblastoma.

In some embodiments, the presently disclosed methods further comprise administering an additional treatment and/or therapeutic agent to the subject. In some embodiments, the additional treatment and/or therapeutic agent is an anti-cancer drug, radiation, surgery, or a combination thereof.

In some embodiments, the amino acid sequence of a peptide domain comprises at least one modification selected from the group consisting of an amino acid deletion, an amino acid addition, an amino acid substitution, and combinations thereof as compared to SEQ ID NOs: 1, 4, 5, 7-20, 22, 24, 26, 28, 30, 32, or 34.

Accordingly, it is an object of the presently disclosed subject matter to provide compositions and methods for treating cancer. This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Figures, and EXAMPLES.

### BRIEF DESCRIPTION OF THE FIGURES

The presently disclosed subject matter can be better understood by referring to the following Figures. The components in the Figures are not necessarily to scale, emphasis instead being placed upon illustrating the principles of the presently disclosed subject matter (often schematically). In the Figures, like reference numerals designate corresponding parts throughout the different views. A further understanding of the presently disclosed subject matter can be obtained by reference to an embodiment set forth in the illustrations of the accompanying Figures. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the Figures and the following description. The Figures are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following Figures in which:

FIGS. 1A and 1B are schematic representations of a fluorescence based ERK activity FIRE reporter described by Albeck et al., 2013 (FIG. 1A) and an embodiment of an ERK activity suicide gene, HSVtk-FIRE, in accordance with the presently disclosed subject matter (FIG. 1). NLS: nuclear localization sequence. mVenus: coding sequence for the mVenus bright yellow monomeric fluorescent protein. Fra-1 163-271: nucleotides 10 encoding amino acids 163-271 of SEQ ID NO:4, which encode an exemplary PEST domain of the presently disclosed subject matter. FIRE: Fra-1 based Integrative Reporter. The two circled “P” in the Fra-1 domain represents phosphorylatable serines present these domains that when phosphorylated result in stabilization of the mVenus domain or the HSVtk domain.

FIG. 2A is a Western blot analysis of U87MG cells transduced with HSVtk-FIRE and treated with or without the MEK inhibitor CI-1040 (i. e., 2-(2-chloro-4-iodoanilino)-N-(cyclopropylmethoxy)-3,4-difluorobenzamide; CAS Number 212631-79-3). CI-1040 (also called PD184352) is an orally active, highly specific, small-molecule inhibitor of one of the key components of this pathway (MEK1/MEK2), and thereby effectively blocks the phosphorylation of ERK and continued signal transduction through this pathway. Fra-1 is a transcription factor that is phosphorylated by ERK.

FIG. 2B is a graph of flow cytometry-based measurements of U87MG cells expressing HSVtk-FIRE treated with ganciclovir (GCV) and/or CI-1040 at 0.5 M (gray bars) or 5 M (black bars). White bars are a DMSO negative control. Errors bars are standard error of the mean (SEM) of three replicates unless otherwise indicated.

FIG. 3A is a schematic overview of a prodrug-based suicide gene strategy. As depicted, a virus encoding a suicide gene is introduced into a host cell, where it is converted to a gene product that can convert a pro-drug (squares) into a cytotoxic product (stars).

FIG. 3B is a reaction scheme showing a particular mechanism at play in the HSVtk suicide gene approach, involving the conversion of GCV to GCV monophosphate, a toxic reagent that produces DNA damage (double strand breaks) that can lead to cell death, by HSVtk.

FIG. 4A is a Western blot analysis of U87MG cells transduced with empty vector (EV), HSVtk-d2 control vector (an ERK-independent PEST domain (d2) counterpart that is not phosphorylated), or HSVtk-FIRE in the presence (+) or absence of the MEK inhibitor CI-1040. In the top panel, CI-1040 treatment is shown to inhibit the phosphorylation of Fra-1 and ERK. The bottom panel shows a loading control.

FIG. 4B is a series of bar graphs showing cell death measurements by flow cytometry (ToPro3 permeability) for the same cell lines treated with DMSO (white bars) or CI-1040 (black bars). *: p<0.05. n. s.: not significant (p>0.05). Errors bars are ±standard error of the mean (SEM) of three replicates unless otherwise indicated.

FIG. 4C is immunofluorescence imaging showing ERK-regulated DNA damage response in cells expressing HSVtk-FIRE. The DNA damage was caused by GCV conversion by HSVtk to a monophosphoylrated form. ERK regulated the stability of the HSVtk fusion cassette. As such, ERK inhibition mitigated the ability of GCV to drive DNA damage because it resulted in reduced HSVtk expression.

FIG. 5A is a schematic reaction scheme showing a ERK-stabilized suicide gene product, which converts the prodrug ganciclovir (GCV) into a toxic product through the expression of the Herpes simplex virus thymidine kinase (HSVtk) protein, where the HSVtk protein has been fused to a nuclear localization sequence (NLS) and a peptide domain (Fra1 domain) that is stabilized when phosphorylated by active ERK, which is preferentially shuttled to the cell nucleus.

FIG. 5B is a schematic of an embodiment of an ERK activity suicide gene, HSVtk-FTRE, in accordance with the presently disclosed subject matter. 5′LTR: 5′ long terminal repeat. Ψ: packaging sequence. gag: retroviral gag promoter. NLS: nuclear localization sequence. HSVtk: Herpes simplex virus thymidine kinase coding sequence. FRA1 163-271: nucleotides encoding amino acids 163-271 of SEQ ID NO:4, which is referred to herein as a PEST domain. IRES: internal ribosome entry site. GFP: green fluorescent protein coding sequence. 3′LTR: 3′ long terminal repeat.

FIG. 6A is a schematic of an embodiment of an ERK activity suicide gene cassette, HSVtk-FIRE, in accordance with the presently disclosed subject matter. CMV: cytomegalovirus promoter. NLS: nuclear localization sequence. HSVtk: Herpes simplex virus thymidine kinase coding sequence. FRA1 163-271: nucleotides encoding amino acids 163-271 of SEQ ID NO:4, which is referred to herein as a PEST domain. IRES: internal ribosome entry site. eGFP: enhanced green fluorescent protein coding sequence.

FIG. 6B is immunofluorescence imaging showing subcellular localization of the NLS-HSVtk-FIRE construct using HSVtk expression constructs which contain an N-terminal Flag-tag. These constructs demonstrate that the NLS and FIRE elements of the construct are causing the anticipated effects on localization.

FIG. 7A is Western blot (leftpanel) and bar graph (right panel) showing decreased ERK activation, due to treatment with the MEK inhibitor CI-1040 (black bars in right panel), causes reduced cell death in response to GCV in U87MG glioblastoma cells stably expressing the variant III mutant of the EGFR (U87MG+EGFRvIII; see Day et al., 2021) expressing HSVtk-FIRE (NLS-HSVtk-FIRE), compared to an ERK-independent PEST domain (d2) counterpart (NLS-HSVtk-d2). *: p<0.05. n. s.: not significant (p>0.05). Errors bars are ±standard error of the mean (SEM) of three replicates unless otherwise indicated.

FIG. 7B is Western blot (left panel) and bar graph (right panel) showing similar results as seen in FIG. 7A, but in G88 glioma stem cells treated with the MEK inhibitor trametinib (N-[3-[3-cyclopropyl-5-(2-fluoro-4-iodoanilino)-6,8-dimethyl-2,4,7-trioxopyrido[4,3-d]pyrimidin-1-yl]phenyl]acetamide). *: p<0.05. Errors bars are standard error of the mean (SEM) of three replicates unless otherwise indicated.

FIG. 8 is a Western blot (top left panel) and a series of bar graphs (other panels) showing normal human pancreatic ductal epithelial cells (HPDE) transduced with oncogenic KRAS (KRAS^{G12}V) were characterized by increased levels ERK and NLS-HSVtk-FIRE phosphorylation when compared to matched control cells (EV). The HPDE cells expressing KRAS^{G}12V have greater levels of DNA damage (pH2A. X) in response to GCV (black bars) as compared to DMSO controls (white bars). *: p<0.05. n. s.: Errors bars are ±standard error of the mean (SEM) of three replicates unless otherwise indicated.

FIG. 9A is a series of bar graphs (left and right panels) and a Western blot (middle panel) showing a comparison of NLS-HSVtk-FIRE against HSVtk lacking a PEST domain in U87MG+EGFRvIII cells. U87MG cells equally transduced with nuclear-localized HSVtk responded greater to GCV when the FTRE PEST domain was included. In the left panel, white bars correspond to HSVtk lacking a PEST domain (NLS-HSVtk) and black bars correspond to HSVtk with a PEST domain (NLS-HSVtk-FIRE). In the right panel, white bars correspond to a DMSO negative control and black bars correspond to treatment with GCV. *: p<0.05. n. s.: Errors bars are ±standard error of the mean (SEM) of three replicates unless otherwise indicated.

FIG. 9B is a bar graph (left panel) and a Western blot (right panel) showing a comparison of HSVtk-FIRE (NLS-HSVtk-FIRE) against HSVtk lacking a PEST domain (NLS-HSVtk) similar to the results shown in FIG. 9A, but in G88 cells. In the Western blot, pFRA1 is phosphorylated FRA1, pERK is phosphorylated ERK, pH2A. X is phosphorylated histone H2. AX, a measure of DNA damage; PARP is Poly (ADP-ribose) polymerase; and GAPDH is glyceraldehyde 3-phosphate dehydrogenase, which serves as a loading control. *: p<0.05. n. s.: Errors bars are ±standard error of the mean (SEM) of three replicates unless otherwise indicated.

FIGS. 10A-10C are a set of graphs and images showing U87MG cells equally transduced with nuclear-localized HSVtk respond greater to GCV, in a subcutaneous mouse model, when the FTRE PEST domain is included. In FIG. 10A, subcutaneous xenograft tumor volumes are shown for subcutaneous xenograft tumors formed in mice using GFP-sorted U87MG cells. After 6 days, mice were treated for the indicated times with 50 mg/kg GCV or vehicle. Tumor growth curves for EV- (dashed lines) and vehicle-treated (solid lines) groups are shown. Statistical comparisons between vehicle- and GCV-treated groups were made by Student's t-test, and * indicates p<0.05. FIG. 10B shows representative examples of excised tumors. FIG. 10C shows Statistical comparisons from one-way ANOVA for HSVtk (triangles), HSVtk lacking a PEST domain (NLS-HSVtk; circles), and HSVtk-FIRE (NLS-HSVtk-FIRE; diamonds). Errors bars are ±standard error of the mean (SEM) of three replicates unless otherwise indicated.

FIGS. 11A-11C are a schematic (FIG. 11A) and a set of images showing intracranial delivery of viral particles. In FIG. 11B, the scale bar indicates 1 mm. As shown in FIG. 11C, DNA damage resulted in response to GCV in the cells that were successfully transduced (i.e., those that expressed GFP).

FIG. 12A is a reaction scheme showing a ERK-stabilized suicide gene product, which converts the prodrug 5-fluorocytosine (5-FC) into a toxic product through the expression of the yeast cytosine deaminase (yCD) protein, where the yCD protein has been fused to a nuclear localization sequence (NLS) and a peptide domain (Fra1 domain) that is stabilized when phosphorylated by active ERK, which is preferentially shuttled to the cell nucleus.

FIG. 12B is a schematic of an embodiment of an ERK activity suicide gene, yCD-FTRE, in accordance with the presently disclosed subject matter. Abbreviations are as in FIG. 5B, with yCD referring to a yeast cytosine deaminase protein coding sequence.

FIG. 13 is a Western blot showing that yCD expression can be stabilized by growth factors that drive ERK activity and antagonized by MEK inhibition.

FIGS. 14A and 14B are graphs showing that ERK activity promotes cell killing in response to 5-fluorocytosine (5-FC) in cells expressing yCD-FIRE (black bars in FIG. 14A), optionally in the presence of EGF and FGF (gray bars in FIG. 14A).

FIG. 15 presents the nucleotide and amino acid sequences of a representative CHOP construct employed with p38 MAP kinase as disclosed herein. In FIG. 15, the top strand of the nucleotide sequence corresponds to SEQ ID NO: 21, and the botton strand of the nucleotide sequence corresponds to the reverse complement of SEQ ID NO: 21. The amino acid sequence shown corresponds to SEQ ID NO: 22. Also shown are certain features of the cassette including particular restriction endonuclease recognition sequences, amino acids that are part of the HSVtk polypeptide (HSV-TK), and CHOP amino acids 2-101, which include three copies of the PEST domain from the N-terminus of CHOP. The asterisks identify phosphorylatable serines in the PEST domains. Also identified by the boxed sequences is a ubiquitin ligase binding site.

FIG. 16 presents the nucleotide and amino acid sequences of a representative MEF2A/FRA1 construct as disclosed herein. In FIG. 16, the top strand of the nucleotide sequence corresponds to SEQ ID NO: 23, and the botton strand of the nucleotide sequence corresponds to the reverse complement of SEQ ID NO: 23. The amino acid sequence shown corresponds to SEQ ID NO: 24. Also shown are certain features of the cassette including particular restriction endonuclease recognition sequences, amino acids that are part of the HSVtk polypeptide (HSV-TK), a p38 targeting region derived from MEF2A, and three PEST domains, the first and third of which are from Fra-1 and the middle of which is from MEF2A. The asterisk identifies a phosphorylatable serine in the MEF2A PEST domain. Also identified by the boxed sequences is a ubiquitin ligase binding site. THe boxed amino acids are from the Fra-1 PEST domain and surrounding amino acids.

FIG. 17 presents the nucleotide and amino acid sequences of a representative CDKN1A(p21)/FRA1 construct as disclosed herein. In FIG. 17, the top strand of the nucleotide sequence corresponds to SEQ ID NO: 25, and the botton strand of the nucleotide sequence corresponds to the reverse complement of SEQ ID NO: 25. The amino acid sequence shown corresponds to SEQ ID NO: 26. Also shown are certain features of the cassette including particular restriction endonuclease recognition sequences, amino acids that are part of the HSVtk polypeptide (HSV-TK), and three PEST domains, the first and third of which are from Fra-1 and the middle of which is from p21. The asterisk identifies a phosphorylatable serine in the p21 PEST domain. Also identified by the boxed sequences is a ubiquitin ligase binding site. THe boxed amino acids are from the Fra-1 PEST domain and surrounding amino acids.

FIG. 18 is a schematic representation comparing exemplary ERK-dependent and p38-dependent cassettes. As shown, in an exemplary ERK-dependent cassette, Fra-1 amino acids 163-271 are emnployed as part of a 2×PEST and ERK-binding region domain. In the exemplary p38-dependent designs, amino acids 2-101 of CHOP includes three PEST domains (3×PEST), whereas amino acids 50-101 provides two PEST domains (2×PEST). In alternative embodiments, MEF2A amino acids 266-282 include a p38 targeting domain, which can be fued to amino acids 180-196 of Fra-1 as a first PEST domain, amino acids 444-462 of MEF2A as a second PEST domain, and amino acids 251-270 of Fra-1 as a third PEST domain. Finally, as another embodiment, amino acids 180-196 of Fra-1 as a first PEST domain, amino acids 122-140 of p21 as a second PEST domain, and amino acids 251-270 of Fra-1 as a third PEST domain.

FIGS. 19A and 19B show schematic depictions of uses of ERK-dependent and p38-dependent cassettes in suicide gene methodologies. In each Figure, the presence of sufficient levels of kinase activity (ERK in FIG. 19A and p38 in FIG. 19B) results in phosphorylation of the PEST domains, which results in phosphorylation of the prodrug (e.g., GCV) to a toxic metabolite (e.g., GCV monophosphate), which leads to DNA damage and cell death.

FIG. 20 is presents various sequences (SEQ ID NOs: 11-18) that can be employed in the compositions of the presently disclosed subject matter. In FIG. 20, double underlined amino acids are from MEF2A, italicized amino acids are from CHOP (with the underlined amino acids being used in both the full length and shortened CHOP-PEST embodiments), dashed underlined amino acids are from p21, and bolded amino acids are from Fra-1.

FIG. 21 is a schematic showing a basic suicide gene therapy approach in which ganciclovir (GCV) is converted to GCV monophosphate, which is toxic to cells.

FIG. 22 is a schematic showing a suicide gene therapy approach that employs a suicide gene design that includes a PEST domain that, upon phosphorylation, stabilizes the HSVtk cassette resulting in phosphorylation of GCV and cell death. In some embodiments, the PEST domain is an FRA1-PEST domain that can be stabilized by ERK kinase activity. In some embodiments, the PEST domain part of a p38 target that can be stabilized by p38 kinase activity. It is noted that in FIG. 22, the p38 target is an exemplary stress-activated kinase, and other stress-activated kinases including but not limited to JNK can be similarly employed in the compositions and methods of the presently disclosed subject matter.

FIG. 23 shows the results of exemplary experiments validating HSVtk fusion proteins with stability that is dependent on stress-related kinase (e. g., p38 or INK) phosphorylation. In some embodiments, a PEST domain is selected for use in the compositions and methods of the presently disclosed subject matter by virtue of having a high PEST score (PEST=proline, glutamic acid, serine, threonine) and/or being derived from a protein that is primarily phosphorylated by the stress-related kinase (e. g., p38 or INK) and that in some embodiments has at least one phosphorylatable serine in the PEST domain.

FIG. 24 is a series of Western blots shows that MEF2A PEST was stabilized in part by p38 phosphorylation.

FIGS. 25A-25H show the results of exemplary experimental examples with p38 MAP kinase embodiments as disclosed herein. FIG. 25A relates to exemplary p38-targetable PEST domains. The four designs tested included peptide sequences from the MAPK targets CHOP, MEF2A, p21, and FRA1. Schematics of each design are shown, with the predicted PEST domains and relative position of serine residues demonstrated in the literature to be phosphorylated by p38 (circled “P”). CHOP sequences shown were taken from proteins primarily phosphorylated by p38, while domains shown with “FRA1” were taken from the original FRA1-PEST design. The ERK recognition sequence was removed from these designs to improve specificity toward p38. FIG. 25B is a series of immunofluorescence microscopy images demonstrating nuclear localization of FLAG signal and varying baseline abundance. U87 cells (differentiated glioblastoma cell line) were transduced with retrovirus carrying each of the four suicide genes. Nuclear-localized FLAG was only present in cells with high GFP expression and relatively high nuclear p-p38 (solid white arrows). In cells with insufficient GFP or low levels of nuclear p-p38, FLAG abundance was similarly reduced (white-outlined arrows), indicating the importance of both gene transcription and p38 phosphorylation on HSVtk abundance. Further, baseline FLAG abundance was dependent on the design of each PEST domain. Scale bars=100 m. FIGS. 25C and 25D is a Western blot (FIG. 25C) and bar graph (FIG. 25D) of G816 cells (glioma-initiating cell line) expressing each of the four HSVtk constructs grown in 21% or 1% oxygen (hypoxia), with or without SB203580 (p38 inhibitor, 10 μM) for two days. Hypoxic induction of p38 phosphorylation promoted protein stabilization, which was abrogated with the addition of a p38 inhibitor to varying degrees for each construct. In FIG. 25D, the left bar of each triplet corresponds to a negative control, the middle bar corresponds to hypoxia, and the right bar corresponds to hypoxia+p38 inhibitor (p38i). FIGS. 25E and 25F present a Western blot and a graph showing that accumulation of FLAG-NLS-HSVtk-MEF2A occurred on a time scale of days following DNA damage. G816-FLAG-NLS-HSVtk-MEF2A cells were treated with DMSO (vehicle), GCV (50 μM), or TMZ (500 μM) for up to 48 hours. Both GCV and TMZ treatment increased FLAG abundance after 48 hours of treatment, relative to vehicle-treated samples. Interestingly, FLAG abundance did increase in the vehicle-treated condition, which could be a result of increasing cellular confluence or the use of FGFb in the cell culture medium, both of which can promote p38 activity. FIGS. 25G and 25H show the results of experiments demonstrating that p38 signaling did not fully explain FLAG-NLS-HSVtk-MEF2A abundance, but ERK was not responsible for stabilization with respect to this particular construct. With respect to FIG. 25G, G816-FLAG-NLS-HSVtk-MEF2A cells were transduced with a control siRNA or an siRNA against p38 using Lipofectamine RNAiMAX. Cells were then treated with no inhibitor or SB203580 (p38 inhibitor, 10 μM) and DMSO (vehicle), TMZ, or TNFα (tumor necrosis factor α) for two days. In the cases of DMSO- and TNFα-treated cells, knockdown of p38 reduced FLAG abundance, indicating that p38 was necessary to stabilize the protein in these contexts. However, knockdown of p38 in the TMZ-treated cells increased FLAG abundance, indicating another kinase might have been involved in PEST phosphorylation. With respect to FIG. 25H, G816-FLAG-NLS-HSVtk-MEF2A cells were treated with DMSO (vehicle), SB203580 (p38 inhibitor, 10 PM), or trametinib (MEK inhibitor, 50 nM), in addition to TNFα, GCV, or TMZ for two days. FLAG expression was increased by the p38-inducing treatments TNFα, GCV, and TMZ, and was attenuated in some cases by p38 inhibition. Interestingly, MEK inhibition also promoted PEST stabilization, indicating that there might have been a compensatory MAPK signaling mechanism at work, possibly through INK.

FIGS. 26A-26C are Western blot analyses of experiments showing that temozolomide (TMZ) and hypoxia-driven stabilization of the PEST domains were abrogated by p38 inhibition, but not by p38 knockdown. G816-FLAG-NLS-HSVtk-PEST were grown in hypoxia (1% oxygen; FIG. 26A) for three days and treated with SB203580 (p38i, 10 μM), temozolomide (TMZ, 100 M; FIG. 26B), or transfected with control or p38 siRNA (FIG. 26C), then treated with temozolomide (TMZ, 100 μM) for two days.

FIGS. 27A-27C are Western blot analyses of experiments showing that p38 inhibition resulted in PEST stabilization in some contexts. In FIG. 27A, G816 and U87 cells expressing FLAG-NLS-HSVtk-CHOP were treated with temozolomide (TMZ, 100 μM) and SB203580 (p38i, 10 μM) for three days. In FIG. 27\B, U87-FLAG-NLS-HSVtk-CHOP cells were treated with temozolomide (TMZ, 100 μM), SB203580 (p38i, 10 μM), or JNK-IN-8 (JNKi, 2 μM) alone or in combination for three days. In FIG. 27CC, U87-FLAG-NLS-HSVtk-MEF2 cells were treated with trametinib (MEKi, 40 nM) or JNK-IN-8 (JNKi, 2 μM) for 24 hours. As shown, the addition of a JNK inhibitor reduced FLAG expression, indicating the the suicide gene products might also be regulated by JNK.

FIGS. 28-31 show the nucleotide and amino acid sequences of various embodiments of the presently disclosed subject matter along with certain features therein. FIG. 28 depicts exemplary CHOP construct pMSCV-FLAG-NLS-HSVtk-CHOP-IRES-GFP-II. The top strand of the nucleotide sequence corresponds to SEQ ID NO: 27 and the amino acid depicted is SEQ ID NO: 28. FIG. 29 depicts the exemplary CHOP construct pMSCV-FLAG-NLS-HSVtk-CHOP-IRES-GFP-II. The top strand of the nucleotide sequence corresponds to SEQ ID NO: 29 and the amino acid depicted is SEQ ID NO: 30. FIG. 30 depicts the exemplary chop construct pMSCV-FLAG-NLS-HSVtk-chop-IRES-GFP-II. The top strand of the nucleotide sequence corresponds to SEQ ID NO: 31 and the amino acid depicted is SEQ ID NO: 32. FIG. 31 depicts the exemplary chop construct pMSCV-FLAG-NLS-HSVtk-chop-IRES-GFP-II. The top strand of the nucleotide sequence corresponds to SEQ ID NO: 33 and the amino acid depicted is SEQ ID NO: 34.

### BRIEF DESCRIPTION OF THE SEQUENCE LISTING

SEQ ID NO: 1 is an amino acid sequence of an exemplary human alphaherpesvirus 1 thymidine kinase polypeptide. It corresponds to Accession No. AAA45811.1 of the GENBANK® biosequence database.

SEQ ID NO: 2 is a nucleotide sequence encoding an exemplary human alphaherpesvirus 1 (isolate S4.25) thymidine kinase (UL23) polypeptide. It corresponds to Accession No. MK896152.1 of the GENBANK® biosequence database.

SEQ ID NO: 3 is a nucleotide encoding an exemplary human FOSL1/FRA1 (FOS-like antigen 1/FOS-related antigen) polypeptide. It corresponds to nucleotides 5-1530 of Accession No. NM_005438.5 of the GENBANK® biosequence database.

SEQ ID NO: 4 is an amino acid sequence of an examplery human FIRE polypeptide. It corresponds to Accession No. NP_005429.1 of the GENBANK® biosequence database.

SEQ ID NO: 5 is an amino acid sequence of an exemplary yeast cytosine deaminase polypeptide. It corresponds to Accession No. NP_015387.1 of the GENBANK® biosequence database.

SEQ ID NO: 6 is an exemplary nucleotide sequence encoding a yeast cytosine deaminase polypeptide. It corresponds to Accession No. NM_001184159.1 of the GENBANK® biosequence database.

SEQ ID NOs: 7-10 are amino acid sequences of exemplary nuclear localization signals (NLS) that can be employed in the nucleic acids of the presently disclosed subject matter.

SEQ ID NO: 11 is an amino acid sequence of an exemplary MEF2A polypeptide. It corresponds to Accession No. NP_001339546.1 of the GENBANK® biosequence database.

SEQ ID NO: 12 is an amino acid sequence of an exemplary MEF2A design (C-terminally fused to HSVtk) construct. Amino acids 8-24 and 59-77 are from MEF2A and amino acids 42-58 and 78-97 are from Fra-1.

SEQ ID NO: 13 is an amino acid sequence of an exemplary CHOP (C/EBP transcription factor; DDIT3) sequence. It corresponds to amino acids 294-462 of Accession No. AAB27103.1 of the GENBANK® biosequence database. Amino acids 8-107 are from CHOP.

SEQ ID NO: 14 is an amino acid sequence of an exemplary CHOP-PEST(3×) design (C-terminally fused to HSVtk) construct. Amino acids 2-101 are from CHOP.

SEQ ID NO: 15 is an amino acid sequence of an exemplary CHOP-PEST(2×) design (C-terminally fused to HSVtk) construct. Amino acids 8-59 are from CHOP.

SEQ ID NO: 16 is an amino acid sequence of an exemplary p21 (CDKN1A) polypeptide. It corresponds to Accession No. NP_000380.1 of the GENBANK® biosequence database.

SEQ ID NO: 17 is an amino acid sequence of an exemplary p21-PEST design (C-terminally fused to HSVtk) construct. Amino acids 25-41 and 63-80 are from Fra-1 and 42-60 are from p21.

SEQ ID NO: 18 is an amino acid sequence of an exemplary fos-related antigen 1 (FRA1) isoform 1 polypeptide. It corresponds to Accession No. NP_005429.1 of the GENBANK® biosequence database. Amino acids 180-196 and 251-270 are from Fra-1.

SEQ ID NO: 19 is an amino acid sequence of an exemplary MEF2A polypeptide. It corresponds to Accession No. AAH53871.1 of the GENBANK® biosequence database.

SEQ ID NO: 20 is an amino acid sequence of an exemplary TLS-CHOP polypeptide. It corresponds to Accession No. AAB27103.1 of the GENBANK® biosequence database.

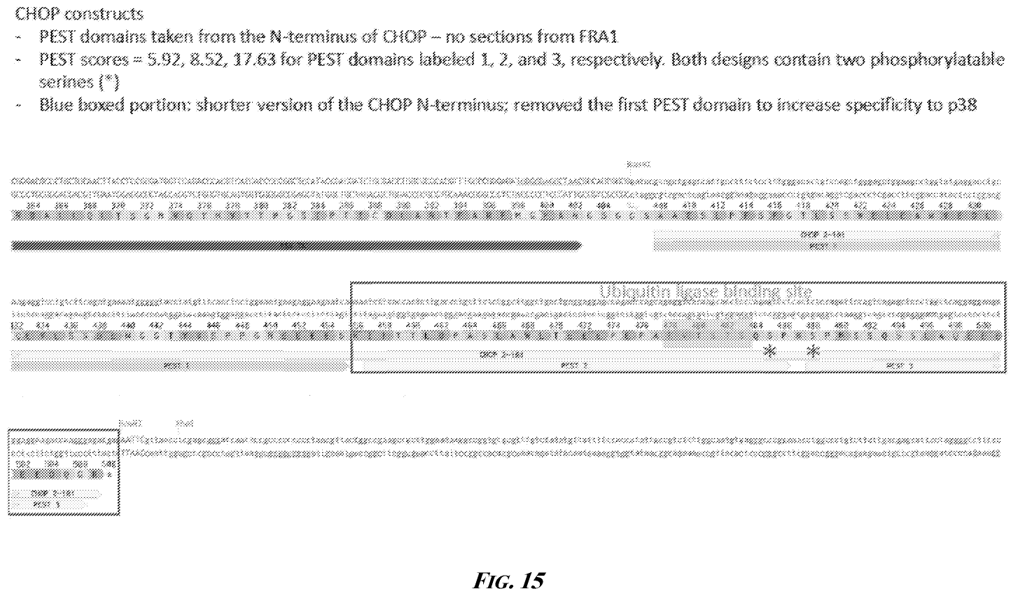

SEQ ID NO: 21 is the nucleotide sequence of the exemplary CHOP construct depicted in FIG. 15.

SEQ ID NO: 22 is the amino acid sequence encoded by SEQ ID NO: 22 and as depicted in FIG. 15.

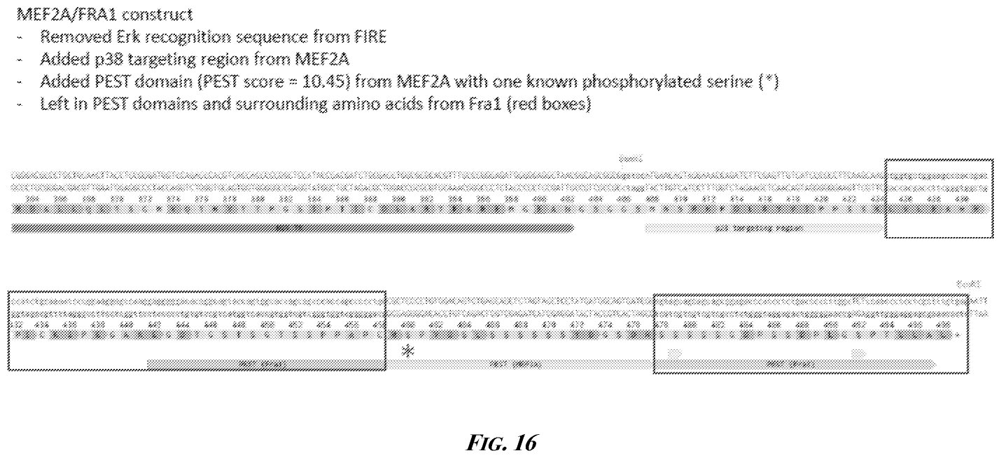

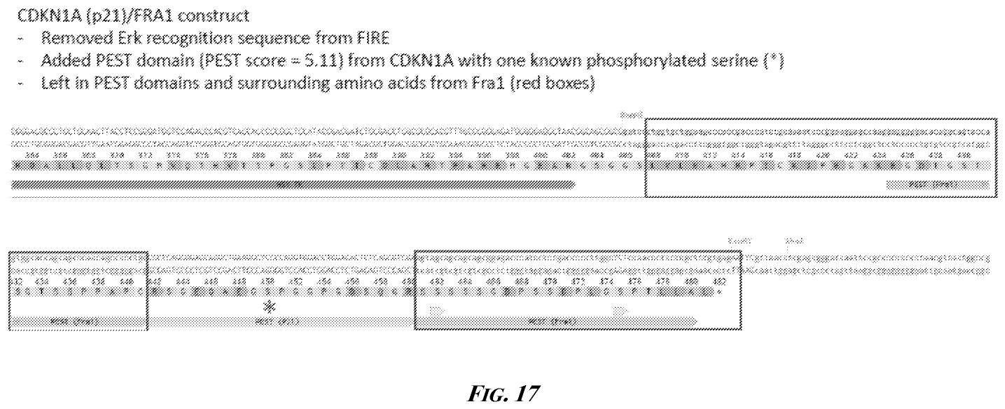

SEQ ID NO: 23 is the nucleotide sequence of the exemplary MEF2A/FRA1 construct depicted in FIG. 16.

SEQ ID NO: 24 is the amino acid sequence encoded by SEQ ID NO: 24 and as depicted in FIG. 16.

SEQ ID NO: 25 is the nucleotide sequence of the exemplary CDKN1A (p21)/FRA1 construct depicted in FIG. 17.

SEQ ID NO: 26 is the amino acid sequence encoded by SEQ ID NO: 26 and as depicted in FIG. 17.

SEQ ID NO: 27 is the nucleotide sequence of the full length exemplary CHOP construct pMSCV-FLAG-NLS-HSVtk-CHOP-IRES-GFP-II.

SEQ ID NO: 28 is the amino acid sequence of the full length exemplary CHOP construct pMSCV-FLAG-NLS-HSVtk-CHOP-IRES-GFP-II.

SEQ ID NO: 29 is the nucleotide sequence of the full length exemplary chop construct pMSCV-FLAG-NLS-HSVtk-chop-IRES-GFP-II.

SEQ ID NO: 30 is the amino acid sequence of the full length exemplary chop construct pMSCV-FLAG-NLS-HSVtk-chop-IRES-GFP-II.

SEQ ID NO: 31 is the nucleotide sequence of the full length exemplary CHOP construct pMSCV-FLAG-NLS-HSVtk-MEF2-IRES-GFP-II.

SEQ ID NO: 32 is the amino acid sequence of the full length exemplary MEF2 construct pMSCV-FLAG-NLS-HSVtk-MEF2-IRES-GFP-II.

SEQ ID NO: 33 is the nucleotide sequence of the full length exemplary CHOP construct pMSCV-FLAG-NLS-HSVtk-p21-IRES-GFP-II.

SEQ ID NO: 34 is the amino acid sequence of the full length exemplary MEF2 construct pMSCV-FLAG-NLS-HSVtk-p21-IRES-GFP-II.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

Headings are included herein for reference and to aid in locating certain sections. These headings are not intended to limit the scope of the concepts described therein under, and these concepts may have applicability in other sections throughout the entire specification.

| | Abbreviations and Acronyms |
|---|---|
| CED: | convection-enhanced delivery |
| EGFR: | epidermal growth factor receptor |
| ERK: | extracellular regulated kinase |
| EV: | empty vector |
| FIRE: | Fra-1 based Integrative Reporter |
| FUS: | focused ultrasound |
| GBM: | glioblastoma multiforme |
| GCV: | ganciclovir |
| GIC: | glioblastoma-initiating cell |
| HSVtk: | Herpes simplex virus thymidine kinase |
| HSVtk-FIRE: | HSVtk-Fra1-based integrative reporter |
| kg: | kilogram |
| mg: | milligram |
| NLS: | nuclear localization sequence |
| pfu: | plaque forming unit(s) |
| RTK: | receptor tyrosine kinase |
| p38 or p38 MAP: | p38 mitogen-activated protein kinase |

### I. DEFINITIONS

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed subject matter and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value or to an amount of a composition, dose, sequence identity (e. g., when comparing two or more nucleotide or amino acid sequences), mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “about”, as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. Therefore, about 50% means in the range of 45%-55%. Numerical ranges recited herein by endpoints include all numbers and fractions subsumed within that range (e. g. 1 to 5 includes 1, 1.5, 2, 2.75, 3, 3.90, 4, and 5). It is also to be understood that all numbers and fractions thereof are presumed to be modified by the term “about”.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The term “gene” refers broadly to any segment of DNA associated with a biological function. A gene can comprise sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

As is understood in the art, a gene comprises a coding strand and a non-coding strand. As used herein, the terms “coding strand”, “coding sequence” and “sense strand” are used interchangeably, and refer to a nucleic acid sequence that has the same sequence of nucleotides as an mRNA from which the gene product is translated. As is also understood in the art, when the coding strand and/or sense strand is used to refer to a DNA molecule, the coding/sense strand includes thymidine residues instead of the uridine residues found in the corresponding mRNA. Additionally, when used to refer to a DNA molecule, the coding/sense strand can also include additional elements not found in the mRNA including, but not limited to promoters, enhancers, and introns. Similarly, the terms “template strand” and “antisense strand” are used interchangeably and refer to a nucleic acid sequence that is complementary to the coding/sense strand.

Similarly, all genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Also encompassed are any and all nucleic acid sequences that encode the disclosed amino acid sequences, including but not limited to those disclosed in the corresponding entries and Accession Nos. of the GENBANK® biosequence database.

The term “gene expression” generally refers to the cellular processes by which a biologically active polypeptide is produced from a DNA sequence and exhibits a biological activity in a cell. As such, gene expression involves the processes of transcription and translation, but also involves post-transcriptional and post-translational processes that can influence a biological activity of a gene or gene product. These processes include, but are not limited to RNA syntheses, processing, and transport, as well as polypeptide synthesis, transport, and post-translational modification of polypeptides. Additionally, processes that affect protein-protein interactions within the cell can also affect gene expression as defined herein.

As used herein, the term “CHOP” refers to the C/EBP Homologous Protein genetic locus and its gene products (e. g., CHOP nucleic acids and polypeptides). It is a transcription factor and p38 substrate. CHOP is activated in cells as part of the endoplasmic reticulum stress-apoptosis pathway. It is phosphorylated by p38 on S79 and S82, both of which are included in the presently disclosed subject matter. Provided in accordance with the presently disclosed subject matter are representative CHOP amino acid and nucleic acid sequences, including but not limited to those as set forth in FIGS. 15-20, a substantially homologous amino acid sequence or substantially homologous nucleic acid sequence thereto, a fragment thereof, or a substantially homologous amino acid sequence or a substantially homologous nucleic acid sequence to a fragment thereof. Also provided are complementary strands of any of these nucleic acid sequences and a nucleic acid sequence differing from any of these nucleic acids due to degeneracy of the genetic code, and which encodes an amino acid sequence encoded by the isolated nucleic acid molecule. Any CHOP amino acid sequence or CHOP nucleic acid sequence as would be apparent to one of ordinary skill in the art upon a review of the instant disclosure is provided in accordance with the presently disclosed subject matter.

As used herein, the term “FRA1” refers to the Fos-related antigen 1 genetic locus and its gene products (e. g., FRA1 nucleic acids and polypeptides). FRA1 is a transcription factor and ERK substrate, and is part of the transcription factor complex AP1. It is phosphorylated by ERK. Provided in accordance with the presently disclosed subject matter are representative FRA1 amino acid and nucleic acid sequences, including but not limited to those as set forth in FIGS. 15-20, a substantially homologous amino acid sequence or substantially homologous nucleic acid sequence thereto, a fragment thereof, or a substantially homologous amino acid sequence or substantially homologous nucleic acid sequence to a fragment thereof. Also provided are complementary strands of any of these nucleic acid sequences and a nucleic acid sequence differing from any of these nucleic acids due to degeneracy of the genetic code, and which encodes an amino acid sequence encoded by the isolated nucleic acid molecule. Any FRA1 amino acid sequence or FRA1 nucleic acid sequence as would be apparent to one of ordinary skill in the art upon a review of the instant disclosure is provided in accordance with the presently disclosed subject matter.

As used herein the term “MEF2A” refers to the myocyte enhancer factor-2A genetic locus and its gene products (e. g., MEF2A nucleic acids and polypeptides). MEF2A is a transcription factor and p38 substrate that is a multi-purpose transcription factor that responds to numerous stress and growth-factor related signals. It is phosphorylated by p38 at T312, T319 and S453 by p38, the last of which is included in the presently disclosed subject matter. Provided in accordance with the presently disclosed subject matter are representative MEF2A amino acid and nucleic acid sequences, including but not limited to those as set forth in FIGS. 15-20, a substantially homologous amino acid sequence or substantially homologous nucleic acid sequence thereto, a fragment thereof, or a substantially homologous amino acid sequence or a substantially homologous nucleic acid sequence to a fragment thereof. Also provided are complementary strands of any of these nucleic acid sequences and a nucleic acid sequence differing from any of these nucleic acids due to degeneracy of the genetic code, and which encodes an amino acid sequence encoded by the isolated nucleic acid molecule. Any MEF2A amino acid sequence or MEF2A nucleic acid sequence as would be apparent to one of ordinary skill in the art upon a review of the instant disclosure is provided in accordance with the presently disclosed subject matter.

As used herein, the term “p21” refers to the p21 genetic locus and its gene products (e. g., p21 nucleic acids and polypeptides). It is also referred to as cyclin-dependent kinase inhibitor 1 (CDKN1A). p21 is a transcription factor and potent regulator of mitosis, and is also a p38 substrate. It is involved in cell cycle arrest in response to DNA damage. p21 can be phosphorylated by p38 or JNK at S130, which is included in the presently disclosed subject matter. Provided in accordance with the presently disclosed subject matter are representative p21 amino acid and nucleic acid sequences, including but not limited to those as set forth in FIGS. 15-20, a substantially homologous amino acid sequence or substantially homologous nucleic acid sequence thereto, a fragment thereof, or a substantially homologous amino acid sequence or substantially homologous nucleic acid sequence to a fragment thereof. Also provided are complementary strands of any of these nucleic acid sequences and a nucleic acid sequence differing from any of these nucleic acids due to degeneracy of the genetic code, and which encodes an amino acid sequence encoded by the isolated nucleic acid molecule. Any p21 amino acid sequence or p21 nucleic acid sequence as would be apparent to one of ordinary skill in the art upon a review of the instant disclosure is provided in accordance with the presently disclosed subject matter.

The terms “modulate” or “alter” are used interchangeably and refer to a change in the expression level of a gene, or a level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator. For example, the terms “modulate” and/or “alter” can mean “inhibit” or “suppress”, but the use of the words “modulate” and/or “alter” are not limited to this definition.

The term “RNA” refers to a molecule comprising at least one ribonucleotide residue. By “ribonucleotide” is meant a nucleotide with a hydroxyl group at the 2′ position of a D-ribofuranose moiety. The terms encompass double stranded RNA, single stranded RNA, RNAs with both double stranded and single stranded regions, isolated RNA such as partially purified RNA, essentially pure RNA, synthetic RNA, recombinantly produced RNA, as well as altered RNA, or analog RNA, that differs from naturally occurring RNA by the addition, deletion, substitution, and/or alteration of one or more nucleotides. Such alterations can include addition of non-nucleotide material, for example at one or more nucleotides of the RNA. Nucleotides in the RNA molecules of the presently disclosed subject matter can also comprise non-standard nucleotides, such as non-naturally occurring nucleotides or chemically synthesized nucleotides or deoxynucleotides. These altered RNAs can be referred to as analogs or analogs of a naturally occurring RNA.

The term “transcription factor” generally refers to a protein that modulates gene expression, such as by interaction with the cis-regulatory element and/or cellular components for transcription, including RNA Polymerase, Transcription Associated Factors (TAFs), chromatin-remodeling proteins, reverse tet-responsive transcriptional activator, and any other relevant protein that impacts gene transcription.

The term “promoter” defines a region within a gene that is positioned 5′ to a coding region of a same gene and functions to direct transcription of the coding region. The promoter region includes a transcriptional start site and at least one cis-regulatory element. The term “promoter” also includes functional portions of a promoter region, wherein the functional portion is sufficient for gene transcription. To determine nucleotide sequences that are functional, the expression of a reporter gene is assayed when variably placed under the direction of a promoter region fragment.

The terms “active”, “functional” and “physiological”, as used for example in “enzymatically active”, “functional” and “physiologically accurate”, and variations thereof, refer to the states of genes, regulatory components, etc. that are reflective of the dynamic states of each as they exist naturally, or in vivo, in contrast to static or non-active states of each. Measurements, detections, or screenings based on the active, functional and/or physiologically relevant states of biological indicators can be useful in elucidating a mechanism, or defining a disease state or phenotype, as it occurs naturally. This is in contrast to measurements taken based on static concentrations or quantities of a biological indicator that are not reflective of level of activity or function thereof.

The term “substantially identical”, as used herein to describe a degree of similarity between nucleotide sequences, peptide sequences and/or amino acid sequences refers to two or more sequences that have in some embodiments at least about least 60%, in some embodiments at least about 70%, in some embodiments at least about 80%, in some embodiments at least about 85%, in some embodiments at least about 90%, in some embodiments at least about 91%, in some embodiments at least about 92%, in some embodiments at least about 93%, in some embodiments at least about 94%, in some embodiments at least about 95%, in some embodiments at least about 96%, in some embodiments at least about 97%, in some embodiments at least about 98%, in some embodiments at least about 99%, in some embodiments about 90% to about 99%, and in some embodiments about 95% to about 99% nucleotide identity, when compared and aligned for maximum correspondence, as measured using a sequence comparison algorithm or by visual inspection.

The terms “additional therapeutically active compound” or “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As use herein, the terms “administration of” and or “administering” a compound should be understood to mean providing a composition of the presently disclosed subject matter or a prodrug of the presently disclosed subject matter to a subject in need of treatment.

The term “adult” as used herein, is meant to refer to any non-embryonic or non-juvenile subject.

Cells or tissue are “affected” by an injury, disease, or disorder if the cells or tissue have an altered phenotype relative to the same cells or tissue in a subject not afflicted with the injury, disease, condition, or disorder.

As used herein, an “agonist” is a composition of matter that, when administered to a mammal such as a human, enhances or extends a biological activity of interest. Such effect may be direct or indirect.

A disease, condition, or disorder is “alleviated” if the severity of a symptom of the disease or disorder, the frequency with which such a symptom is experienced by a patient, or both, are reduced.

As used herein, “alleviating an injury, disease or disorder symptom”, means reducing the frequency or severity of the symptom.

As used herein, amino acids are represented by the full name thereof, by the three letter code corresponding thereto, or by the one-letter code corresponding thereto, as indicated in the following table:

| | 3-Letter | 1-Letter | |
|---|---|---|---|
| Full Name | Code | Code | Functionally Equivalent Codons |
| Aspartic Acid | Asp | D | GAC GAU |
| Glutamic Acid | Glu | E | GAA GAG |
| Lysine | Lys | K | AAA AAG |
| Arginine | Arg | R | AGA AGG CGA CGC CGG CGU |
| Histidine | His | H | CAC CAU |
| Tyrosine | Tyr | Y | UAC UAU |
| Cysteine | Cys | C | UGC UGU |
| Asparagine | Asn | N | AAC AAU |
| Glutamine | Gln | Q | CAA CAG |
| Serine | Ser | S | ACG AGU UCA UCC UCG UCU |
| Threonine | Thr | T | ACA ACC ACG ACU |
| Glycine | Gly | G | GGA GGC GGG GGU |
| Alanine | Ala | A | GCA GCC GCG GCU |
| Valine | Val | V | GUA GUC GUG GUU |
| Leucine | Leu | L | UUA UUG CUA CUC CUG CUU |
| Isoleucine | Ile | I | AUA AUC AUU |
| Methionine | Met | M | AUG |
| Proline | Pro | P | CCA CCC CCG CCU |
| Phenylalanine | Phe | F | UUC UUU |
| Tryptophan | Trp | W | UGG |

The term “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

The term “amino acid” is used interchangeably with “amino acid residue”, and may refer to a free amino acid and to an amino acid residue of a peptide. It will be apparent from the context in which the term is used whether it refers to a free amino acid or a residue of a peptide.

Amino acids have the following general structure:

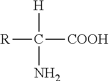

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e. g., 5-fluorouracil is an analog of thymine).

An “antagonist” is a composition of matter that when administered to a mammal such as a human, inhibits or impedes a biological activity attributable to the level or presence of an endogenous compound in the mammal. Such effect may be direct or indirect.

The term “antibody”, as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms including, for example, polyclonal antibodies, monoclonal antibodies, Fv, Fab and F(ab)₂, as well as single chain antibodies and humanized antibodies.

The term “antibody”, as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms including, for example, polyclonal antibodies, monoclonal antibodies, Fv, Fab and F(ab)₂, as well as single chain antibodies and humanized antibodies (Harlow & Lane, 1999; Harlow et al., 1989; Houston et al., 1988; Bird et al., 1988.

An “antibody heavy chain”, as used herein, refers to the larger of the two types of polypeptide chains present in all antibody molecules.

An “antibody light chain”, as used herein, refers to the smaller of the two types of polypeptide chains present in all antibody molecules.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically competent cells, or both. An antigen can be derived from organisms, subunits of proteins/antigens, killed or inactivated whole cells or lysates.

A ligand or a receptor (e. g., an antibody) “specifically binds to” or “is specifically immunoreactive with” a compound when the ligand or receptor functions in a binding reaction which is determinative of the presence of the compound in a sample of heterogeneous compounds. Thus, under designated assay (e. g., immunoassay) conditions, the ligand or receptor binds preferentially to a particular compound and does not bind in a significant amount to other compounds present in the sample. For example, a polynucleotide specifically binds under hybridization conditions to a compound polynucleotide comprising a complementary sequence; an antibody specifically binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow & Lane, 1988 for a description of immunoassay formats and conditions that can be used to determine specific immunoreactivity.

The term “antimicrobial agents” as used herein refers to any naturally occurring, synthetic, or semi-synthetic compound or composition or mixture thereof, which is safe for human or animal use as practiced in the methods of the presently disclosed subject matter, and is effective in killing or substantially inhibiting the growth of microbes. “Antimicrobial” as used herein, includes antibacterial, antifungal, and antiviral agents.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. Antisense oligonucleotides include, but are not limited to, phosphorothioate oligonucleotides and other modifications of oligonucleotides.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the term “biologically active fragments” or “bioactive fragment” of the polypeptides encompasses natural or synthetic portions of the full-length protein that are capable of specific binding to their natural ligand or of performing the function of the protein.

The term “biological sample”, as used herein, refers to samples obtained from a living organism, including skin, hair, tissue, blood, plasma, cells, sweat, and urine.

A “biomarker” is a specific biochemical in the body which has a particular molecular feature that makes it useful for measuring the progress of disease or the effects of treatment, or for measuring a process of interest.

As used herein, the term “carrier molecule” refers to any molecule that is chemically conjugated to the antigen of interest that enables an immune response resulting in antibodies specific to the native antigen.

As used herein, the term “chemically conjugated”, or “conjugating chemically” refers to linking one chemical entity to another entity, e. g. two polypeptide sequences. This linking can occur on the genetic level using recombinant technology, wherein a hybrid protein may be produced containing the amino acid sequences, or portions thereof, of one or both of the polypeptide sequences. This hybrid protein is produced by an oligonucleotide sequence encoding both the two polypeptide sequences, and/or portions thereof. This linking also includes covalent bonds created between the chemical entities, e. g. two polypeptide sequences, using other chemical reactions, such as, but not limited to glutaraldehyde reactions. Covalent bonds may also be created using a third molecule bridging the two polypeptide sequences. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the two chemical entities, e. g., two polypeptide sequences.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. As used herein, the terms “complementary” or “complementarity” are used in reference to polynucleotides (i. e., a sequence of nucleotides) related by the base-pairing rules. For example, the sequence 5′-AGT-3′ is complementary to the sequence 5′-ACT-3′.

Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and in some embodiments at least about 75%, at least about 90%, or at least about 95% of the nucleotide residues of the first portion are capable of base pairing with nucleotide residues in the second portion. In some embodiments, all nucleotide residues of the first portion are capable of base pairing with nucleotide residues in the second portion.

The term “complex”, as used herein in reference to proteins, refers to binding or interaction of two or more proteins. Complex formation or interaction can include such things as binding, changes in tertiary structure, and modification of one protein by another, such as phosphorylation.

A “compound”, as used herein, refers to any type of substance or agent that is commonly considered a drug, or a candidate for use as a drug, as well as combinations and mixtures of the above.

As used herein, the term “conservative amino acid substitution” is defined herein as an amino acid exchange within one of the following five groups:
I. Small aliphatic, nonpolar, or slightly polar residues:
   Ala, Ser, Thr, Pro, Gly;
II. Polar, negatively charged residues and their amides:
   Asp, Asn, Glu, Gln;
III. Polar, positively charged residues:
   His, Arg, Lys;
IV. Large, aliphatic, nonpolar residues:
   Met Leu, Ile, Val, Cys
V. Large, aromatic residues:
   Phe, Tyr, Trp

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell, tissue, sample, or subject is one being examined or treated.

“Cytokine”, as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, chemokines, protein or peptide hormones, and transforming growth factors. A number of other cytokines are known to those of skill in the art. The sources, characteristics, targets, and effector activities of these cytokines have been described.

The term “delivery vehicle” refers to any kind of device or material which can be used to deliver compounds in vivo or can be added to a composition comprising compounds administered to a plant or animal. This includes, but is not limited to, implantable devices, aggregates of cells, matrix materials, gels, nucleic acids, etc.

As used herein, a “derivative” of a compound, when referring to a chemical compound, is one that may be produced from another compound of similar structure in one or more steps, as in replacement of H by an alkyl, acyl, or amino group.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e. g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, the term “diagnosis” refers to detecting a disease or disorder or a risk or propensity for development of a disease or disorder, for the types of diseases or disorders encompassed by the presently disclosed subject matter. In any method of diagnosis there exist false positives and false negatives. Any one method of diagnosis does not provide 100% accuracy.

A “disease” is a state of health of an animal wherein the subject cannot maintain homeostasis, and wherein if the disease is not ameliorated then the subject's health continues to deteriorate. In contrast, a “disorder” in a subject is a state of health in which the animal is able to maintain homeostasis, but in which the subject's state of health is less favorable than it would be in the absence of the disorder. Left untreated, a disorder does not necessarily cause a further decrease in the subject's state of health.

As used herein, the term “domain” refers to a part of a molecule or structure that shares common physicochemical features, such as, but not limited to, hydrophobic, polar, globular, and helical domains or properties such as ligand binding, signal transduction, cell penetration and the like. Specific examples of binding domains include, but are not limited to, DNA binding domains and ATP binding domains. As used herein, the term “effector domain” refers to a domain capable of directly interacting with an effector molecule, chemical, or structure in the cytoplasm which is capable of regulating a biochemical pathway.

The term “downstream” when used in reference to a direction along a nucleotide sequence means the 5′ to 3′ direction. Similarly, the term “upstream” means the 3′ to 5′ direction.

As used herein, an “effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i. e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “enhancer” is a DNA regulatory element that can increase the efficiency of transcription, regardless of the distance or orientation of the enhancer relative to the start site of transcription.

As used herein, an “essentially pure” preparation of a particular protein or peptide is a preparation wherein in some embodiments at least about 95%, and in some embodiments at least about 99%, by weight, of the protein or peptide in the preparation is the particular protein or peptide.

As used in the specification and the appended claims, the terms “for example”, “for instance”, “such as”, “including” and the like are meant to introduce examples that further clarify more general subject matter. Unless otherwise specified, these examples are provided only as an aid for understanding the presently disclosed subject matter and are not meant to be limiting in any fashion.

The terms “formula” and “structure” are used interchangeably herein.

As used herein the term “expression” when used in reference to a gene or protein, without further modification, is intended to encompass transcription of a gene and/or translation of the transcript into a protein.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment”, as applied to a protein or peptide, can ordinarily be at least about 2-15 amino acids in length, at least about 15-25 amino acids, at least about 25-50 amino acids in length, at least about 50-75 amino acids in length, at least about 75-100 amino acids in length, and greater than 100 amino acids in length, depending on the particular protein or peptide being referred to.

As used herein, the term “fragment” as applied to a nucleic acid, may ordinarily be in some embodiments at least about 20 nucleotides in length, in some embodiments at least about 50 nucleotides, in some embodiments from about 50 to about 100 nucleotides, in some embodiments at least about 100 to about 200 nucleotides, in some embodiments at least about 200 nucleotides to about 300 nucleotides, in some embodiments at least about 300 to about 350, in some embodiments at least about 350 nucleotides to about 500 nucleotides, in some embodiments at least about 500 to about 600, in some embodiments at least about 600 nucleotides to about 620 nucleotides, in some embodiments at least about 620 to about 650, and in some embodiments, the nucleic acid fragment will be greater than about 650 nucleotides in length.

As used herein, a “functional” molecule is a molecule in a form in which it exhibits a property or activity by which it is characterized. A functional enzyme, for example, is one that exhibits the characteristic catalytic activity by which the enzyme is characterized.

“Homologous” or “homolog”, as used herein, refers to the subunit sequence similarity between two polymeric molecules, e. g., between two nucleic acid molecules, e. g., two DNA molecules or two RNA molecules, or between two polypeptide molecules. When a subunit position in both of the two molecules is occupied by the same monomeric subunit, e. g., if a position in each of two DNA molecules is occupied by adenine, then they are homologous at that position. The homology between two sequences is a direct function of the number of matching or homologous positions, e. g., if half (e. g., five positions in a polymer ten subunits in length) of the positions in two compound sequences are homologous then the two sequences are 50% homologous, if 90% of the positions, e. g., 9 of 10, are matched or homologous, the two sequences share 90% homology. By way of example, the DNA sequences 3′ATTGCC5′ and 3′TATGGC share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990, as modified in Karlin & Altschul, 1993. This algorithm is incorporated into the NBLAST and XBLAST programs of Altschul et al., 1990a, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al; 1997. Alternatively, PSI-Blast or PHI-Blast can be used to perform an iterated search which detects distant relationships between molecules (Altschul et al., 1997) and relationships between molecules which share a common pattern. When utilizing BLAST, Gapped BLAST, PSI-Blast, and PHI-Blast programs, the default parameters of the respective programs (e. g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i. e., the strength of the association between the nucleic acids) is impacted by such factors as the degree of complementarity between the nucleic acids, stringency of the conditions involved, the length of the formed hybrid, and the G:C ratio within the nucleic acids.

The term “inhibit”, as used herein, refers to the ability of a compound, agent, or method to reduce or impede a described function, level, activity, rate, etc., based on the context in which the term “inhibit” is used. In some embodiments, inhibition is by at least 10%, in some embodiments by at least 25%, in some embodiments by at least 50%, and in some embodiments, the function is inhibited by at least 75%. The term “inhibit” is used interchangeably with “reduce” and “block”.

The term “inhibit a protein”, as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting or applying” includes administration of a composition of the presently disclosed subject matter by any number of routes and means including, but not limited to, topical, oral, buccal, intravenous, intramuscular, intra-arterial, intramedullary, intracranial, intratumoral, intrathecal, intraventricular, transdermal, subcutaneous, intraperitoneal, intranasal, enteral, topical, sublingual, vaginal, ophthalmic, pulmonary, or rectal means.

The term “injected once with a 5-daily dose”, as used herein, means that an induction therapy was initiated wherein mice were injected with 1 g protein once a day for five consecutive days and then followed over time as indicated.

As used herein, an “instructional material” includes a publication, a recording, a diagram, or any other medium of expression which can be used to communicate the usefulness of the peptide of the presently disclosed subject matter in the kit for effecting alleviation of the various diseases or disorders recited herein. Optionally, or alternately, the instructional material may describe one or more methods of alleviating the diseases or disorders in a cell or a tissue of a mammal. The instructional material of the kit of the presently disclosed subject matter may, for example, be affixed to a container which contains the identified compound presently disclosed subject matter or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e. g., a DNA fragment which has been removed from the sequences which are normally adjacent to the fragment, e. g., the sequences adjacent to the fragment in a genome in which it naturally occurs. The term also applies to nucleic acids which have been substantially purified from other components which naturally accompany the nucleic acid, e. g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e. g., as a cDNA or a genomic or cDNA fragment produced by PCR or restriction enzyme digestion) independent of other sequences. It also includes a recombinant DNA which is part of a hybrid gene encoding additional polypeptide sequence.

A “ligand” is a compound that specifically binds to a target receptor.

A “receptor” is a compound that specifically binds to a ligand.

A ligand or a receptor “specifically binds to” or “is specifically immunoreactive with” a compound when the ligand or receptor functions in a binding reaction which is determinative of the presence of the compound in a sample of heterogeneous compounds. Thus, under designated assay conditions, the ligand or receptor binds preferentially to a particular compound and does not bind in a significant amount to other compounds present in the sample. For example, a polynucleotide specifically binds under hybridization conditions to a compound polynucleotide comprising a complementary sequence; an antibody specifically binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow & Lane, 1988 for a description of immunoassay formats and conditions that can be used to determine specific immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e. g., through ionic or hydrogen bonds or van der Waals interactions.

“Malexpression” of a gene means expression of a gene in a cell of a patient afflicted with a disease or disorder, wherein the level of expression (including non-expression), the portion of the gene expressed, or the timing of the expression of the gene with regard to the cell cycle, differs from expression of the same gene in a cell of a patient not afflicted with the disease or disorder. It is understood that malexpression may cause or contribute to the disease or disorder, be a symptom of the disease or disorder, or both.

The term “material” refers to any compound, molecule, substance, or group or combination thereof that forms any type of structure or group of structures during or after electroprocessing. Materials include natural materials, synthetic materials, or combinations thereof. Naturally occurring organic materials include any substances naturally found in the body of plants or other organisms, regardless of whether those materials have or can be produced or altered synthetically. Synthetic materials include any materials prepared through any method of artificial synthesis, processing, or manufacture. In some embodiments, the materials are biologically compatible materials.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc., and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “modulate”, as used herein, refers to changing the level of an activity, function, or process. The term “modulate” encompasses both inhibiting and stimulating an activity, function, or process.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, the term “nucleic acid” encompasses RNA as well as single and double-stranded DNA and cDNA. Furthermore, the terms, “nucleic acid”, “DNA”, “RNA” and similar terms also include nucleic acid analogs, i. e. analogs having other than a phosphodiester backbone. For example, the so-called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally no greater than about 50 nucleotides. It will be understood that when a nucleotide sequence is represented by a DNA sequence (i. e., A, T, G, C), this also includes an RNA sequence (i. e., A, U, G, C) in which “U” replaces “T”.

“Operably linked” refers to a juxtaposition wherein the components are configured so as to perform their usual function. Thus, control sequences or promoters operably linked to a coding sequence are capable of effecting the expression of the coding sequence. By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a cell and/or of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a cell and/or-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides.

The term “per application” as used herein refers to administration of a compositions, drug, or compound to a subject.

“Permeation enhancement” and “permeation enhancers” as used herein relate to the process and added materials which bring about an increase in the permeability of skin to a poorly skin permeating pharmacologically active agent, i. e., so as to increase the rate at which the drug permeates through the skin and enters the bloodstream. “Permeation enhancer” is used interchangeably with “penetration enhancer”.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan. As used herein, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i. e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e. g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a cell and/or specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. A “significant detectable level” is an amount of contaminate that would be visible in the presented data and would need to be addressed/explained during analysis of the forensic evidence.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e. g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-β-galactoside to the medium (see Gerhardt et al., 1994, page 574).

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

As used herein, the term “secondary antibody” refers to an antibody that binds to the constant region of another antibody (the primary antibody).

By the term “signal sequence” is meant a polynucleotide sequence which encodes a peptide that directs the path a polypeptide takes within a cell, i. e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By “small interfering RNAs (siRNAs)” is meant, inter alia, an isolated dsRNA molecule comprised of both a sense and an anti-sense strand. In some embodiments, it is greater than 10 nucleotides in length. siRNA also refers to a single transcript which has both the sense and complementary antisense sequences from the target gene, e. g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

As used herein, the term “solid support” relates to a solvent insoluble substrate that is capable of forming linkages (in some embodiments, covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “specifically binds to”, as used herein, is meant when a compound or ligand functions in a binding reaction or assay conditions which is determinative of the presence of the compound in a sample of heterogeneous compounds.

The term “standard”, as used herein, refers to something used for comparison. For example, a standard can be a known standard agent or compound which is administered or added to a control sample and used for comparing results when measuring said compound in a test sample. In some embodiments, the standard compound is added or prepared at an amount or concentration that is equivalent to a normal value for that compound in a normal subject. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, in some embodiments a human.

As used herein, a “subject in need thereof” is a patient, animal, mammal, or human, who will benefit from the method of the presently disclosed subject matter.

As used herein, a “substantially homologous amino acid sequence” refers to homologs where the percentage of identity between the substantially similar amino acid sequence and the reference amino acid sequence is at least about 30%, 40%, 50%, 65%, 75%, 85%, 95%, 96%, 97%, 98%, 99% or more. As used herein, a “substantially homologous amino acid sequence” includes those amino acid sequences which have in some embodiments at least about 95% homology, in some embodiments at least about 96% homology, in some embodiments at least about 97% homology, in some embodiments at least about 98% homology, and in some embodiments at least about 99% homology to an amino acid sequence of a reference sequence. Amino acid sequences similarity or identity can be computed using, for example, the BLASTP and TBLASTN programs which employ the BLAST (basic local alignment search tool) algorithm. The default setting used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e. g., where only changes in amino acids not significantly affecting the peptide function occur. In some embodiments, the substantially similar nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 96%, 97%, 98%, 99% or more. Substantial similarity of nucleic acid sequences can be determined by comparing the sequence identity of two sequences, for example by physical/chemical methods (i. e., hybridization) or by sequence alignment via computer algorithm. Suitable nucleic acid hybridization conditions to determine if a nucleotide sequence is substantially similar to a reference nucleotide sequence are: in some embodiments 7% sodium dodecyl sulfate SDS, 0.5 M NaPO₄, 1 mM EDTA at 50° C. with washing in 2× standard saline citrate (SSC), 0.1% SDS at 50° C.; in some embodiments in 7% (SDS), 0.5 M NaPO₄, 1 mM EDTA at 50° C. with washing in 1×SSC, 0.1% SDS at 50° C.; in some embodiments 7% SDS, 0.5 M NaPO₄, 1 mM EDTA at 50° C. with washing in 0.5×SSC, 0.1% SDS at 50° C.; and in some embodiments in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCG program package (Devereux et al., 1984), and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

The term “substantially pure” describes a compound, e. g., a protein or polypeptide which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e. g., in the case of polypeptides by column chromatography, gel electrophoresis, or HPLC analysis. A compound, e. g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing, eliminating, or preventing those signs.

A “therapeutically effective amount” of a compound is that amount of compound which is sufficient to provide a beneficial effect to the subject to which the compound is administered.

“Tissue” means (1) a group of similar cells united to perform a specific function; (2) a part of an organism consisting of an aggregate of cells having a similar structure and function; or (3) a grouping of cells that are similarly characterized by their structure and function, such as muscle or nerve tissue.

The term “transfection” is used interchangeably with the terms “gene transfer” “, transformation”, and “transduction”, and means the intracellular introduction of a polynucleotide. “Transfection efficiency” refers to the relative amount of the transgene taken up by the cells subjected to transfection. In practice, transfection efficiency is estimated by the amount of the reporter gene product expressed following the transfection procedure.

The term “transgene” is used interchangeably with “inserted gene”, or “expressed gene” and, where appropriate, “gene”. “Transgene” refers to a polynucleotide that, when introduced into a cell, is capable of being transcribed under appropriate conditions so as to confer a beneficial property to the cell such as, for example, expression of a therapeutically useful protein. It is an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, a “transgenic cell” is any cell that comprises a nucleic acid sequence that has been introduced into the cell in a manner that allows expression of a gene encoded by the introduced nucleic acid sequence.

As used herein, the term “transgenic mammal” means a mammal, the germ cells of which comprise an exogenous nucleic acid.

The term to “treat”, as used herein, means reducing the frequency with which symptoms are experienced by a patient or subject or administering an agent or compound to reduce the frequency with which symptoms are experienced.

As used herein, the term “treating” may include prophylaxis of the specific injury, disease, disorder, or condition, or alleviation of the symptoms associated with a specific injury, disease, disorder, or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease and should be interpreted based on the context of the use.

“Treating” is used interchangeably with “treatment” herein.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e. g., naked or contained in liposomes) and viruses that incorporate the recombinant polynucleotide.

One of skill in the art will appreciate that the superiority of the compositions and methods of the presently disclosed subject matter relative to the compositions and methods of the prior art are unrelated to the physiological accuracy of the theory explaining the superior results.

### II. SUBJECTS

The subject treated. screened, tested, or from which a sample is taken, is desirably a human subject, although it is to be understood that the principles of the disclosed subject matter indicate that the compositions and methods are effective with respect to invertebrate and to vertebrate species, including mammals, which are intended to be included in the term “subject”. Moreover, a mammal is understood to include any mammalian species in which screening is desirable, particularly agricultural and domestic mammalian species.

The disclosed methods are particularly useful in the testing, screening and/or treatment of warm-blooded vertebrates. Thus, the presently disclosed subject matter concerns mammals and birds.

More particularly, provided herein is the testing, screening and/or treatment of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i. e., poultry, such as turkeys, chickens, ducks, geese, guinea fowl, and the like, as they are also of economical importance to humans. Thus, provided herein is the treatment of livestock, including, but not limited to, domesticated swine (pigs and hogs), ruminants, horses, poultry, and the like.

In some embodiments, the subject to be used in accordance with the presently disclosed subject matter is a subject in need of treatment and/or diagnosis. In some embodiments, a subject can have or be believed to have a cancer.

### III. EXEMPLARY EMBODIMENTS

The presently disclosed subject matter pertains in some embodiments to methods and compositions for treating cancer in a subject in need thereof comprising administering to the subject a fusion protein construct inserted into an expression vector backbone, such as a viral vector backbone, such as a retroviral expression vector backbone. The vector can be transfected into packaging cell lines to produce viral particles, such as retroviral particles, that can then be injected where target cells are present. In some embodiments, target cells are undergoing a stress response. In some embodiments, the target cells are tumor cells that have been subjected to a treatment, such as but not limited to chemotherapy. In some embodiments, the ERK-dependent tumor cells are present. In some embodiments, they are administered locally. In some embodiments, the vector encodes an HSVtk fused to a nuclear localization sequence and a peptide domain sequence. In some embodiments, amino acids 163-271 of Fra-1 can be fused in-frame to the C-terminus of a therapeutic protein, with a nuclear localization sequence (NLS) at the N-terminus. PCR techniques can be used to amplify therapeutic polypeptide sequences. NLSs, and sequences encoding peptide domains that are stabilized when phosphorylated by by the kinase activity, such as but not limited to extracellular regulated kinase (ERK) activity or p38 MAP kinase activity using the sequences disclosed herein as templates. In some embodiments, a nuclear localization sequence is selected from the group consisting of MAPKKKRK (SEQ ID NO: 7); PKKKRKV (SEQ ID NO: 8); KRPAATKKAGQAKKKK (SEQ ID NO: 9); and PAAKRVKLD (SEQ ID NO: 10).

In FIGS. 15-20, schematics of suicide gene approaches are disclosed. Referring to FIG. 19A, a schematic of ERK-regulated suicide gene activity is presented. ERK is often constitutively active in cancer cells, particularly in glioblastoma cells expressing EGFRvIII. HSVtk is preferentially activated in cells with high ERK. Suicide gene therapy involving herpes simplex virus thymidine kinase (HSVtk) is shown as a representative example.

Referring to FIG. 19B, a schematic of p38-regulated suicide gene activity is presented. p38 is overexpressed in some cancers, including glioblastoma. p38 is activated in response to cell stress, such as chemotherapy-induced DNA damage. In some embodiments, the presently disclosed subject matter pertains to the ability for p38-driven suicide gene therapies to synergize with chemotherapy and the degree of positive feedback driven by HSVtk-driven DNA damage.

In some embodiments of the presently disclosed subject a suicide gene suicide gene stabilized by p38 can be used cooperatively with chemotherapy to kill cancer cells.HSVtk fusion proteins are validated with stability dependent on p38 phosphorylation. Representative criteria for PEST domain design include that the it must be predicted to have a high PEST score (PEST=proline, glutamate, serine, threonine). Further the domain should come from a protein primarily phosphorylated by p38 and have at least one phosphorylatable serine in the sequence.

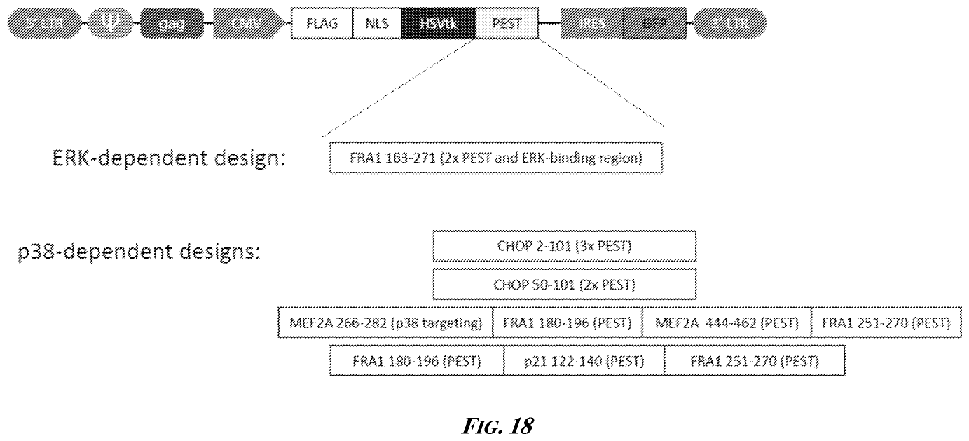

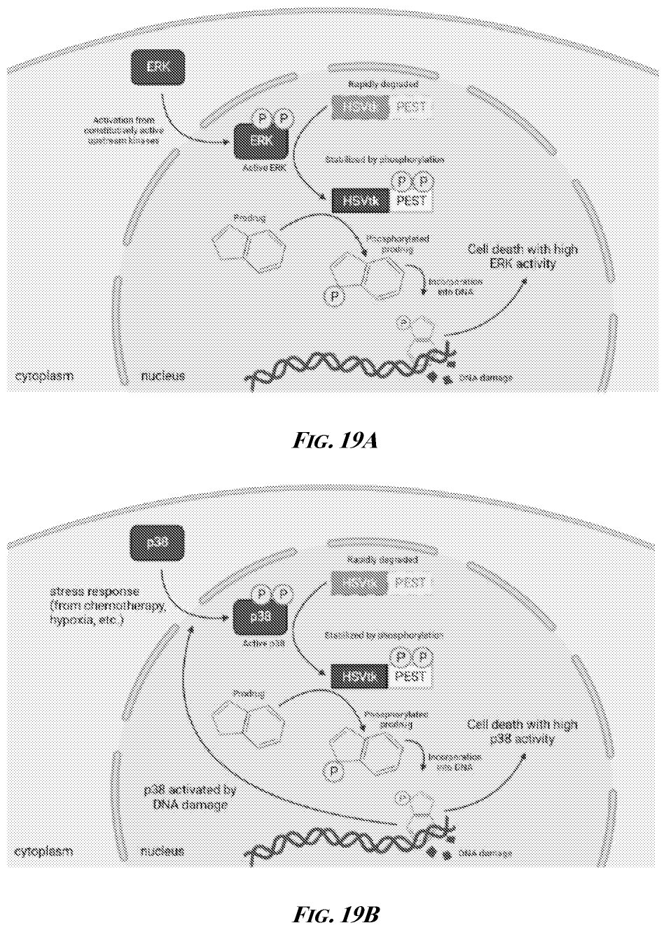

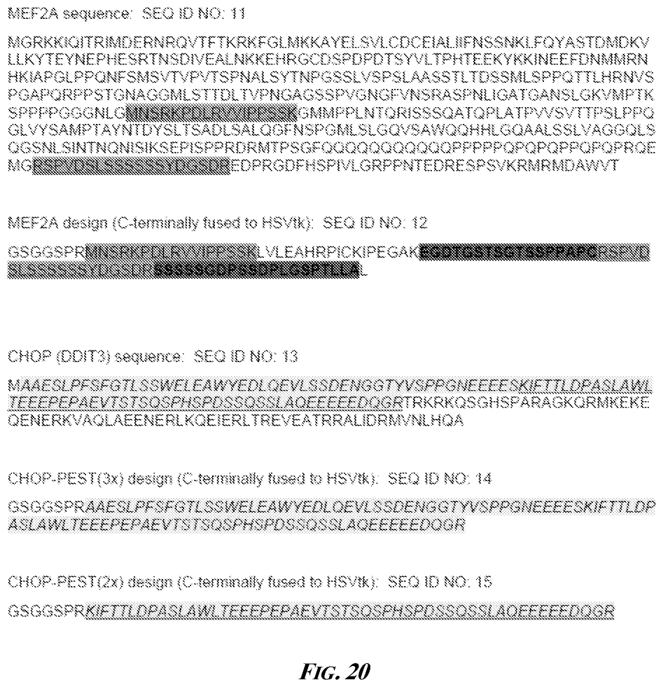

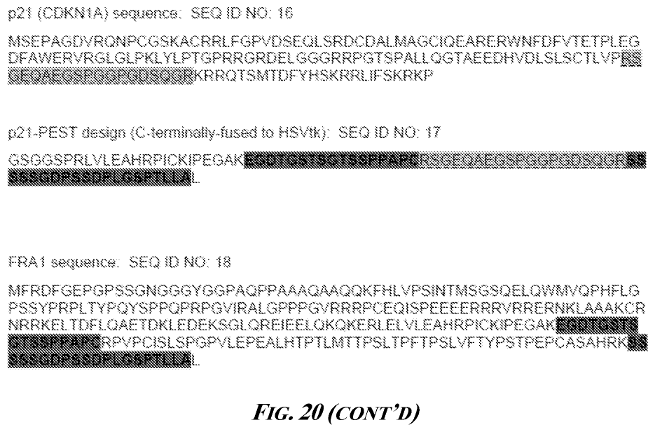

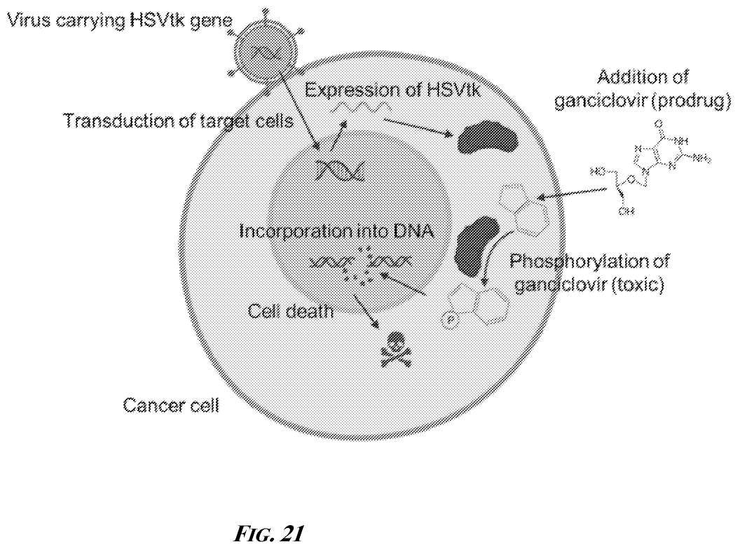

Referring to FIGS. 18 and 20, vector constructs of the presently disclosed subject matter and sequence employed in the vector constructs are described. In some embodiments, the designs for p38-targetable sequences come from MEF2A, CHOP, or p21, which are all proteins with well-characterized p38 phosphorylation sites. The two designs based on the CHOP protein contain no overlap with the FRA1-based design. The MEF2A and p21 designs incorporate PEST domains from FRA1 but have removed the ERK-targeting sequence to prevent ERK binding. They contain PEST domains from their respective proteins with at least one known serine that can be phosphorylated by p38. The MEF2A design also includes a sequence that p38 binds to before phosphorylating MEF2A.

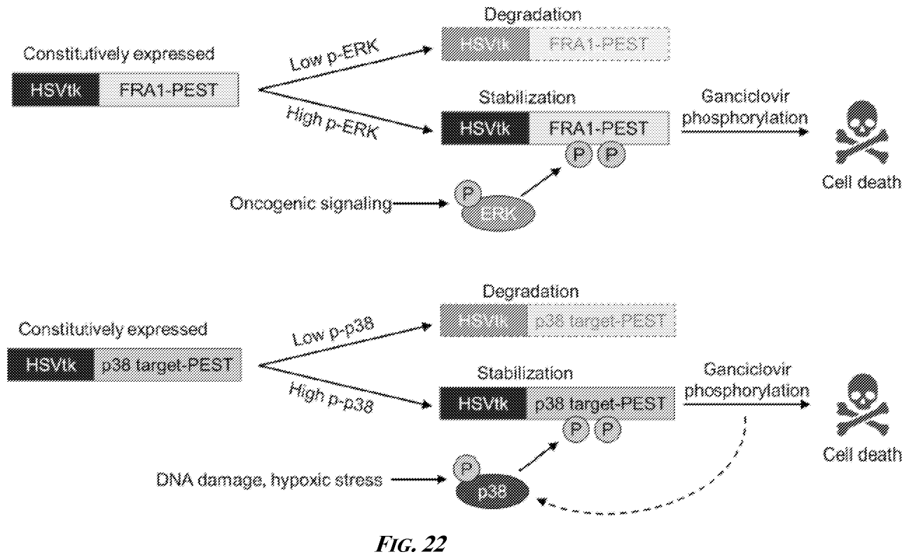

In some embodiments, CHOP constructs were prepared as follows. PEST domains were taken from the N-terminus of CHOP, with no sections from FRA1. PEST scores were 5.92, 8.52, and 17.63, respectively, for PEST domains labeled 1, 2, and 3, respectively. Both designs contain two phosphorylatable serines (show in Figures, including FIGS. 15-17 and 22). In some embodiments, a shorter version of the CHOP N-terminus was employed, removing the PEST domain to increase specificity to p38. The construct also included a Ub ligase binding site (Zhang et al., 2014) and p38 phosphorylation (Wang & Ron, 1996).

In some embodiments, a MEF2A/FRA1 construct was prepared wherein an Erk recognition sequence was removed from FIRE. A p38 targeting region from MEF2A was included, and a PEST domain (PEST score=10.45) from MEF2A with one known phosphorylated serine (*) was included. PEST domains and surrounding amino acids from Fra1 were left in. The construct also includes p38 targeting (Yang et al., 1999) and p38 phosphorylation (Zhao et al., 1999).

In some embodiments, a CDKN1A (p21)/FRA1 construct was prepared, wherein an Erk recognition sequence from FIRE was removed. A PEST domain (PEST score=5.11) from CDKN1A with one known phosphorylated serine (*) was added. PEST domains and surrounding amino acids from Fra1 were left in. The construct also includes p38 phosphorylation (Kim et al., 2002).

Referring to FIG. 20, highlighted sequences are the sequences taken from the protein sequences discussed above. Double underlined sequences in FIG. 20 are the sequences from MEF2A (SEQ ID NO: 11). SEQ ID NO: 12 shows a MEF2A design wherein C-terminally fused to HSVtk. In FIG. 20, Italicized sequences are from CHOP, with the underlined sequence being used in both the full length and shortened CHOP-PEST sequences). SEQ ID NO: 13 shows a CHOP (DDIT3) sequence and SEQ ID NO: 14 shows CHOP-PEST(3×) design (C-terminally fused to HSVtk). SEQ ID NO: 15 shows CHOP-PEST(2×) design (C-terminally fused to HSVtk). In FIG. 19, sequences with dashed underlining are from p21 and sequences in bold are from FRA1. These sequences were also used in the previous ERK-dependent design). SEQ ID NO: 16 shows a p21 (CDKN1A) sequence. SEQ ID NO: 18 shows a FRA1 sequence.

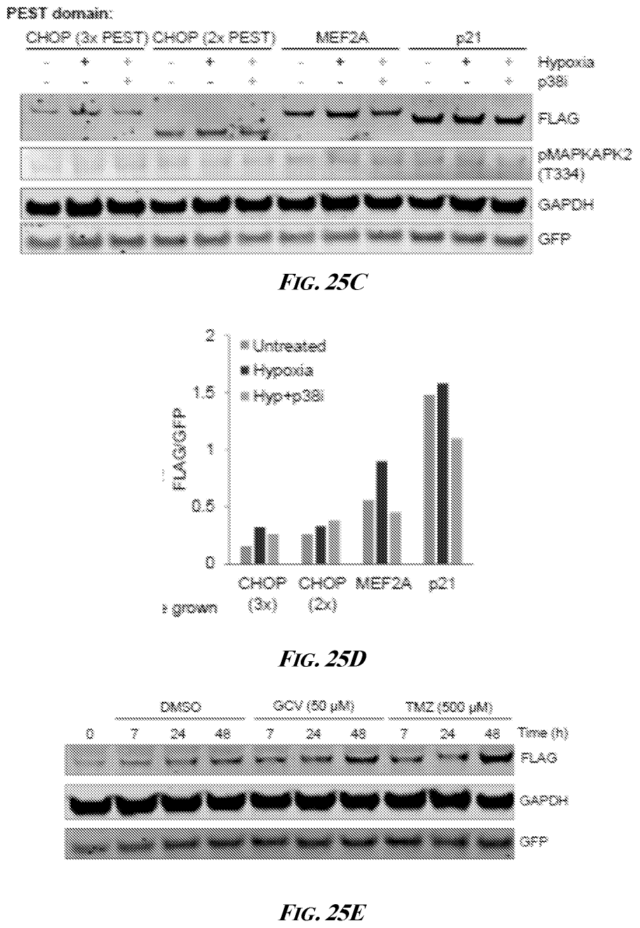

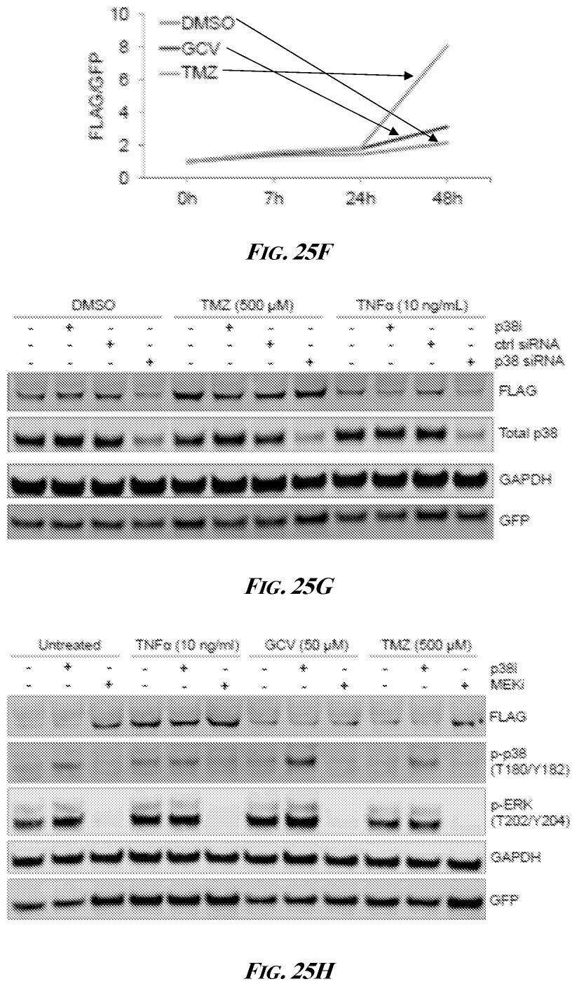

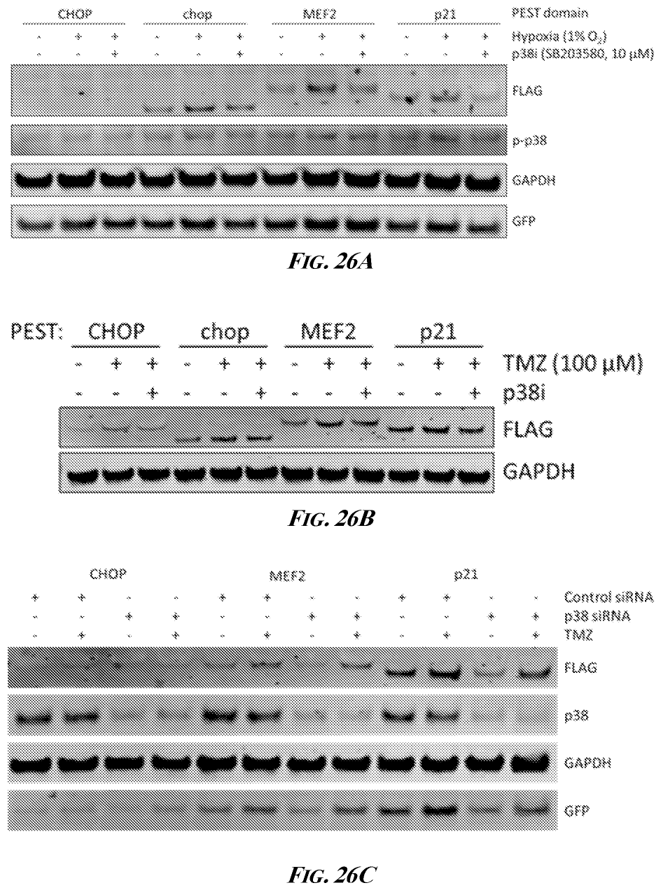

Referring to FIG. 25A, four designs tested included peptide sequences from the MAPK targets CHOP, MEF2A, p21, and FRA1. Schematics of design are shown, with the predicted PEST domains and relative position of serine residues demonstrated in the literature to be phosphorylated by p38 (“P” in circles). Sequences were taken from proteins primarily phosphorylated by p38, while other domains were taken from the original FRA 1-PEST PEST design. The ERK recognition sequence was removed from these designs to improve specificity toward p38. G816 cells (glioma-initiating cell line) expressing each of the four HSVtk constructs were grown in 21% oxygen or 1% oxygen (hypoxia), with or without SB 203580 (p38 inhibitor, 10 M for two days. Hypoxic induction of p38 phosphorylation promoted protein stabilization, which was abrogated with the addition of a p38 inhibitor to varying degrees for each construct.

Referring to FIG. 25B, immunofluorescence microscopy images demonstrate nuclear localization of FLAG signal and varying baseline abundance.

U87 cells (differentiated glioblastoma cell line) were transduced with retrovirus carrying each of the four suicide genes. Nuclear-localized FLAG is only present in cells with high GFP expression and relatively high nuclear p38 (solid white arrows). In cells with insufficient GFP or low levels of nuclear p38, FLAG abundance is similarly reduced (white-outlined arrows), indicating the importance of both gene transcription and p38 phosphorylation on HSVtk abundance. Further, baseline FLAG abundance is dependent on the design of each PEST domain. Scale bars=100 m.

Referring to FIGS. 25E and 25F, G816-FLAG-NLS-HSVtk-MEF2A cells were treated with DMSO (vehicle), GCV (50 μM), or TMZTMZ (500 μM) for up to 48 hours. Both GCV and TMZ treatment increased FLAG abundance after 48 hours of treatment, relative to vehicle-treated samples. Interestingly, FLAG abundance did increase in the vehicle-treated condition, which could be a result of increasing cellular confluence or the use of FGFb in the cell culture medium, both of which can promote p38 activity.

Referring to FIG. 25G, G816-FLAG-NLS-HSVtk-MEF2A cells were transduced with a control siRNA or an siRNA against p38 using Lipofectamine RNAiMAX. Cells were then treated with no inhibitor or SB 203580 (p38 inhibitor, 10 μM) and DMSO (vehicle), TMZ, or TNFα (tumor necrosis factor α) for two days. In the cases of DMSO- and TNFα-treated cells, knockdown of p38 reduced FLAG abundance, indicating that p38 is necessary to stabilize the protein in these contexts. However, knockdown of p38 in the TMZ-treated cells increased FLAG abundance, indicating another kinase may be involved in PEST phosphorylation.

Referring to FIG. 25H, G816-FLAG-NLS-HSVtk-MEF2A cells were treated with DMSO (vehicle), SB 203580 (p38 inhibitor, 10 μM), or trametinib (MEK inhibitor, inhibitor, 50 nM), in addition to TNFα, GCV, or TMZ for two days. FLAG expression was increased by the p38-inducing treatments TNFα, GCV, and TMZ was attenuated in some cases by p38 inhibition. Interestingly, MEK inhibition also promoted PEST stabilization, indicating that there may be a compensatory MAPK signaling mechanism.

In some embodiments of the presently disclosed subject matter, the peptide domain that is stabilized when phosphorylated by p38 MAP kinase activity and/or JNK activity in a target cell and/or tissue comprises a peptide domain as set forth in any of FIGS. 15-20, or a fragment thereof, or a substantially homologous amino acid sequence thereto, or a substantially homologous amino acid sequence to a fragment thereof. In some embodiments, the peptide domain comprises amino acids 266-282 of SEQ ID NO. 11, amino acids 444-462 of SEQ ID NO. 11, amino acids 2-101 of SEQ ID NO. 13, amino acids 50-101 of SEQ ID NO. 13, amino acids 122-140 of SEQ ID NO. 16, amino acids 163-271 of SEQ ID NO. 18, amino acids 180-196 of SEQ ID NO. 18, or amino acids 251-270 of SEQ ID NO. 18.

### III.A. Representative Vectors

Thus, in some embodiments, the presently disclosed subject matter provides a vector comprising a first nucleic acid sequence encoding a promoter operably linked to each of a second nucleic acid sequence encoding a therapeutic polypeptide, optionally a third nucleic acid sequence encoding a nuclear localization sequence (NLS), and a fourth nucleic acid sequence encoding a peptide domain that is stabilized when phosphorylated by when phosphorylated by the kinase activity, such as but not limited to extracellular regulated kinase (ERK) activity or a kinase activity associated with a target cell and/or undergoing a stress response, such as p38 MAP kinase activity. Thus, the order of encoded peptides is changeable and, in some embodiments, not all peptides are included, such as in the fusion protein. For example, in some embodiments the NLS is not included, such as in a fusion protein embodiment.

In some embodiments, a “stress response” refers to group of signaling and other biochemical processes that occur when cells encounter toxic agents, including but not limited to DNA damaging agents such as temozolomide chemotherapy. The p38 MAPK (mitogen activated protein kinase) pathway is one such signaling pathway.

By the term “therapeutic polypeptide” it is meant a polypeptide that can have any sort of therapeutic effect in a cell and/or of interest for selective targeting through a peptide domain that is stabilized when phosphorylated by a kinase in a target cell and/or. In some embodiments, the target cell and/or is a target cell and/or undergoing a stress response. This strategy, in some embodiments an Erk-regulated strategy or a p38 MAP kinase-regulated strategy, can be employed for cancer-selective expression of a number of therapeutic polypeptides, including but not limited to the following: 1) a suicide gene approach with activation of a prodrug; 2) expression of a secreted toxic protein, such as TRAIL; and 3) expression of a secreted immune cytokine or immune danger signal.

Thus, in some embodiments, the presently disclosed subject matter provides a strategy, including methods and compositions, that yields tissue-selective (e. g., kinase-based, e. g. Erk-based or p38 MAP kinase-based, e. g., cancer-selective) expression of any protein, i. e., a therapeutic polypeptide, for treatment of a disease or disorder in a subject, such as a disease or disorder associated with kinase activity, such as ERK-activity or p38 MAP activity, such as cancer, a disease or disorder characterized by the presence of a stress response, such as cancer, such as a cancer tissue undergoing chemotherapy or other therapy.

In some embodiments, the vector comprises a first nucleic acid sequence encoding a promoter operably linked to a second nucleic acid sequence encoding a fusion protein comprising the therapeutic polypeptide and the peptide domain that is stabilized when phosphorylated by kinase activity, such as but not limited to a kinase activity associated with a target cell and/or undergoing a stress response, such as p38 MAP kinase activity, or extracellular regulated kinase (ERK) activity or p38 MAP kinase activity. In some embodiments, the fusion protein comprises an NLS. In some embodiments, the nucleic acid sequence encoding the fusion protein comprises (a) a nucleic acid sequence encoding the therapeutic polypeptide, (b) a nucleic acid sequence encoding the NLS, and (c) a nucleic acid sequence encoding the peptide domain that is stabilized when phosphorylated by kinase activity, such as but not limited to a kinase activity associated with a target cell and/or undergoing a stress response, such as p38 MAP kinase activity, or extracellular regulated kinase (ERK) activity or p38 MAP kinase activity. In some embodiments, the nucleic acid sequences of (a), (b), and (c) are fused in frame such that the NLS is at an N-terminus of the fusion protein and the peptide domain that is stabilized when phosphorylated by kinase activity, such as but not limited to a kinase activity associated with a target cell and/or undergoing a stress response such as p38 MAP kinase activity or extracellular regulated kinase (ERK) activity or p38 MAP kinase activity is at a C-terminus of the fusion protein.

In some embodiments, the therapeutic polypeptide comprises a Herpes simplex virus thymidine kinase (HSVtk) polypeptide or a yeast cytosine deaminase polypeptide. The therapeutic polypeptide is also referred to herein as a suicide gene product or as being encoded by a suicide gene.

In some embodiments, the HSVtk polypeptide comprises a polypeptide selected from the group consisting of a polypeptide having an amino acid sequence as set forth in SEQ ID NO:1, a homolog thereof, a fragment thereof, or a homolog of the fragment thereof.

In some embodiments, the HSVtk polypeptide comprises a polypeptide selected from the group consisting of a HSVtk polypeptide a polypeptide having an amino acid sequence having 95% homology to SEQ ID NO:1, and a fragment thereof. In some embodiments, the HSVtk polypeptide comprises a polypeptide selected from the group consisting of a polypeptide having an amino acid sequence as set forth in SEQ ID NO:1, a fragment thereof, a polypeptide having an amino acid sequence having 95% homology to SEQ ID NO:1, and a fragment thereof. In some embodiments, the amino acid sequence of the polypeptide comprises least one modification selected from the group consisting of an amino acid deletion, an amino acid addition, an amino acid substitution, and combinations thereof.

In some embodiments, the nucleic acid sequence is selected from the group consisting of (a) a nucleic acid sequence encoding a polypeptide having an amino acid sequence as set forth in SEQ ID NO: 1, or a fragment or homolog thereof, (b) a nucleic acid sequence as set forth in SEQ ID NO: 2, or its complementary strands; (c) a homologous nucleic acid sequence to a nucleic acid sequence as set forth in SEQ ID NO: 2, and which encodes a HSVtk polypeptide; and (d) a nucleic acid sequence differing from an isolated nucleic acid molecule of (a), (b), or (c) above due to degeneracy of the genetic code, and which encodes a HSVtk polypeptide encoded by the isolated nucleic acid molecule of (a), (b), or (c) above.

In some embodiments, the nucleic acid sequence is selected from the group consisting of: (a) a nucleic acid sequence encoding a polypeptide comprising an amino acid sequence as set forth in SEQ ID NO: 1; a fragment thereof, a polypeptide having an amino acid sequence having 95% homology to SEQ ID NO: 1, and a fragment thereof, (b) a nucleic acid sequence as set forth in SEQ ID NO: 2, or its complementary strands; (c) a nucleic acid sequence having 95% homology to a nucleic acid sequence as set forth in SEQ ID NO: 2, and which encodes a HSVtk polypeptide; and (d) a nucleic acid sequence differing from an isolated nucleic acid molecule of (a), (b), or (c) above due to degeneracy of the genetic code, and which encodes a HSVtk polypeptide encoded by the isolated nucleic acid molecule of (a), (b), or (c) above.

Other representative HSVtk sequences would be apparent to one of ordinary skill in the art upon a review of the instant disclosure. These sequences include but are not limited to those sequences disclosed in GenBank Accession Numbers: Q9QNF71, P03176-1, AAP13943.1, AAA45811.1, J04327.1 (included as SEQ ID NO:2 in the present Sequence Listing) AB009254.2, AB032890.1, AB032887.1, and AB032886.1.

In some embodiments, the yeast cytosine deaminase (yCD) polypeptide comprises a polypeptide selected from the group consisting of a polypeptide having an amino acid sequence as set forth in SEQ ID NO: 5, a homolog thereof, a fragment thereof, or a homolog of the fragment thereof. In some embodiments, the yCD polypeptide comprises a polypeptide selected from the group consisting of a polypeptide having an amino acid sequence as set forth in SEQ ID NO: 5, a fragment thereof, a polypeptide having an amino acid sequence having 95% homology to SEQ ID NO: 5, and a fragment thereof. In some embodiments, the amino acid sequence of the polypeptide comprises least one modification selected from the group consisting of an amino acid deletion, an amino acid addition, an amino acid substitution, and combinations thereof.

In some embodiments, the nucleic acid sequence is selected from the group consisting of (a) a nucleic acid sequence encoding a polypeptide having an amino acid sequence as set forth in SEQ ID NO: 5, or a fragment or homolog thereof, (b) a nucleic acid sequence as set forth in SEQ ID NO: 6, or its complementary strands; (c) a homologous nucleic acid sequence to a nucleic acid sequence as set forth in SEQ ID NO: 6, and which encodes a yCD polypeptide; and (d) a nucleic acid sequence differing from an isolated nucleic acid molecule of (a), (b), or (c) above due to degeneracy of the genetic code, and which encodes a yCD polypeptide encoded by the isolated nucleic acid molecule of (a), (b), or (c) above.

In some embodiments, the nucleic acid sequence is selected from the group consisting of: (a) a nucleic acid sequence encoding a polypeptide comprising an amino acid sequence as set forth in SEQ ID NO: 5; a fragment thereof, a polypeptide having an amino acid sequence having 95% homology to SEQ ID NO: 5, and a fragment thereof, (b) a nucleic acid sequence as set forth in SEQ ID NO:6, or its complementary strands; (c) a nucleic acid sequence having 95% homology to a nucleic acid sequence as set forth in SEQ ID NO:6, and which encodes a yCD polypeptide; and (d) a nucleic acid sequence differing from an isolated nucleic acid molecule of (a), (b), or (c) above due to degeneracy of the genetic code, and which encodes a yCD polypeptide encoded by the isolated nucleic acid molecule of (a), (b), or (c) above.

In some embodiments, the peptide domain that is stabilized when phosphorylated by kinase activity, such as but not limited to a extracellular regulated kinase (ERK) activity or an activity related to kinase activity in an environment associated with a stress response, such as a p38 MAP kinase activity, comprises a Fra1-based integrative reporter (FIRE) polypeptide, referred to herein using the terms “FIRE”, “FIRE polypeptide”, “FIRE peptide domain”, “FIRE peptide”, and “FIRE domain”, interchangeably. In some embodiments, the 30 FTRE polypeptide comprises a polypeptide selected from the group consisting of a polypeptide having an amino acid sequence as set forth in SEQ ID NO:4, a fragment thereof, a homolog thereof, a fragment thereof, or a homolog of the fragment thereof. In some embodiments, the FIRE polypeptide comprises a polypeptide selected from the group consisting of a polypeptide having an amino acid sequence as set forth in SEQ ID NO:4, a fragment thereof, a polypeptide having an amino acid sequence having 95% homology to SEQ ID NO:4, and a fragment thereof. Other representative FIRE sequences would be apparent to one of ordinary skill in the art upon a review of the instant disclosure. These sequences include but are not limited to those sequences disclosed in GenBank Accession Numbers HGNC:13718; BC016648. (included as SEQ ID NO: 3 in the present Sequence Listing), CR542278.1, CR542257.1, NM 005438.4, NM_001300857.1, NM_001300856.1, NM_001300855.1. In some embodiments, the FIRE polypeptide comprises amino acids 163-271 (also referred to herein as a PEST domain) of SEQ ID NO:4, or fragment or homolog thereof. In some embodiments, the amino acid sequence of the polypeptide comprises least one modification selected from the group consisting of an amino acid deletion, an amino acid addition, an amino acid substitution, and combinations thereof.

In some embodiments, the nucleic acid sequence is selected from the group consisting of (a) a nucleic acid sequence encoding a polypeptide having an amino acid sequence as set forth in SEQ ID NO: 4, or a fragment or homolog thereof, (b) a nucleic acid sequence as set forth in SEQ ID NO: 3, or its complementary strands; (c) a homologous nucleic acid sequence to a nucleic acid sequence as set forth in SEQ ID NO: 3, and which encodes a FIRE polypeptide; and (d) a nucleic acid sequence differing from an isolated nucleic acid molecule of (a), (b), or (c) above due to degeneracy of the genetic code, and 20 which encodes a FRE polypeptide encoded by the isolated nucleic acid molecule of (a), (b), or (c) above.

In some embodiments, the nucleic acid sequence is selected from the group consisting of: (a) a nucleic acid sequence encoding a polypeptide comprising an amino acid sequence as set forth in SEQ ID NO: 4; a fragment thereof, a polypeptide having an amino acid sequence having 95% homology to SEQ ID NO: 4, and a fragment thereof, (b) a nucleic acid sequence as set forth in SEQ ID NO: 3, or its complementary strands; (c) a nucleic acid sequence having 95% homology to a nucleic acid sequence as set forth in SEQ ID NO: 3, and which encodes a FIRE polypeptide; and (d) a nucleic acid sequence differing from an isolated nucleic acid molecule of (a), (b), or (c) above due to degeneracy of the genetic code, and which encodes a FTRE polypeptide encoded by the isolated nucleic acid molecule of (a), (b), or (c) above.

Any suitable NLS as would be apparent to one of ordinary skill in the art upon in a review of the instant disclosure can be employed. In some embodiments, a nuclear localization sequence is MAPKKKRK (SEQ ID NO: 7), or a homolog or fragment thereof. In some embodiments, an NLS is PKKKRKV (SEQ ID NO: 8), or a homolog or fragment thereof. In some embodiments, an NLS is KRPAATKKAGQAKKKK (SEQ ID NO: 9), or a homolog or fragment thereof. In some embodiments, an NLS is PAAKRVKLD (SEQ ID NO: 10), or a homolog or fragment thereof (see Makkerh et al., 1996). In some embodiments, the amino acid sequence of the polypeptide comprises least one modification selected from the group consisting of an amino acid deletion, an amino acid addition, an amino acid substitution, and combinations thereof.

FIGS. 15 through 20 present representative isolated amino acid sequences and nucleic acid sequences encoding the same that are employed with p38 MAP kinase embodiments as disclosed herein. Also provided are substantially identical amino acid sequences and substantially identical nucleic acid sequences to the amino acid sequences and nucleic acid sequences disclosed in FIGS. 15-20. Also provided are fragments of the amino acid sequences and nucleic acid sequences disclosed in FIGS. 15-20 and substantially identical amino acid sequences and substantially identical nucleic acid sequences to the fragments. Also provided are complementary strands of any of these nucleic acid sequences and a nucleic acid sequence differing from any of the nucleic acids due to degeneracy of the genetic code, and which encodes an amino acid sequence encoded by the isolated nucleic acid molecule.

It is recognized that the gene of interest may be modified by any methods known in the art. For example, the gene may be placed under the control of heterologous regulatory regions, including the use of viral promoters, neoplastic cell or tumor specific promoters or control elements. In this manner, the gene product is further targeted to specific cell types. Methods for construction of such expression vectors are described herein and are known in the art.

In some embodiments, the presently disclosed subject matter provides compositions and methods encompassing an oncogene activity-dependent vector system (e. g., oncogene activity-dependent suicide gene vector system) useful for selective targeting of cancer cells. In some embodiments, selective targeting of cancer cells undergoing a stress response is provided, using a p38 MAP kinase approach. The stress response can be caused by a treatment, such as but not limited to treatment with an anti-cancer drug. In some embodiments, the presently disclosed subject matter provides compositions and methods useful for killing cells using a suicide gene upon prodrug administration.

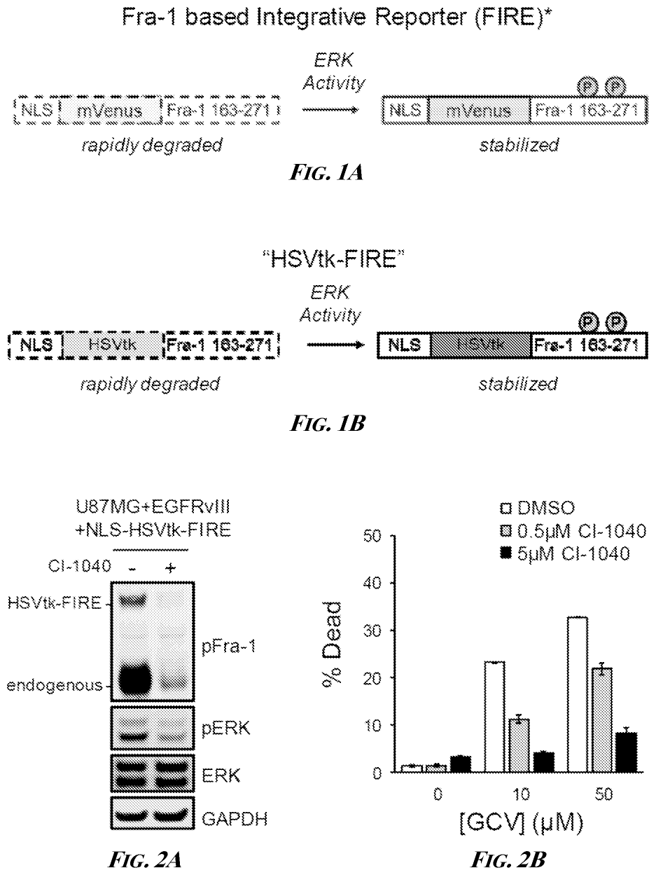

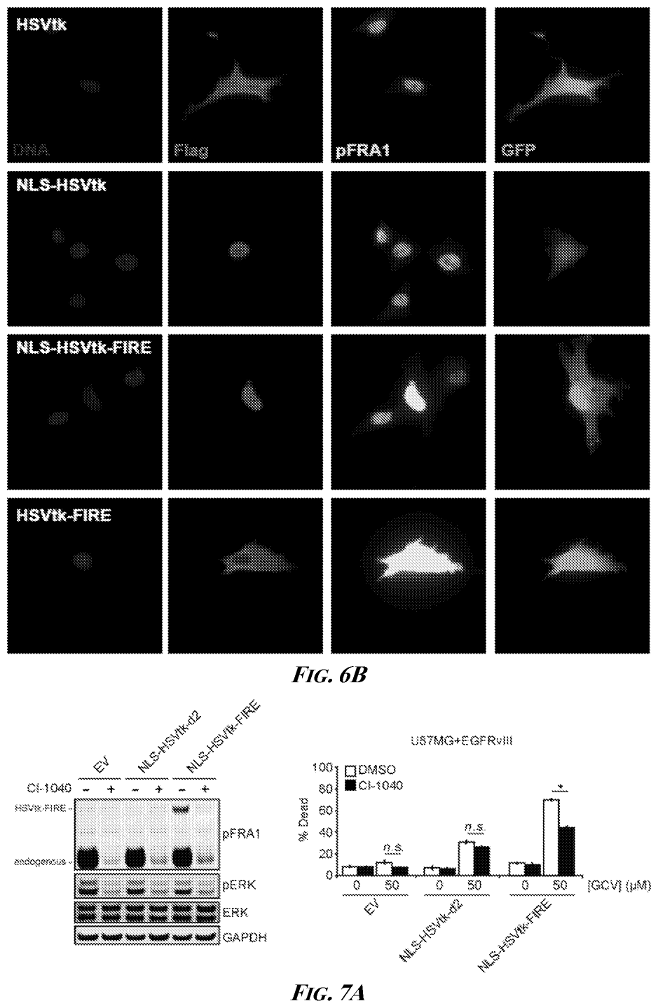

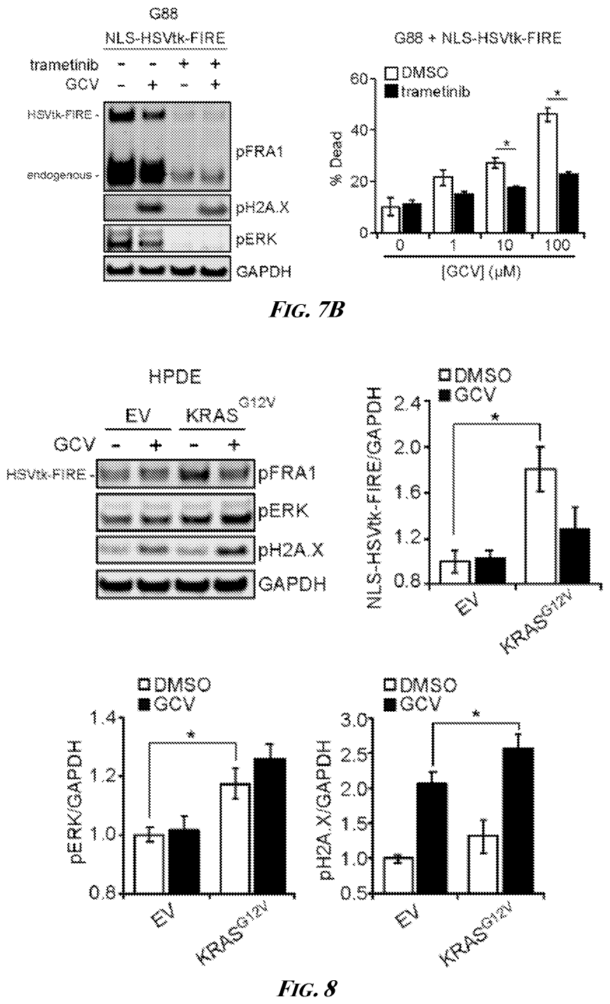

In some embodiments, the presently disclosed subject matter provides a novel ERK-stabilized or a novel p38 MAP kinase-stabilized suicide gene, which converts a prodrug such as ganciclovir (GCV) into a toxic product through the expression of the therapeutic polypeptide such as Herpes simplex virus thymidine kinase (HSVtk) protein, where the therapeutic polypeptide has been fused to a nuclear localization sequence (NLS) and a peptide domain (such as a Fra1 domain) that is stabilized when phosphorylated by active kinase, such as ERK or p38 MAP kinase which is preferentially shuttled to the cell nucleus (FIG. 1B, Example 1, and FIGS. 5A and 6A). In some embodiments, a gene/protein of the presently disclosed subject matter is HSVtk-FIRE.

ERK-dependent or p38 MAP kinase-dependent dependent suicide gene therapy have advantages over other treatments, such as the use of drugs, because, for example, ERK-dependent or p38 MAP kinase-dependent suicide gene therapy turns a cell survival mechanism into a lethal vulnerability, it becomes even more effective as cancer cells attempt to resist (feedback), it targets cancer cells but not healthy cells (selectivity), and it can be used in combination with other therapies.

The current methods provide improvements over the art with better gene delivery efficiency, less prodrug (e. g. GCV) needed (decreased dose), and selectivity.

In some embodiments, the presently disclosed subject matter provides compositions and methods as part of a suicide gene/prodrug strategy, e. g. an HSVtk/GCV prodrug strategy, which selectively targets cells with aberrant ERK activity, e. g., elevated ERK activity. In some embodiments, the cancer with aberrant ERK activity is glioblastoma. In some embodiments, the compositions and methods of the presently disclosed subject matter are useful treatment for cancer cells with up-regulated ERK due to high Ras or high RAF activity.

In some embodiments, the presently disclosed subject matter provides compositions and methods as part of a suicide gene/prodrug strategy, e. g. an HSVtk/GCV prodrug strategy employing a p38 MAP kinase, which selectively targets cells undergoing a stress response, such as a cell exposed to a chemotherapeutic agent or other stress response. In some embodiments, the cancer undergoing a stress response is glioblastoma. In some embodiments, the compositions and methods of the presently disclosed subject matter are useful treatment for cancer cells being treated with a treatment that causes a stress response, such as but not limited to treatment with a chemotherapeutic agent.

In some embodiments, the presently disclosed subject matter provides compositions and methods useful as an ERK-dependent and p38 MAP kinase-dependent suicide gene therapy for cancer. In some embodiments, the cancer is GBM.

In some embodiments, the presently disclosed subject matter provides compositions and methods useful for overcoming ERK-mediated and other resistance mechanisms which cancer cells commonly utilize to evade kinase inhibition.

In some embodiments, administration of the vector (e. g., HSVtk-FIRE) is useful for overcoming ERK mediated and other resistance mechanisms.

In some embodiments, the peptide domain that is stabilized when phosphorylated by ERK (e. g., Fra-1 domain) or by a p38 MAP kinase creates an inescapable feedback loop in the target cells and the prodrug (e. g., modified GCV (monophosphate form)) is more effective in killing the cells.

In some embodiments, the vector and prodrug (e. g., HSVtk-FIRE and GCV) are administered to treat cancer susceptible to such treatment along with the use of at least one additional therapeutic agent. In some embodiments, the additional therapeutic agent is an anti-cancer drug, radiation, or a combination thereof.

In some embodiments, the presently disclosed subject matter provides a vector system. In some embodiments, the vector system comprises an ERK-stabilized suicide gene or a p38 MAP kinase-stabilized suicide gene, which encodes a therapeutic polypeptide that converts a prodrug (e. g., ganciclovir (GCV)) into a toxic product through the expression of the therapeutic polypeptide (e. g, Herpes simplex virus thymidine kinase (HSVtk) protein). In some embodiments, the vector system provides for the fusion of the therapeutic polypeptide (e. g., HSVtk protein) to a nuclear localization sequence (NLS) and a peptide domain (e. g., Fra1 domain) that is stabilized when phosphorylated by active ERK or a peptide domain that is stabilized by active p38 MAP kinase, which is preferentially shuttled to the cell nucleus. In a particular embodiment, this fusion protein is referred to as HSVtk-FIRE, where FIRE stands for Fra1-based integrative reporter. The fusion of HSVtk with the NLS and Fra1 domains creates an ERK-stabilized HSVtk protein that provides for ERK-specific killing of cancer cells.

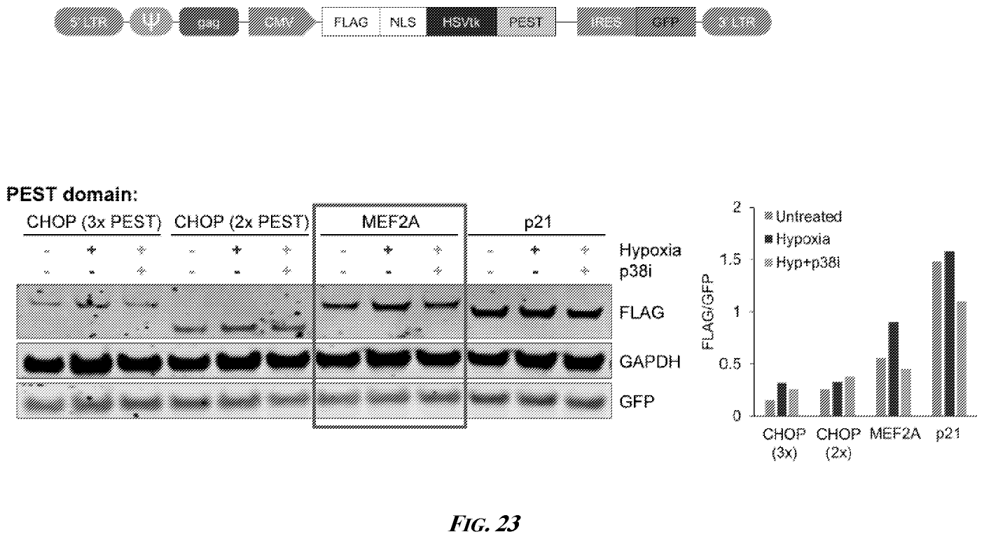

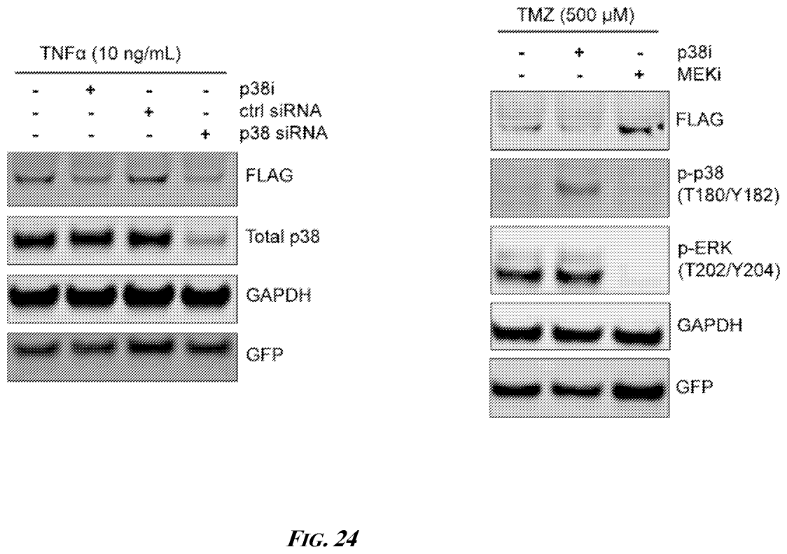

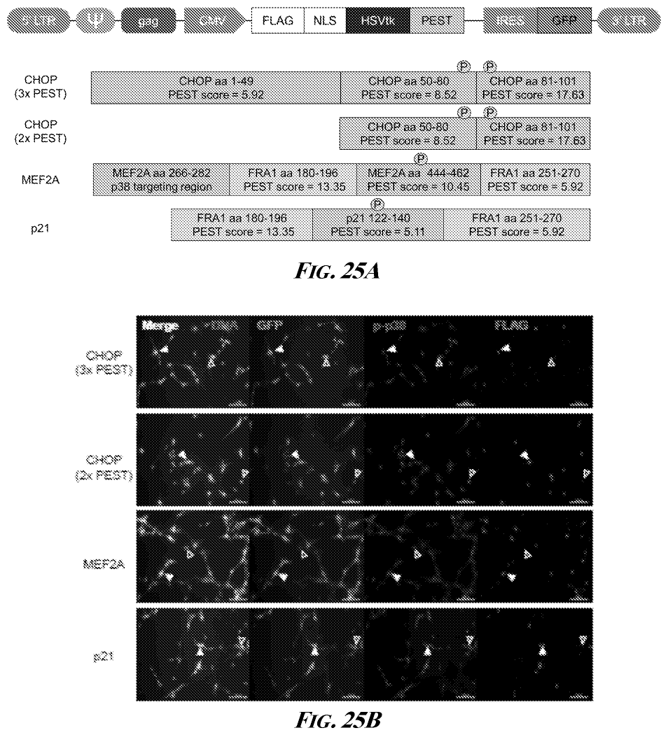

Suicide gene therapies involve the transduction of cancer cells with genes that enable the conversion of an innocuous prodrug into a toxic product. We have designed a novel approach based on the activity of the p38 MAP kinase pathway for controlling the expression of the herpes simplex virus thymidine kinase (HSVtk), a common suicide gene product that converts the prodrug ganciclovir into a toxic phosphorylated form. Because p38 is activated in response to chemotherapy-induced DNA damage and other cell stresses, our design could potentially combine synergistically with chemotherapy to create a positive feedback loop that leads to enhanced tumor cell killing. Novel combination therapy approaches are desperately needed for cancers such as glioblastoma, which is notoriously resistant even to harsh chemotherapeutic regimens. In some embodiments, HSVtk was fused with peptide sequences whose turnover was predicted to be slowed when phosphorylated by p38. Four such peptides were created using PEST domains (sequences rich in proline, glutamic acid, serine, and threonine that regulate protein turnover) from the MAP kinase substrates CHOP, MEF2A, p21, and FRA1 as starting points. See FIGS. 15-20. Retroviral expression vectors encoding these novel suicide genes were cloned and used to transduce a human glioma-initiating cell line. The selectivity of these constructs for p38-dependent cell killing and the cooperativity of these novel suicide genes with temozolomide chemotherapy was observed. See FIGS. 21-25. More broadly, in some embodiments, the presently disclosed subject matter demonstrated the potential utility of post-translational control of suicide gene activity as an alternative to transcriptional regulation.

In some embodiments, integrating viral vectors are chosen for gene therapy because they offer efficient transduction and consistent long-term gene expression.

### III.B. Representative Sequences

SEQ ID NO: 1: Human Herpes alphaherpesvirus 1 thymidine kinase amino acid
sequence. See e.g., Accession No. AAA45811. 1 of the GENBANK ® biosequence
database.
MASYPCHQHASAFDQAARSRGHSNRRTALRPRRQQEATEVRLEQKMPTLLRVYIDGPHGM
GKTTTTQLLVALGSRDDIVYVPEPMTYWQVLGASETIANIYTTQHRLDQGEISAGDAAVV
MTSAQITMGMPYAVTDAVLAPHVGGEAGSSHAPPPALTLIFDRHPIAALLCYPAARYLMG
SMTPQAVLAFVALIPPTLPGTNIVLGALPEDRHIDRLAKRQRPGERLDLAMLAAIRRVYG
LLANTVRYLQGGGSWWEDWGQLSGTAVPPQGAEPQSNAGPRPHIGDTLFTLFRAPELLAP
NGDLYNVFAWALDVLAKRLRPMHVFILDYDQSPAGCRDALLQLTSGMVQTHVTTPGSIPT
ICDLARTFAREMGEAN
SEQ ID NO: 2: HSVtk coding sequence. See e.g., Accession Nos. MK896152.1,
AB009254.2, AB032890.1, AB032887.1, AB032886.1, and J04327.1 of the
GENBANK ® biosequence database.
atggcttcgtacccctgccatcaacacgcgtctgcgttcgaccaggctgcgcgttctcgc
ggccatagcaaccgacgtacggcgttgcgccctcgccggcagcaagaagccacggaagtc
cgcctggagcagaaaatgcccacgctactgcgggtttatatagacggtcctcacgggatg
gggaaaaccaccaccacgcaactgctggtggccctgggttcgcgcgacgatatcgtctac
gtacccgagccgatgacttactggcaggtgctgggggcttccgagacaatcgcgaacatc
tacaccacacaacaccgcctcgaccagggtgagatatcggccggggacgcggcggtggta
atgacaagcgcccagataacaatgggcatgccttatgccgtgaccgacgccgttctggct
cctcatatcgggggggaggctgggagctcacatgccccgcccccggccctcaccctcatc
ttcgaccgccatcccatcgccgccctcctgtgctacccggccgcgcgataccttatgggc
agcatgaccccccaggccgtgctggcgttcgtggccctcatcccgccgaccttgcccggc
acaaacatcgtgttgggggcccttccggaggacagacacatcgaccgcctggccaaacgc
cagcgccccggcgagcggcttgacctggctatgctggccgcgattcgccgcgtttacggg
ctgcttgccaatacggtgcggtatctgcagggcggcgggtcgtggcgggaggattgggga
cagctttcggggacggccgtgccgccccagggtgccgagccccagagcaacgcgggccca
cgaccccatatcggggacacgttatttaccctgtttcgggcccccgagttgctggccccc
aacggcgacctgtacaacgtgtttgcctgggccttggacgtcttggccaaacgcctccgt
cccatgcacgtctttatcctggattacgaccaatcgcccgccggctgccgggacgccctg
ctgcaacttacctccgggatggtccagacccacgtcaccaccccaggctccataccgacg
atctgcgacctggcgcgcacgtttgcccgggagatgggggaggctaactga
SEQ ID NO.: 3: Human FOSL1/FRA1 (FOS-like antigen 1/FOS-related antigen)
nucleic acid sequence encoding a FIRE polypeptide. See e.g., HGNC: 13718 and Accession
Nos. CR542278.1, CR542257.1, NM_005438.5, NM_001300844.2, NM_001300855.2,
NM_001300856.2, and NM_001300857.2 of the GENBANK ® biosequence database.
cagaacccagcagccgtgtaccccgcagagccgccagccccgggcatgttccgagacttc
ggggaacccggcccgagctccgggaacggcggcgggtacggcggccccgcgcagcccccg
gccgcagcgcaggcagcccagcagaagttccacctggtgccaagcatcaacaccatgagt
ggcagtcaggagctgcagtggatggtacagcctcatttcctggggcccagcagttacccc
aggcctctgacctaccctcagtacagccccccacaaccccggccaggagtcatccgggcc
ctggggccgcctccaggggtacgtcgaaggccttgtgaacagatcagcccggaggaagag
gagcgccgccgagtaaggcgcgagcggaacaagctggctgcggccaagtgcaggaaccgg
aggaaggaactgaccgacttcctgcaggcggagactgacaaactggaagatgagaaatct
gggctgcagcgagagattgaggagctgcagaagcagaaggagcgcctagagctggtgctg
gaagcccaccgacccatctgcaaaatcccggaaggagccaaggagggggacacaggcagt
accagtggcaccagcagcccaccagccccctgccgccctgtaccttgtatctccctttcc
ccagggcctgtgcttgaacctgaggcactgcacacccccacactcatgaccacaccctcc
ctaactcctttcacccccagcctggtcttcacctaccccagcactcctgagccttgtgcc
tcagctcatcgcaagagtagcagcagcagcggagacccatcctctgacccccttggctct
ccaaccctcctcgctttgtgaggcgcctgagccctactccctgcagatgccaccctagcc
aatgtctcctccccttcccccaccggtccagctggcctggacagtatcccacatccaact
ccagcaacttcttctccatccctctaatgagactgaccatattgtgcttcacagtagagc
cagcttggggccaccaaagctgcccactgtttctcttgagctggcctctctagcacaatt
tgcactaaatcagagacaaaatatttcccatttgtgccagaggaatcctggcagcccaga
gactttgtagatccttagaggtcctctggagccctaaccccttccagatcactgccacac
tctccatcaccctcttcctgtgatccacccaaccctatctcctgacagaaggtgccactt
tacccacctagaacactaactcaccagccccactgccagcagcagcaggtgattggacca
ggccattctgccgccccctcctgaaccgcacagctcaggaggcgcccttggcttctgtga
tgagctgatctgcggatctcagctttgagaagccttcagctccagggaatccaagcctcc
acagcgagggcagctgctatttattttcctaaagagagtatttttatacaaacctaccaa
aatggaataaaaggcttgaagctgtgaaaaaaaaaaaaaaaaaaaaaaaaa
SEQ ID NO: 4: Human FIRE amino acid sequence. See e.g. , Accession No.
NP_005429.1.
MFRDFGEPGPSSGNGGGYGGPAQPPAAAQAAQQKFHLVPSINTMSGSQELQWMVQPHFLG
PSSYPRPLTYPQYSPPQPRPGVIRALGPPPGVRRRPCEQISPEEEERRRVRRERNKLAAA
KCRNRRKELTDFLQAETDKLEDEKSGLQREIEELQKQKERLELVLEAHRPICKIPEGAKE
GDTGSTSGTSSPPAPCRPVPCISLSPGPVLEPEALHTPTLMTTPSLTPFTPSLVFTYPST
PEPCASAHRKSSSSSGDPSSDPLGSPTLLAL
SEQ ID NO: 5: Yeast cytosine deaminase amino acid sequence. See e.g. ,
Accesstion No. NP_015387.1 of the GENBANK ® biosequence database.
MVTGGMASKWDQKGMDIAYEEAALGYKEGGVPIGGCLINNKDGSVLGRGHNMRFQKGSAT
LHGEISTLENCGRLEGKVYKDTTLYTTLSPCDMCTGAIIMYGIPRCVVGENVNFKSKGEK
YLQTRGHEVVVVDDERCKKIMKQFIDERPQDWFEDIGE
SEQ ID NO: 6: Yeast cytosine deaminase coding sequence. See e.g. , Accession
No. NM_001184159.1 of the GENBANK ® biosequence database.
atggtgacagggggaatggcaagcaagtgggatcagaagggtatggacattgcctatgag
gaggcggccttaggttacaaagagggtggtgttcctattggcggatgtcttatcaataac
aaagacggaagtgttctcggtcgtggtcacaacatgagatttcaaaagggatccgccaca
ctacatggtgagatctccactttggaaaactgtgggagattagagggcaaagtgtacaaa
gataccactttgtatacgacgctgtctccatgcgacatgtgtacaggtgccatcatcatg
tatggtattccacgctgtgttgtcggtgagaacgttaatttcaaaagtaagggcgagaaa
tatttacaaactagaggtcacgaggttgttgttgttgacgatgagaggtgtaaaaagatc
atgaaacaatttatcgatgaaagacctcaggattggtttgaagatattggtgag

FIGS. 15 through 20 present representative amino acid sequences and nucleic acid sequences employed with p38 MAP kinase embodiments as disclosed herein. Also provided are substantially identical amino acid sequences and substantially identical nucleic acid sequences to the amino acid sequences and nucleic acid sequences disclosed in FIGS. 15-20. Also, provided are fragments of the amino acid sequences and nucleic acid sequences disclosed in FIGS. 15-20 and substantially identical amino acid sequences and substantially identical nucleic acid sequences to the fragments.

In further embodiments, approaches for delivery enhance the impact and applicability of this overall strategy. In some embodiments, the vector can comprise a cell-penetrating peptide, such as an Antennapedia cell-penetrating peptide, which can facilitate transport of peptides and whole proteins through the cell membrane. By way of particular but non-limiting example, an Antennapedia cell-penetrating peptide is fused to to the N-terminus of HSVtk-FIRE. In vitro testing of this fusion protein is performed in GBM cells to ensure that it is able to cross the cell membrane and trigger cell death with ganciclovir exposure. If so, the local CED administration of the fusion protein is tested in the models disclosed herein, with a single dose administered seven days after GIC infusion. Daily ganciclovir dosing begins the day of protein infusion. In addition, the use of focused ultrasound (FUS) is leveraged to promote convection-enhanced delivery (CED) of a construct in accordance with the presently disclosed subject matter, such as but not limited to HSVtk-FIRE, in viral vectors or liposomes.

The polypeptides and/or fusion products of the presently disclosed subject matter can be varied by using biologically active fragments of the sequences as described herein. See also Albeck et al., 2013, the entirety of which is incorporated by reference herein. Thus, the presently disclosed subject matter provides polypeptides and biologically active fragments and homologs thereof as well as methods for preparing and testing new polypeptides for the properties disclosed herein. In some embodiments, the fragments are mammalian. In some embodiments, the fragments are human.

In some embodiments, a polypeptide or biologically active fragment or homolog thereof is useful for treating a disease or disorder as disclosed herein.

In some embodiments, the presently disclosed subject matter uses a biologically active polypeptide or biologically active fragment or homolog thereof. In some embodiments, the isolated polypeptide comprises a mammalian molecule at least about 30% homologous to a polypeptide having the amino acid sequence of at least one of the sequences disclosed herein. In some embodiments, the isolated polypeptide is at least about 35% homologous, more in some embodiments, about 40% homologous, more in some embodiments, about 45% homologous, in some embodiments, about 50% homologous, more in some embodiments, about 55% homologous, in some embodiments, about 60% homologous, more in some embodiments, about 65% homologous, in some embodiments, more in some embodiments, about 70% homologous, more in some embodiments, about 75% homologous, in some embodiments, about 80% homologous, more in some embodiments, about 85% homologous, more in some embodiments, about 90% homologous, in some embodiments, about 95% homologous, more in some embodiments, about 96% homologous, more in some embodiments, about 97% homologous, more in some embodiments, about 98% homologous, and most in some embodiments, about 99% homologous to at least one of the peptide sequences disclosed herein.

The presently disclosed subject matter further encompasses modification of the polypeptides and fragments thereof disclosed herein, including amino acid deletions, additions, and substitutions, particularly conservative substitutions. The presently disclosed subject matter also encompasses modifications to increase in vivo half-life and decrease degradation in vivo. Substitutions, additions, and deletions can include, for example, 1, 2, 3, 4, 5, 6, 7, 8, 9, 10, 11, 12, 13, 14, 15, 16, 17, 18, 19, 20, 21, 22, 23, 24, and 25 changes as long as the activity disclosed herein remains substantially the same.

The presently disclosed subject matter includes an isolated nucleic acid comprising a nucleic acid sequence encoding a polypeptide of the presently disclosed subject matter, or a fragment or homolog thereof. In some embodiments, the nucleic acid sequence encodes a peptide comprising a polypeptide sequence of the presently disclosed subject matter, or a biologically active fragment of homolog thereof.

In some embodiments, a homolog of a polypeptide (full length or fragment) of the presently disclosed subject matter is one with one or more amino acid substitutions, deletions, or additions, and with the sequence identities described herein. In some embodiments, the substitution, deletion, or addition is conservative.

In some embodiments, the subject is a mammal. In some embodiments, the mammal is a human.

The presently disclosed subject matter encompasses the use of purified isolated, recombinant, and synthetic polypeptides. The presently disclosed subject matter also provides in some embodiments recombinant nucleic acids and substantially homologous nucleic acid sequences thereto. In some embodiments, the peptide or nucleic acid is present in the pharmacologically acceptable carrier. In some embodiments, the presently disclosed polypeptides, fragments, and homologs thereof can comprise a tag sequence, linker sequence, spacer sequence and/or other additional sequence that can be used in to facilitate expression, detection, stability, purification, isolation, or other desired feature or aspect. Multiple copies of such sequences can be employed. Such sequences can be added to the N-terminus, the C-terminus, or both of a polypeptide, fragment, or homolog thereof of the presently disclosed subject matter.

One of ordinary skill in the art will appreciate that based on the sequences of the components of the polypeptides disclosed herein they can be modified independently of one another with conservative amino acid changes, including, insertions, deletions, and substitutions.

In addition to the delivery vector described herein, based on the teachings herein, other viral vectors can be used as well. Indeed, constructs encoding proteins, polypeptides, or peptide fragments thereof of the presently disclosed subject matter may be generated using methods that are well known in the art. The presently disclosed vectors also include and/or further comprise non-plasmid and non-viral compounds which facilitate transfer or delivery of nucleic acid to cells, such as, for example, polylysine compounds, liposomes, and the like. By way of example and not limitation, therapeutic polypeptide-encoding constructs can be used for treatment methods in accordance with the presently disclosed subject matter. Exemplary methods are described in U.S. Pat. Nos. 10,105,451; 10,336,804; U. S. Patent Application Publication No. 2019/0000991; and U. S. Patent Application Publication No. 2019/0008909, the entire contents of which are herein incorporated by reference.

In accordance with the presently disclosed subject matter, a convenient method of introduction will be through the use of a recombinant vector that incorporates the desired gene, together with its associated control sequences. The preparation of recombinant vectors is well known to those of skill in the art and described in many references, such as, for example, Green et al., 2014, incorporated herein in its entirety. Additional descripton regarding the production of vectors, including promoters, sequences, and configurations can be found in the EXAMPLES and Figures.

It is understood that the nucleic acid coding sequences to be expressed are positioned in a vector adjacent to and under the control of a promoter, including but not limited to constitutive, inducible and/or tissue specific promoters. It is understood in the art that to bring a coding sequence under the control of such a promoter, one generally positions the 5′ end of the transcription initiation site of the transcriptional reading frame of the gene product to be expressed between about 1 and about 50 nucleotides “downstream” of (i. e; 3′ of) the chosen promoter.

Thus, a promoter is a region of a DNA molecule typically within about 100 nucleotide pairs upstream of (i. e., 5′ to) the point at which transcription begins (i. e., a transcription start site). That region typically contains several types of DNA sequence elements that are located in similar relative positions in different genes. Another type of discrete transcription regulatory sequence element is an enhancer. An enhancer imposes specificity of time, location and expression level on a particular coding region or gene. A major function of an enhancer is to increase the level of transcription of a coding sequence in a cell that contains one or more transcription factors that bind to that enhancer. An enhancer can function when located at variable distances from transcription start sites so long as a promoter is present. Particular representative examples of promoters and enhancers are set forth in the EXAMPLES.

As used herein, the phrase “enhancer-promoter” means a composite unit that contains both enhancer and promoter elements. An enhancer-promoter is operatively linked to a coding sequence that encodes at least one gene product. As used herein, the phrase “operatively linked” means that an enhancer-promoter is connected to a coding sequence in such a way that the transcription of that coding sequence is controlled and regulated by that enhancer-promoter. Approaches for operatively linking an enhancer-promoter to a coding sequence are well known in the art; the precise orientation and location relative to a coding sequence of interest is dependent, inter alia, upon the specific nature of the enhancer-promoter. An enhancer-promoter used in a vector construct of the presently disclosed subject matter can be any enhancer-promoter that drives expression in a cell to be transfected. By employing an enhancer-promoter with well-known properties, the level and pattern of gene product expression can be optimized.

For introduction of, for example, a therapeutic polypeptide, a vector construct that will deliver the gene to the affected cells is desired. Viral vectors can be used. Exemplary vectors include adenovirus vectors, retroviral vectors, vaccinia virus vectors, adeno-associated virus vectors, and lentivirus vectors, which have been successfully used to deliver desired sequences to cells and tend to have a high infection efficiency. Examples of non-viral vectors include, but are not limited to, plasmids, liposomes, polyamine derivatives of DNA and the like. In some embodiments, transient expression of a therapeutic polypeptide is desired. In such embodiments, non-viral vectors can be employed. Suitable vector-therapeutic polypeptide constructs are adapted for administration as pharmaceutical compositions, as described herein below. Viral promoters can also be of use in vectors of the presently disclosed subject matter, and are known in the art.

Upon a review of the instant disclosure, a therapeutically effective amount of a gene of interest is well within the reach of the skilled person. By way example with regard to dosing of adenoviral vectors, a representative dosage corresponds to at least 1×10¹² capsids/kg of body weight, at least 5×10¹² capsids/kg of body weight, or at least 1×10¹³ capsids/kg of body weight. AAV Quantification of AAV capsid particle titers is easily determined and is well known in the art (e. g., Kohlbrenner et al., 2012; Grimm et al; 1999.

Vectors in accordance with the presently disclosed subject matter can also comprise additional coding sequences, as might be envisioned for certain uses upon a review of the presently disclosed subject matter. For example, in some embodiments, vectors of the presently disclosed subject matter can comprise a nucleic acid sequence encoding a detectable marker. Particular representative examples of detectable markers are set forth in the EXAMPLES.

### III.C. Representative Treatment Methods and Preparation Therefor

In some embodiments, methods for treating a disease or disorder in a subject in need thereof are provided. In some embodiments, the disease or disorder is characterized by having kinase activity wherein a peptide domain that is stabilized when phosphorylated by the kinase activity, such as but not limited to extracellular regulated kinase (ERK) activity or a kinase activity associated with a stress response, such as p38 MAP kinase activity and/or INK activity. In some embodiments, the kinase activity is elevated. Cancer is an example of a disease or disorder. By way of particular example and not limitation, a representative, non-limiting cancer is any cancer where ERK, p38 MAP kinase, and/or JNK activity is relevant or a cancer undergoing chemotherapy or other therapy. GBM is a more particular but again non-limiting example of a cancer. In some embodiments, the method comprises administering to the subject a vector comprising a first nucleic acid sequence encoding a promoter operably linked to each of a second nucleic acid sequence encoding a therapeutic polypeptide, optionally a third nucleic acid sequence encoding a nuclear localization sequence (NLS), and a fourth nucleic acid sequence encoding a peptide domain that is stabilized when phosphorylated by kinase activity, such as but not limited to extracellular regulated kinase (ERK) activity, p38 MAP kinase activity, and/or JNK activity. In some embodiments, the method comprises administering to the subject a prodrug that is converted by the therapeutic polypeptide to an active agent, e. g. a toxic product.

Thus, the order of encoded peptides is changeable and in some embodiments not all peptides are included, such as in the fusion protein. For example in some embodiments the NLS is not included, such as in a fusion protein embodiment.

By the term “therapeutic polypeptide” it is meant a polypeptide that can have any sort of therapeutic effect in a cell and/or of interest for selective targeting through a peptide domain that is stabilized when phosphorylated by a kinase in a target issue. For example, this strategy, in some embodiments an Erk-regulated strategy, a p38 MAP kinase-regulated strategy, and/or a JNK-regulated strategy can be employed for cancer-selective expression of a number of therapeutic polypeptides, including but not limited to the following: (1) a suicide gene approach with activation of a prodrug; (2) expression of a secreted toxic protein, such as TRAIL; and (3) expression of a secreted immune cytokine or immune danger signal.

Thus, in some embodiments, the presently disclosed subject matter provides a strategy that yields kinase-based (e. g., Erk-based, p38 MAP kinase-based, and/or JNK-based), cancer-selective expression of any protein for treatment of of a disease or disorder associated with kinase activity, such as ERK activity, p38 MAP kinase activity, and/or INK activity, such as cancer, such as a cancer undergoing chemotherapy or other therapy.

In further embodiments, approaches for delivery enhance the impact and applicability of this overall strategy. In some embodiments, the vector can comprise a cell-penetrating peptide, such as an Antennapedia cell-penetrating peptide, which can facilitate transport of peptides and whole proteins through the cell membrane. By way of particular but non-limiting example, an Antennapedia cell-penetrating peptide is fused to to the N-terminus of HSVtk-FIRE. In vitro testing of this fusion protein is performed in GBM cells to ensure that it is able to cross the cell membrane and trigger cell death with ganciclovir exposure. If so, the local CED administration of the fusion protein is tested in the models disclosed herein, with a single dose administered seven days after GIC infusion. Daily ganciclovir dosing begins the day of protein infusion. In addition, the use of focused ultrasound (FUS) is leveraged to promote convection-enhanced delivery (CED) of a construct in accordance with the presently disclosed subject matter, such as but not limited to HSVtk-FIRE, in viral vectors or liposomes.

In some embodiments, the vector comprises a first nucleic acid sequence encoding a promoter operably linked to a second nucleic acid sequence encoding a fusion protein comprising the therapeutic polypeptide, optionally the NLS, and the peptide domain that is stabilized when phosphorylated by kinase activity, such as but not limited to extracellular regulated kinase (ERK) activity or a kinase activity associated with a stress response, such as p38 MAP kinase activity and/or INK activity. In some embodiments, the nucleic acid sequence encoding the fusion protein comprises: (a) a nucleic acid sequence encoding the therapeutic polypeptide; (b) a nucleic acid sequence encoding the NLS; and (c) a nucleic acid sequence encoding the peptide domain that is stabilized when phosphorylated by kinase activity, such as but not limited to extracellular regulated kinase (ERK) activity or a kinase activity associated with a stress response, such as p38 MAP kinase activity and/or INK activity. In some embodiments, the nucleic acid sequences of (a), (b), and (c) are fused in frame such that the NLS is at an N-terminus of the fusion protein and the peptide domain that is stabilized when phosphorylated by kinase activity, such as but not limited to extracellular regulated kinase (ERK) activity or a kinase activity associated with a stress response, such as p38 MAP kinase activity and/or INK activity, is at a C-terminus of the fusion protein.

In some embodiments, the therapeutic polypeptide comprises a Herpes simplex virus thymidine kinase (HSVtk) polypeptide or a yeast cytosine deaminase polypeptide. The therapeutic polypeptide is also referred to herein as a suicide gene product or as being encoded by a suicide gene.

In some embodiments, the therapeutic polypeptide comprises a Herpes simplex virus thymidine kinase (HSVtk) polypeptide or a yeast cytosine deaminase polypeptide. In some embodiments, the HSVtk polypeptide comprises a polypeptide selected from the group consisting of a polypeptide having an amino acid sequence as set forth in SEQ ID NO:1, a homolog thereof, a fragment thereof, or a homolog of the fragment thereof. In some embodiments, the HSVtk polypeptide comprises a polypeptide selected from the group consisting of a HSVtk polypeptide a polypeptide having an amino acid sequence having 95% homology to SEQ ID NO:1, and a fragment thereof. In some embodiments, the HSVtk polypeptide comprises a polypeptide selected from the group consisting of a polypeptide having an amino acid sequence as set forth in SEQ ID NO:1, a fragment thereof, a polypeptide having an amino acid sequence having 95% homology to SEQ ID NO:1, and a fragment thereof. In some embodiments, the amino acid sequence of the polypeptide comprises least one modification selected from the group consisting of an amino acid deletion, an amino acid addition, an amino acid substitution, and combinations thereof.

In some embodiments, the nucleic acid sequence is selected from the group consisting of (a) a nucleic acid sequence encoding a polypeptide having an amino acid sequence as set forth in SEQ ID NO: 1, or a fragment or homolog thereof, (b) a nucleic acid sequence as set forth in SEQ ID NO: 2, or its complementary strands; (c) a homologous nucleic acid sequence to a nucleic acid sequence as set forth in SEQ ID NO: 2, and which encodes a HSVtk polypeptide; and (d) a nucleic acid sequence differing from an isolated nucleic acid molecule of (a), (b), or (c) above due to degeneracy of the genetic code, and which encodes a HSVtk polypeptide encoded by the isolated nucleic acid molecule of (a), (b), or (c) above.

In some embodiments, the nucleic acid sequence is selected from the group consisting of: (a) a nucleic acid sequence encoding a polypeptide comprising an amino acid sequence as set forth in SEQ ID NO: 1; a fragment thereof, a polypeptide having an amino acid sequence having 95% homology to SEQ ID NO: 1, and a fragment thereof, (b) a nucleic acid sequence as set forth in SEQ ID NO: 2, or its complementary strands; (c) a nucleic acid sequence having 95% homology to a nucleic acid sequence as set forth in SEQ ID NO: 2, and which encodes a HSVtk polypeptide; and (d) a nucleic acid sequence differing from an isolated nucleic acid molecule of (a), (b), or (c) above due to degeneracy of the genetic code, and which encodes a HSVtk polypeptide encoded by the isolated nucleic acid molecule of (a), (b), or (c) above.

In some embodiments, the yeast cytosine deaminase (yCD) polypeptide comprises a polypeptide selected from the group consisting of a polypeptide having an amino acid sequence as set forth in SEQ ID NO: 5, a homolog thereof, a fragment thereof, or a homolog of the fragment thereof. In some embodiments, the yCD polypeptide comprises a polypeptide selected from the group consisting of a polypeptide having an amino acid sequence as set forth in SEQ ID NO: 5, a fragment thereof, a polypeptide having an amino acid sequence having 95% homology to SEQ ID NO: 5, and a fragment thereof. In some embodiments, the amino acid sequence of the polypeptide comprises least one modification selected from the group consisting of an amino acid deletion, an amino acid addition, an amino acid substitution, and combinations thereof.

In some embodiments, the nucleic acid sequence is selected from the group consisting of (a) a nucleic acid sequence encoding a polypeptide having an amino acid sequence as set forth in SEQ ID NO: 5, or a fragment or homolog thereof, (b) a nucleic acid sequence as set forth in SEQ ID NO: 6, or its complementary strands; (c) a homologous nucleic acid sequence to a nucleic acid sequence as set forth in SEQ ID NO: 6, and which encodes a yCD polypeptide; and (d) a nucleic acid sequence differing from an isolated nucleic acid molecule of (a), (b), or (c) above due to degeneracy of the genetic code, and which encodes a yCD polypeptide encoded by the isolated nucleic acid molecule of (a), (b), or (c) above.

In some embodiments, the nucleic acid sequence is selected from the group consisting of: (a) a nucleic acid sequence encoding a polypeptide comprising an amino acid sequence as set forth in SEQ ID NO: 5; a fragment thereof, a polypeptide having an amino acid sequence having 95% homology to SEQ ID NO: 5, and a fragment thereof, (b) a nucleic acid sequence as set forth in SEQ ID NO: 6, or its complementary strands; (c) a nucleic acid sequence having 95% homology to a nucleic acid sequence as set forth in SEQ ID NO: 6, and which encodes a yCD polypeptide; and (d) a nucleic acid sequence differing from an isolated nucleic acid molecule of (a), (b), or (c) above due to degeneracy of the genetic code, and which encodes a yCD polypeptide encoded by the isolated nucleic acid molecule of (a), (b), or (c) above.

In some embodiments, the peptide domain that is stabilized when phosphorylated by kinase activity, such as but not limited to extracellular regulated kinase (ERK) activity or a kinase activity associated with a stress response, such as p38 MAP kinase activity, comprises a Fra1-based integrative reporter (FTRE) polypeptide. In some embodiments, the FTRE polypeptide comprises a polypeptide selected from the group consisting of a polypeptide having an amino acid sequence as set forth in SEQ ID NO:4, a fragment thereof, a homolog thereof, a fragment thereof, or a homolog of the fragment thereof. In some embodiments, the FIRE polypeptide comprises a polypeptide selected from the group consisting of a polypeptide having an amino acid sequence as set forth in SEQ ID NO:4, a fragment thereof, a polypeptide having an amino acid sequence having 95% homology to SEQ ID NO:4, and a fragment thereof. In some embodiments, the FIRE polypeptide comprises amino acids 163-271 (also referred to herein as a PEST domain) of SEQ ID NO:4, or fragment or homolog thereof. In some embodiments, the amino acid sequence of the polypeptide comprises least one modification selected from the group consisting of an amino acid deletion, an amino acid addition, an amino acid substitution, and combinations thereofas compared to any one of SEQ ID NOs: 1, 4, 5, 7-20, 22, 24, 26, 28, 30, 32, and 34.

In some embodiments, the nucleic acid sequence is selected from the group consisting of (a) a nucleic acid sequence encoding a polypeptide having an amino acid sequence as set forth in SEQ ID NO: 4, or a fragment or homolog thereof, (b) a nucleic acid sequence as set forth in SEQ ID NO: 3, or its complementary strands; (c) a homologous nucleic acid sequence to a nucleic acid sequence as set forth in SEQ ID NO: 3, and which encodes a FIRE polypeptide; and (d) a nucleic acid sequence differing from an isolated nucleic acid molecule of (a), (b), or (c) above due to degeneracy of the genetic code, and 15 which encodes a FRE polypeptide encoded by the isolated nucleic acid molecule of (a), (b), or (c) above.

In some embodiments, the nucleic acid sequence is selected from the group consisting of: (a) a nucleic acid sequence encoding a polypeptide comprising an amino acid sequence as set forth in SEQ ID NO: 4; a fragment thereof, a polypeptide having an amino acid sequence having 95% homology to SEQ ID NO: 4, and a fragment thereof, (b) a nucleic acid sequence as set forth in SEQ ID NO: 3, or its complementary strands; (c) a nucleic acid sequence having 95% homology to a nucleic acid sequence as set forth in SEQ ID NO: 3, and which encodes a FIRE polypeptide; and (d) a nucleic acid sequence differing from an isolated nucleic acid molecule of (a), (b), or (c) above due to degeneracy of the genetic code, and which encodes a FTRE polypeptide encoded by the isolated nucleic acid molecule of (a), (b), or (c) above.

FIGS. 15 through 20 present representative amino acid sequences and nucleic acid sequences employed with p38 MAP kinase embodiments as disclosed herein. Also provided are substantially identical amino acid sequences and substantially identical nucleic acid sequences to the amino acid sequences and nucleic acid sequences disclosed in FIGS. 15-20. Also provided are fragments of the amino acid sequences and nucleic acid sequences disclosed in FIGS. 15-20 and substantially identical amino acid sequences and substantially identical nucleic acid sequences to the fragments. Also provided are complementary strands of any of these nucleic acids sequences and a nucleic acid sequence differing from any of the nucleic acids due to degeneracy of the genetic code, and which encodes an amino acid sequence encoded by the isolated nucleic acid molecule.

Any suitable NLS as would be apparent to one of ordinary skill in the art upon in a review of the instant disclosure can be employed. In some embodiments, a nuclear localization sequence is MAPKKKRK (SEQ ID NO: 7), or a homolog or fragment thereof. In some embodiments, an NLS is PKKKRKV (SEQ ID NO: 8), or a homolog or fragment thereof. In some embodiments, an NLS is KRPAATKKAGQAKKKK (SEQ ID NO: 9), or a homolog or fragment thereof. In some embodiments, an NLS is PAAKRVKLD (SEQ ID NO: 10), or a homolog or fragment thereof (see Makkerh et al., 1996). In some embodiments, the amino acid sequence of the polypeptide comprises least one modification selected from the group consisting of an amino acid deletion, an amino acid addition, an amino acid substitution, and combinations thereof.

In some embodiments, the vector and/or the prodrug are administered in a pharmaceutically acceptable diluent or vehicle. In some embodiments, the prodrug is selected from the group consisting of ganciclovir, acyclovir, and 5-fluorocytosine.

In some embodiments, the prodrug is selected from the group consisting of ganciclovir, acyclovir, and 5-fluorocytosine. Ganciclovir is a useful chemical (prodrug) of the presently disclosed subject matter for use with HSVtk. It has the following structure and chemical name:

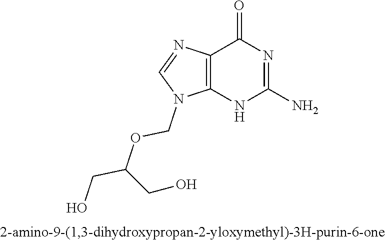 [SMILES: Nc1nc(=O)c2ncn(COC(CO)CO)c2[nH]1]

It comes in various doses/amounts, including 500 mg. In some embodiments, it can be used at about 0.1 to about 5,000 mg/kg/day, or about 1.0 to about 1,000 mg/kg/day, or about 2.0 to about 500/mg/kg/day, or about 0.1, 1, 2, 3, 4, 5, 6, 7, 8, 9, 10, 11, 12, 13, 14, 15 mg/kg. In some embodiments, oral or intravenous administration can be used, or can be used in combination or successively. In some embodiments, unit doses are used.

The vector (e. g., HSVtk-FIRE or a construct set forth in FIGS. 15-20) and the prodrug (e. g., GCV) can be administered simultaneously, or one can be administered after the other. Timing and the order of administration can be determined based on such things as the health, age, and sex of the subject.

In some embodiments, the disease or disorder is characterized by cells having up-regulated ERK due to high Ras or high RAF activity. In some embodiments, the disease or disorder is is characterized by cells undergoing a stress response and/or having upregulated p38 and/or INK activity. In some embodiments, the disease or disorder is cancer, such as a cancer being treated with chemotherapy or other therapy. In some embodiments, the cancer is glioblastoma.

In some embodiments, the method further comprises administering an additional therapeutic agent to the subject. In some embodiments, the additional therapeutic agent is an anti-cancer drug (e. g., temozolomide chemotherapy), radiation, or a combination thereof.

### III.D. Pharmaceutical Compositions and Administration

The presently disclosed subject matter is also directed to methods of administering the compositions of the presently disclosed subject matter to a subject.

Pharmaceutical compositions comprising the present vectors and/or prodrugs are administered to a subject in need thereof by any number of routes including, but not limited to, intratumoral, intracranial, topical, oral, intravenous, intramuscular, intra-arterial, intramedullary, intrathecal, intraventricular, transdermal, subcutaneous, intraperitoneal, intranasal, enteral, sublingual, or rectal approaches.

In accordance with one embodiment, a method of treating a subject in need of such treatment is provided. The method comprises administering a pharmaceutical composition comprising at least one composition of the presently disclosed subject matter to a subject in need thereof. Compositions provided by the methods of the presently disclosed subject matter can be administered with known compounds or other medications as well.

The pharmaceutical compositions useful for practicing the presently disclosed subject matter may be administered to deliver a dose of between 1 ng/kg/day and 100 mg/kg/day.

The presently disclosed subject matter encompasses the preparation and use of pharmaceutical compositions comprising a vector and/or prodrug useful for treatment of the diseases and disorders disclosed herein as an active ingredient. Such a pharmaceutical composition may comprise the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The compositions of the presently disclosed subject matter may comprise at least one vector and/or prodrug, one or more acceptable carriers, and optionally other vectors, prodrugs, polypeptides, and/or therapeutic agents.

For in vivo applications, the compositions of the presently disclosed subject matter may comprise a pharmaceutically acceptable salt. Suitable acids which are capable of forming such salts with for example a prodrug of the presently disclosed subject matter include inorganic acids such as hydrochloric acid, hydrobromic acid, perchloric acid, nitric acid, thiocyanic acid, sulfuric acid, phosphoric acid, and the like; and organic acids such as formic acid, acetic acid, propionic acid, glycolic acid, lactic acid, anthranilic acid, cinnamic acid, naphthalene sulfonic acid, sulfanilic acid and the like.

Pharmaceutically acceptable carriers include physiologically tolerable or acceptable diluents, excipients, solvents, or adjuvants. The compositions are in some embodiments sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions may also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e. g., tromethamine hydrochloride), additions (e. g., 0.01 to 10 mole percent) of chelants (such as, for example, DTPA or DTPA-bisamide) or calcium chelate complexes (as for example calcium DTPA or CaNaDTPA-bisamide), or, optionally, additions (e. g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) may be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

The compositions of the presently disclosed subject matter or pharmaceutical compositions comprising these compositions may be administered so that the compositions may have a physiological effect. Administration may occur enterally or parenterally; for example, intratumorally, intracranially, orally, rectally, intracisternally, intravaginally, intraperitoneally, locally (e. g., with powders, ointments or drops), or as a buccal or nasal spray or aerosol. Parenteral administration is an approach. Particular parenteral administration methods include intravascular administration (e. g., intravenous bolus injection, intravenous infusion, intra-arterial bolus injection, intra-arterial infusion and catheter instillation into the vasculature), peri- and intra-target cell and/or injection, subcutaneous injection or deposition including subcutaneous infusion (such as by osmotic pumps), intramuscular injection, and direct application to the target area, for example by a catheter or other placement device.

Where the administration of the composition is by injection or direct application, the injection or direct application may be in a single dose or in multiple doses. Where the administration of the compound is by infusion, the infusion may be a single sustained dose over a prolonged period of time or multiple infusions.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

A pharmaceutical composition of the presently disclosed subject matter may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Genaro, 1985, which is incorporated herein by reference.

Typically, dosages of the compound of the presently disclosed subject matter which may be administered to an animal, in some embodiments a human, range in amount from 1 g to about 100 g per kilogram of body weight of the animal. While the precise dosage administered will vary depending upon any number of factors, including but not limited to, the type of animal and type of disease state being treated, the age of the animal and the route of administration. In some embodiments, the dosage of the compound will vary from about 1 mg to about 10 g per kilogram of body weight of the animal. In some embodiments, the dosage will vary from about 10 mg to about 1 g per kilogram of body weight of the animal.

The compositions may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Suitable preparations include injectables, either as liquid solutions or suspensions, however, solid forms suitable for solution in, suspension in, liquid prior to injection, may also be prepared. The preparation may also be emulsified, or the compositions encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the preparation may also include minor amounts of auxiliary substances such as wetting or emulsifying agents, pH buffering agents, and/or adjuvants.

The presently disclosed subject matter also includes a kit comprising a composition of the presently disclosed subject matter and an instructional material which describes adventitially administering the composition to a cell or a tissue of a subject. In some embodiments, this kit comprises a (in some embodiments sterile) solvent suitable for dissolving or suspending a composition of the presently disclosed subject matter prior to administering the composition to the subject and/or a device suitable for administering the composition such as a syringe, injector, or the like or other device as would be apparent to one of ordinary skill in the art upon a review of the instant disclosure. See also FIG. 11A.

As used herein, an “instructional material” includes a publication, a recording, a diagram, or any other medium of expression which can be used to communicate the usefulness of the peptide of the presently disclosed subject matter in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a mammal. The instructional material of the kit of the presently disclosed subject matter may, for example, be affixed to a container which contains the multimeric peptide of the presently disclosed subject matter or be shipped together with a container which contains the peptide. Alternatively, the instructional material may be shipped separately from the container with the intention that the instructional material and the composition be used cooperatively by the recipient.

### III.E. Peptide Modification and Preparation

Peptide preparation is described herein above and in the EXAMPLES. It will be appreciated, of course, that the proteins or peptides of the presently disclosed subject matter may incorporate amino acid residues which are modified without affecting activity. For example, the termini may be derivatized to include blocking groups, i. e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i. e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Acid addition salts of the presently disclosed subject matter are also contemplated as functional equivalents. Thus, a peptide in accordance with the presently disclosed subject matter treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the presently disclosed subject matter.

The presently disclosed subject matter also provides for analogs of proteins. Analogs can differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function. To that end, 10 or more conservative amino acid changes typically have no effect on peptide function.

Modifications (which do not normally alter primary sequence) include in vivo, or in vitro chemical derivatization of polypeptides, e. g., acetylation, or carboxylation. Also included are modifications of glycosylation, e. g., those made by modifying the glycosylation patterns of a polypeptide during its synthesis and processing or in further processing steps; e. g., by exposing the polypeptide to enzymes which affect glycosylation, e. g., mammalian glycosylating or deglycosylating enzymes. Also embraced are sequences which have phosphorylated amino acid residues, e. g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e. g., D-amino acids or non-naturally occurring or non-standard synthetic amino acids. The peptides of the presently disclosed subject matter are not limited to products of any of the specific exemplary processes listed herein.

The presently disclosed subject matter includes the use of beta-alanine (also referred to as β-alanine, β-Ala, bA, and RA, having the structure:

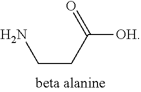

Sequences are provided herein which use the symbol “βA”, but in the Sequence Listing submitted herewith “βA” is provided as “Xaa” and reference in the text of the Sequence Listing indicates that Xaa is beta alanine.

It will be appreciated, of course, that the polypeptides, derivatives, or fragments thereof may incorporate amino acid residues which are modified without affecting activity. For example, the termini may be derivatized to include blocking groups, i. e. chemical substituents suitable to protect and/or stabilize the N- and C-termini from “undesirable degradation”, a term meant to encompass any type of enzymatic, chemical, or biochemical breakdown of the compound at its termini which is likely to affect the function of the compound, i. e. sequential degradation of the compound at a terminal end thereof.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the presently disclosed subject matter are also contemplated, for example, inverted peptides in which all amino acids are substituted with D-amino acid forms.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al., 1990.

As discussed, modifications or optimizations of peptide ligands of the presently disclosed subject matter are within the scope of the application. Modified or optimized peptides are included within the definition of peptide binding ligand. Specifically, a peptide sequence identified can be modified to optimize its potency, pharmacokinetic behavior, stability and/or other biological, physical, and chemical properties.

### III.F. Amino Acid Substitutions

In certain embodiments, the disclosed methods and compositions may involve preparing polypeptides with one or more substituted amino acid residues.

In various embodiments, the structural, physical and/or therapeutic characteristics of peptide sequences may be optimized by replacing one or more amino acid residues.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the presently disclosed subject matter are also contemplated, for example, inverted peptides in which all amino acids are substituted with D-amino acid forms.

The skilled artisan will be aware that, in general, amino acid substitutions in a peptide typically involve the replacement of an amino acid with another amino acid of relatively similar properties (i. e., conservative amino acid substitutions). The properties of the various amino acids and effect of amino acid substitution on protein structure and function have been the subject of extensive study and knowledge in the art.

For example, one can make the following isosteric and/or conservative amino acid changes in the parent polypeptide sequence with the expectation that the resulting polypeptides would have a similar or improved profile of the properties described above: Substitution of alkyl-substituted hydrophobic amino acids: including alanine, leucine, isoleucine, valine, norleucine, S-2-aminobutyric acid, S-cyclohexylalanine or other simple alpha-amino acids substituted by an aliphatic side chain from 1-10 carbons including branched, cyclic and straight chain alkyl, alkenyl or alkynyl substitutions.

Substitution of aromatic-substituted hydrophobic amino acids: including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo, or iodo) or alkoxy-substituted forms of the previous listed aromatic amino acids, illustrative examples of which are: 2-,3- or 4-aminophenylalanine, 2-,3- or 4-chlorophenylalanine, 2-,3- or 4-methylphenylalanine, 2-,3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2,3, or 4-biphenylalanine, 2′,-3′,- or 4′-methyl-2, 3 or 4-biphenylalanine, and 2- or 3-pyridylalanine.

Substitution of amino acids containing basic functions: including arginine, lysine, histidine, ornithine, 2,3-diaminopropionic acid, homoarginine, alkyl, alkenyl, or aryl-substituted (from C1-C10 branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of acidic amino acids: including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids.

Substitution of side chain amide residues: including asparagine, glutamine, and alkyl or aromatic substituted derivatives of asparagine or glutamine.

Substitution of hydroxyl containing amino acids: including serine, threonine, homoserine, 2,3-diaminopropionic acid, and alkyl or aromatic substituted derivatives of serine or threonine. It is also understood that the amino acids within each of the categories listed above can be substituted for another of the same group.

For example, the hydropathic index of amino acids may be considered (Kyte & Doolittle, 1982). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, amino acids having hydropathic indices that are in some embodiments within +/−2, in some embodiments within +/−1, and in some embodiments within +/−0.5 can be employed.

Amino acid substitution may also take into account the hydrophilicity of the amino acid residue (e. g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In some embodiments, replacement of amino acids with others of similar hydrophilicity is employed.

Other considerations include the size of the amino acid side chain. For example, an amino acid with a compact side chain, such as glycine or serine, would not ideally be replaced with an amino acid with a bulky side chain, e. g., tryptophan or tyrosine. The effect of various amino acid residues on protein secondary structure is also a consideration.

Through empirical study, the effect of different amino acid residues on the tendency of protein domains to adopt an alpha-helical, beta-sheet or reverse turn secondary structure has been determined and is known in the art (see e. g., Chou & Fasman, 1974; Chou & Fasman, 1978; Chou & Fasman, 1979).

Based on such considerations and extensive empirical study, tables of conservative amino acid substitutions have been constructed and are known in the art. For example: arginine and lysine; glutamate and aspartate; serine and threonine; glutamine and asparagine; and valine, leucine, and isoleucine. Alternatively: Ala (A) Leu, Ile, Val; Arg (R) Gln, Asn, Lys; Asn (N) His, Asp, Lys, Arg, Gln; Asp (D) Asn, Glu; Cys (C) Ala, Ser; Gln (Q) Glu, Asn; Glu (E) Gln, Asp; Gly (G) Ala; His (H) Asn, Gln, Lys, Arg; Ile (I) Val, Met, Ala, Phe, Leu; Leu (L) Val, Met, Ala, Phe, Ile; Lys (K) Gln, Asn, Arg; Met (M) Phe, Ile, Leu; Phe (F) Leu, Val, Ile, Ala, Tyr; Pro (P) Ala; Ser (S), Thr; Thr (T) Ser; Trp (W) Phe, Tyr; Tyr (Y) Trp, Phe, Thr, Ser; Val (V) Ile, Leu, Met, Phe, Ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. See e. g., PROWL Rockefeller University website. For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix.

In determining amino acid substitutions, one may also consider the existence of intermolecular or intramolecular bonds, such as formation of ionic bonds (salt bridges) between positively charged residues (e. g., His, Arg, Lys) and negatively charged residues (e. g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

### EXAMPLES

The following EXAMPLES are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Example 1

Many cancers involve aberrant signaling through the Ras/Raf/MEK/ERK pathway. While pharmacological inhibitors exist to target some of the nodes in this signaling cascade, cancer cells can leverage multiple opportunities to develop resistance to those inhibitors, most often in ways that lead to maintenance of ERK signaling. Because maintenance of ERK signaling in cancer cells can be a potent driver of cancer cell survival, there is a need for new and orthogonal mechanisms to target this signaling pathway. The presently disclosed subject matter provides a simple, yet effective, way to accomplish that goal by turning a cancer cell's ability to drive the ERK pathway against itself. As shown in a representative, non-limiting manner in this EXAMPLE, this approach involves the design of an ERK-stabilized suicide gene, which converts the prodrug ganciclovir (GCV) into a toxic product through the expression of the Herpes simplex virus thymidine kinase (HSVtk) protein. An aspect of this construct is the fusion of the HSVtk protein to a nuclear localization sequence (NLS) and a peptide domain (Fra1 domain) that is stabilized when phosphorylated by active ERK, which is preferentially shuttled to the cell nucleus (FIGS. 1A and 1i). This fusion protein is referred to as HSVtk-FIRE, where FIRE stands for Fra1-based integrative reporter. The FIRE acronym borrows from a paper in the scientific literature that describes the basic NLS/Fra1 fusion approach to generate an ERK-stabilized fluorescent fusion protein, which can be used as a live-cell reporter of ERK activity (Albeck et al., 2013). However, the presently disclosed subject matter provides for the first implementation of this approach to create an ERK-stabilized suicide gene. The fusion of HSVtk with the NLS and Fra1 domains creates an ERK-stabilized HSVtk protein, which provides for ERK-specific killing of cancer cells (see FIGS. 2A and 2B).

The fusion protein construct has been inserted into a retroviral expression vector backbone. This vector is transfected into packaging cell lines to produce retroviral particles that are injected locally into sites where ERK-dependent tumor cells are present. Because retroviral vectors preferentially transduce dividing cells, retroviral packaging can further afford some degree of cancer cell-specific transduction versus normal cell counterparts that may be less rapidly dividing. As desired, the construct can be inserted into another type of vector backbone.

### Example 2

This EXAMPLE relates to the presently disclosed suicide gene therapy approach implemented to selectively kill glioblastoma multiforme (or other human cancer) cells that display elevated Ras/ERK signaling, a hallmark of many cancers.

Approximately 14,000 new cases of glioblastoma multiforme (GBM) are diagnosed in the United States each year. Median survival time for these patients is a dismal 18 months due to GBM resistance to current modalities of chemoradiation and a general inability to resect surgically tumor cells that diffusely spread. It is estimated that as many as 90% of all GBM tumors display dysregulation of receptor-mediated signaling processes that drive Ras/ERK signaling. Thus, the vast majority of the GBM patient population can benefit from new approaches to target Ras/ERK signaling. However, this is also more broadly applicable to the large subset of human cancer driven by Ras.

GBM is the most common adult malignancy of the brain. GBM is characterized by diffuse invasion within the brain, and extremely rapid progression. Indeed, even with standard of care including surgery and radiation or chemotherapy, median survival is approximately 18 months (Stupp et al., 2005). Efforts to identify new and durable GBM therapies have been met with failure after failure. For example, within the last few years, there was initial excitement about the possibility that anti-angiogenic therapy could hold promise for GBM patients based on radiographic evidence showing some degree of impairment in tumor progression. However, as with all other approaches that have been attempted, patient survival was ultimately not prolonged with this approach. Thus, there is a need for creative new approaches for the treatment of GBM.

GBM is marked by a host of genetic lesions, but extensive efforts by The Cancer Genome Atlas project have distilled this disorder down to three main pathways. Of these, the largest and most complex is the receptor tyrosine kinase/Ras/PI3kinase pathways, with dysregulation of these in 88% of GBMs. Canonical Ras signaling, which can be simplified to the cascade of Ras→RAF→MEK→ERK, is at the heart of these pathways and is thus dys-regulated in the large majority of GBMs. Other brain tumors are also marked by Ras pathway disruptions; a number of brain tumor types are driven by lesions in BRAF. Ras remains a critical but frustratingly elusive target across oncology, and despite concerted efforts in recent years there is not yet an effective approach to inhibit Ras in cancer. This Example describes a novel approach to turn Ras activation against GBM and other cancers, bypassing the need to inhibit Ras and instead killing cancer cells with high Ras activity. It leverages a strategy for a protein reporter for extracellular regulated kinase (ERK) activity, a downstream mediator of Ras.

ERK 1 and 2 phosphorylate and stabilize a domain of the Fra-1 protein that otherwise enables degradation of the protein and fusing a nuclear localization signal (NLS) and this Fra-1 domain to luciferase yields a fluorescent protein that is degraded unless ERK phosphorylates the Fra-1 domain, thus providing a reporter for ERK activity. This approach has been adapted by conjugating the ERK-sensitive Fra-1 domain to the suicide gene herpes simplex virus thymidine kinase (HSVtk), resulting in a fusion protein that drives ERK-regulated cell death rather than fluorescence. Data indicate that expression of this construct, abbreviated HSVtk-FIRE (HSVtk-Fra1-based integrative reporter), plus the activating drug ganciclovir drives GBM cell death to a degree that correlates with ERK activity. Without wishing to be bound by any particular theory, HSVtk-FIRE can be delivered as a novel approach to selectively kill GBM cells with high Ras activity.

In this EXAMPLE, the abilities of the HSVtk-FIRE system to selectively kill specific GBM tumor cell types and to cooperate with approved or investigational therapeutics are demonstrated. Data supports the ability of the HSVtk-FIRE system to selectively kill specific types of GBM cells in a real tumor context, and as well as the ability of the system to be tuned to improve selectivity. In a real clinical application, the suicide gene is transduced to both tumor and non-tumor cells. Tumor cells themselves represent a heterogeneous mixture of cells with variable degrees of tumor-initiating and chemoresistance potential. Based on these and related points, this Example directly tests the ability of the suicide gene to selectively kill GBM cells when cultured alongside immortalized human astrocytes or fibroblasts. This Example also tests the efficacy of the suicide gene in glioblastoma-initiating cells (GICs), which are widely viewed as being able (in very small numbers) to drive the formation of fully differentiated GBM tumors and which are also widely viewed as being particularly resistant to chemotherapy. This Example also evaluates the suicide gene cooperation with approved or investigational systemic therapies.

Determination of whether HSVtk-FIRE delivery is an effective therapy in mouse models of GBM. To be effective against GBM, our fusion protein-based strategy employs relatively efficient delivery (though the HSVtk system yields bystander killing of cancer cells, enabling strong activity with much less than 100% delivery to target cancer cells). Approaches are compared for delivery of HSVtk-FIRE to treat GBM in orthotopic mouse models with GICs. Local injection of a retrovirus bearing HSVtk-FIRE into GBM with convection-enhanced delivery (CED). Combinatorial effects of local viral delivery of HSVtk-FIRE alongside standard-of-care radiation and temozolomide are also tested.

### Background for Example 2

Need for novel approaches to GBM therapy: GBM is universally lethal, with a median survival after diagnosis of 18 months (Stupp et al., 2005). Application of new treatments with breakthrough efficacy in other cancers has thus far been disappointing. Much of the resistance stems from GBM genetic heterogeneity and adaptability, and there have been extensive efforts to characterize common threads in GBM genetics and signaling that can be targeted to yield effective therapy. The Cancer Genome Atlas profiled several hundred GBMs and divided genetic lesions into three major categories: RTK/Ras/PI3K signaling pathways (88% of GBMs), the p53 pathway (87% of GBMs), and the cell cycle pathway (78% of GBMs; Cancer Genome Atlas Research Network, 2008). It is clear from these studies that Ras signaling is upregulated in the large majority of GBMs, but this has not yielded therapeutic gains.

Gap in Ras-directed therapy for GBM and other cancers: Ras is a GTPase and acts through regulation of downstream kinases via a Ras4RAF4MEK4ERK pathway. Not only is Ras dys-regulated in most GBMs, but the pathway is also a driver in many other brain tumors in children and adults through fusions and mutations in the BRAF gene. While RAF and MEK inhibitors have existed for some time, their benefit in GBM and some other Ras-driven cancers has been limited. Ras inhibitors have been far more difficult to develop, as it is not a kinase. A major initiative is now underway to identify approaches to target Ras in cancer, but no clear answers have emerged to date. This Example provides a new approach for therapy of Ras-driven cancers, through a protein construct with selective killing of cells with high ERK activity.

Leveraging Erk/Fra-1 regulation: Fra-1 is a target of ERK, and includes a domain that triggers protein degradation unless phosphorylated by ERK. A previous report showed that this Fra-1 domain could be fused to a fluorescent reporter to yield a fusion protein with increased activity correlating tightly with increased ERK activity (see Albeck et al., 2013). Importantly, a nuclear localization signal is added to the fusion protein, as ERK activity is primarily in the nucleus. An aspect of the presently disclosed subject matter adapts this into a therapy, fusing the Fra-1 domain to Herpes simplex virus thymidine kinase (HSVtk) to yield an ERK-regulated suicide gene, in essence, creating a construct that selectively kills GBM and other cancer cells with high Ras activity.

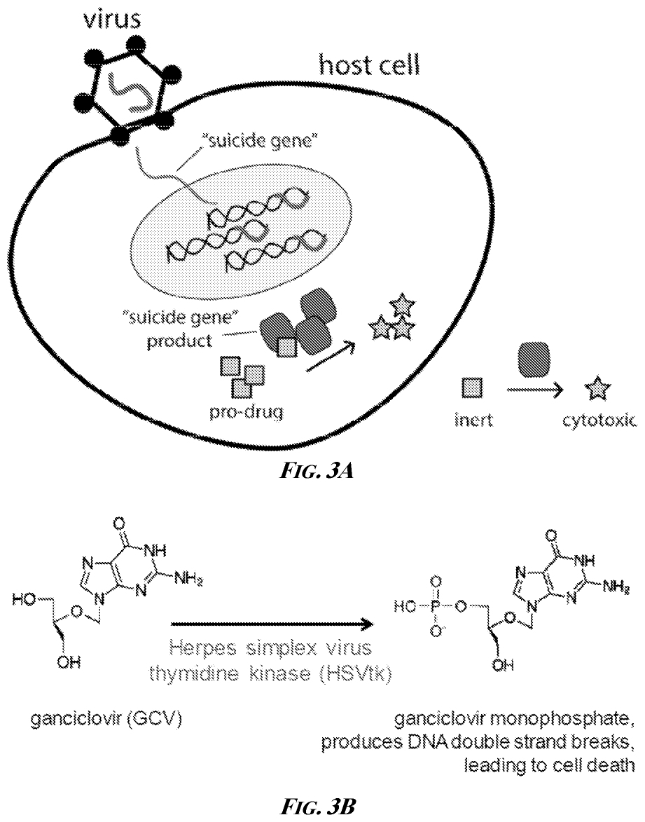

HSVtk/ganciclovir as a suicide gene approach to cancer: HSVtk expression in dividing cells results in cell death when combined with exposure to the herpes medications acyclovir or ganciclovir, through phosphorylation of these nucleoside analogs into aberrant nucleotides that are incorporated into DNA (FIG. 3; see also Fillat et al., 2003). There may also be mechanisms by which this system has some toxicity to non-dividing cells as well. The system is well known to result in “bystander killing”, with phosphorylated acyclovir/ganciclovir from HSVtk-expressing cells reaching neighboring cancer cells not expressing HSVtk and killing them as well. HSVtk is the most widely used “suicide gene” in preclinical cancer strategies, with numerous reports of its use to treat GBM and other cancers. In nearly all cases, these strategies incorporate some approach to direct the HSVtk toxicity selectively to cancer cells, but such selective localization has often proven challenging (Karjoo et al., 2016). The presently disclosed subject matter is belived to provide the first example of Ras-regulated, HSVtk-mediated cancer cell killing.

This strategy is the first to apply the ERK/Fra-1 interaction for therapeutic purposes. It represents a novel approach to selectively kill GBM and other cancer cells with up-regulated ERK due to high Ras or high RAF activity. A range of approaches is used for delivery of this unique targeted fusion protein.

### Preliminary Studies for Example 2

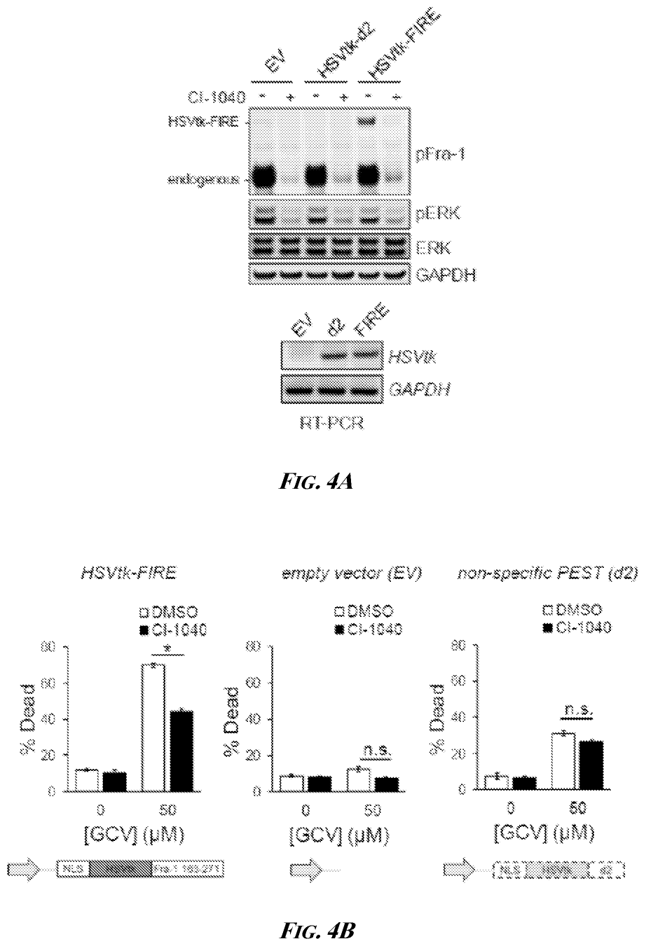

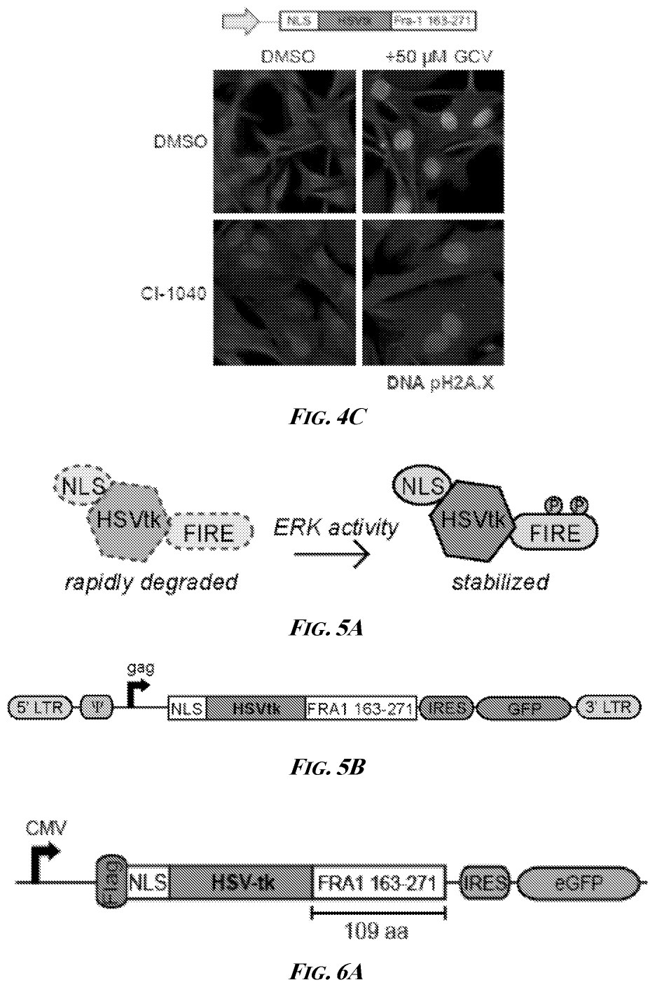

Basic design of the HSVtk-FIRE ERK activity-dependent suicide gene. We chose to base the design of our suicide gene construct on a recently described live-cell fluorescent reporter for ERK activity (Albeck et al., 2013). The basic design of that reporter involved the fusion of mVenus with a nuclear localization sequence on one end and a segment of the Fra-1 transcription factor (a substrate of ERK) on the other (FIG. 4A). The nuclear localization sequence directs the reporter to the nucleus, where active ERK is preferentially shuttled from the cytoplasm. The Fra-1 domain, when phosphorylated by ERK, is stabilized (i. e., its otherwise relatively rapid degradation is slowed). In this way, active ERK stabilizes the expression of mVenus (Albeck et al., 2013). The design of the presently disclosed ERK activity-dependent suicide gene replaced mVenus with the gene encoding HSVtk (FIG. 4B). This construct was inserted into a retroviral expression vector backbone.

Preliminary demonstration of the efficacy of the HSVtk-FIRE suicide gene. To demonstrate the basic efficacy of the ERK activity dependent HSVtk suicide gene, the well-known GBM cell line U87MG was transduced with the retroviral expression vector. In this case, a variant of U87MG cells engineered to have stable expression of the epidermal growth factor receptor (EGFR) mutant EGFRvIII because EGFRvIII (which is expressed in ˜25% of all GBM tumors) drives especially aggressive disease was employed. Moreover, EGFRvIII displays constitutive phosphorylation and has therefore been described as a potential driver of Ras/ERK signaling. After selection of stable cell lines, expression of the suicide gene construct was validated, and expression of a higher molecular weight version of Fra-1 was observed, which represented the HSVtk-FIRE fusion (FIG. 5A). As expected, expression of the fusion protein was substantially reduced in response to the MEK inhibitor CI-1040, which reduces downstream ERK activity. When GCV was added to these cells, U87MG cells exhibited substantial cell death as measured by cell permeability to ToPro3 using flow cytometry (FIG. 5B). Interestingly, when MEK/ERK signaling was inhibited using CI-1040, GCV-dependent cell death was greatly diminished. This experiment demonstrates an aspect of the HSVtk-FIRE system. Whereas MEK/ERK inhibition would ordinarily be anticipated to drive increased cell death in GBM and many other cancer cell lines, it does just the opposite here due to the expression of an ERK activity-dependent suicide gene.

Deeper validation. To further validate system specificity, U87MG cells were engineered expressing a version of the reporter wherein the Fra1 PEST domain is not regulated by ERK activity (termed the “d2” version of the construct), as well as an empty vector (EV) control (FIG. 6A). Cells expressing the EV were not responsive to GCV (FIG. 6B). Cells expressing the d2 version of the fusion protein did not display Fra1 phosphorylation (FIG. 6A) and were not responsive to MEK/ERK inhibition (FIG. 6B). The relatively modest (non-ERK-regulated) degree of GCV-mediated cell death observed in the d2-expressing cells is due to the active HSVtk expressed in those cells, which cannot be stabilized by ERK activity. Where observed, cell death in these cells was accompanied by clear signaling-based signs of DNA damage response (FIG. 6C).

Demonstrating the ability of the HSVtk-FIRE system to selectively kill specific GBM tumor cell types and to test its ability to cooperate with approved or investigational therapeutics. The selectivity of HSVtk-FIRE in a mixed cell population is assessed, as is the selective killing of GBM when cultured in various patterns with immortalized human astrocytes or human fibroblasts. The ERK regulation of killing by HSVtk-FIRE is also tested. GBM-initiating cells (GICs) have proven particularly resistant to most therapies, and in some cases this may be due to increased DNA repair capacity; therefore, in a large panel of GIC lines it is tested whether they are equally sensitive to killing by HSVtk-FIRE plus ganciclovir, and whether this killing correlates with any characteristics of the lines. Also tested in vitro is the combinatorial potential of HSVtk-FIRE alongside radiation, temozolomide, and PARP inhibition.

Testing whether HSVtk-FIRE selectively kills GBM cells and those with high ERK activity: Established GBM lines are lentivirally tagged with GFP and co-cultured with immortalized human astrocytes. Retrovirus bearing HSVtk-FIRE are added to wells and daily ganciclovir added beginning the next day. TUNEL assay is performed to test for apoptosis and correlate TUNEL staining with fluorescently marked GBM cells. As an additional approach, flow cytometry methods are employed to assess cell death, as in the preliminary data above. Also, whether transfection of immortalized astrocytes with vectors with an oncogenic Ras mutant or constitutively active ERK increase killing by HSVtk-FIRE in the above assays is evaluated.

Determining if HSVtk-FIRE is equally active in GIC lines and assess potential biomarkers: The efficacy of HSVtk-FIRE+ganciclovir is tested in a panel of ten well-characterized GIC lines. If there is variability in sensitivity across the lines, it is sought to correlate susceptibility with GBM subtype, EGFR status, p53 status, and radiation sensitivity. The same methods mentioned above are used.

Investigating potential synergies in combining HSVtk-FIRE with other therapies: HSVtk and ganciclovir lead to aberrant nucleotide incorporation and thus DNA damage. 10 Upfront therapy for GBM includes radiation and temozolomide, both DNA-damaging agents. Without wishing to be bound by any particular theory, it is hypothesized herein that combining HSVtk-FIRE with either of these leads to a two-pronged attack on tumor DNA and potential synergy. In a similar vein, PARP inhibitors can escalate DNA damage from other agents. The combination of HSVtk-FIRE/ganciclovir with radiation, temozolomide, or veliparib for in vitro toxicity is also tested in GIC lines. Data are analyzed to identify combinations of therapy that may display synergy, rather than purely additive effects. Those approaches are prioritized for testing in vivo.

Expected results/Significance: It is expected that this Example provides additional evidence of ERK-dependent selective killing of GBM and Ras/ERK-high cells by HSVtk-FIRE. This Example is also expected to show HSVtk-FIRE toxicity to GICs. It is difficult to predict whether toxicity will correlate with GBM subtype, but it is anticipated that p53 status and radiation sensitivity may be relevant as they are relevant to DNA damage repair. It is also expected that this Example shows synergistic activity with at least one or two of these other agents, and this then informs how best to apply HSVtk-FIRE alongside other agents in trials for patients with GBM.

Alternative approaches: Bystander killing by HSVtk-FIRE+ganciclovir might lead to killing of admixed immortalized astrocytes, as they are still dividing (albeit at a slower rate). If so, relative toxicity with the GBM cells and astrocytes is tested in separate cultures or separated by a membrane in transwells. While the lines testing above might not provide statistically robust correlations, an idea of markers for relative toxicity is expected and then the number of lines is expected to better assess this. If synergistic combinations with HSVtk-FIRE are not shown, additional testing of other agents such as lomustine and carboplatin is carried out.

Determining whether HSVtk-FTRE delivery is an effective therapy in mouse models of GBM. While the HSVtk-FIRE strategy represents a new approach to therapy of Ras-driven cancers, its effectiveness involves adequate delivery to cancer cells and its cooperation with existing therapies. The bystander killing of HSVtk should obviate the need to reach 100% of the GBM cells, but efficient delivery plays a role in therapy. An HSVtk-FIRE construct is incorporated into a retroviral vector in the preliminary studies described herein, and local convection-enhanced delivery of this retrovirus with systemic ganciclovir administration is tested for its efficacy in orthotopic mouse GIC models. Given the DNA-damaging activity of HSVtk/ganciclovir and the upfront GBM therapies radiation and temozolomide, this combination is also tested.

Testing whether local retroviral delivery of HSVtk-FIRE into GBM with CED is effective against GBM. Groups of ten SCID mice are stereotactically injected in the right cerebrum with one of two GIC lines. Then, seven days later, when tumors are established, the mice receive CED of 10⁷ plaque-forming unites (pfu) of retrovirus/HSVtk-FIRE or control retrovirus. Three days after this, the mice begin treatment with ganciclovir 50 mg/kg/day. Notably, the HSVtk/ganciclovir system has been shown effective in orthotopic glioma mouse models in the past (see e.,g., Amano et al., 2011). Brain MRI is performed four weeks after GIC injections to assess tumor size, comparing across groups. Mice are also followed for survival.

Determining the efficacy of HSVtk-FIRE in combination with radiation and temozolomide in treating established GIC xenografts. After initial diagnosis, patients with GBM are almost universally treated with radiation and the chemotherapy drug temozolomide. It is evaluated whether the addition of HSVtk-FIRE to this regimen markedly increases therapeutic efficacy. One of two GIC lines are stereotactically injected into the brains of SCID mice. Then, seven days later, when tumors are established, mice begin treatment with radiation/temozolomide/HSVtk-FIRE. The eight groups include [sham radiation+tem vehicle+HSVtk-FIRE sham injection], [radiation+tem vehicle+HSVtk-FIRE sham injection], [sham radiation+temozolomide+HSVtk-FIRE sham injection], [sham radiation+tem vehicle+HSVtk-FTRE], [radiation+temozolomide+HSVtk-FIRE sham injection], [radiation+tem vehicle+HSVtk-FIRE], [sham radiation+temozolomide +HSVtk-FTRE], and [radiation+temozolomide+HSVtk-FIRE]. 5 Gray radiation and temozolomide 50 mg/kg daily (on days 1-5 of 28-day cycles) are employed. The doses of both have been chosen to yield minor efficacy individually. HSVtk-FIRE treatment is done as above. Brain MRI are performed after three weeks of treatment to gauge tumor size, and mouse survival times are followed. Tumor size/mouse survival are compared across the eight groups.

Expected results/Significance: It is expected that the local retroviral delivery of HSVtk-FIRE decreases GBM size and prolong mouse survival. Furthermore, synergistic anti-GBM effects are expected in combining this treatment with radiation and temozolomide. The results of these studies establish the feasibility of HSVtk-FIRE as a Ras pathway-targeted strategy.

Alternative Approaches: If no efficacy is evident in the initial experiments with retroviral delivery of HSVtk-FIRE, retesting is done with increased doses of the retrovirus and administering second and third retrovirus infusions is considered. If delivery is inadequate with this local CED/retrovirus approach, adenovirus or other viral vectors, as well as liposomes or other nanoparticles with or without focused ultrasound enhancement, are employed. Obstacles are not with radiation and temozolomide treatment in the models described herein.

### Example 3

An alternative embodiment of the presently disclosed FIRE suicide gene system was prepared. Referring to FIGS. 5A and 5B, a Herpes simplex virus thymidine kinase sequence (HSVtk) was used with a nuclear localization sequence (NLS), and an FRA-1 integrated reporter element (FRA-1 PEST domain) (FIRE). The construct is used with ganciclovir (GCV), a prodrug converted to a toxic product by HSVtk. The expression vector also includes a green fluorescence protein (GFP) sequence. Phosphorylation of the FIRE domain by ERK stabilizes HSVtk-FIRE fusion protein expression, creating an alternative ERK-selective suicide gene product.

### Example 4

In order to validate appropriate subcellular localization of the NLS-HSVtk-FIRE construct, HSVtk expression constructs which contain an N-terminal Flag-tag were prepared. These constructs demonstrate that the NLS and FIRE elements of the construct are causing the anticipated effects on localization. See FIGS. 6A and 6B.

### Example 5

It was shown that HSVtk-FIRE promotes ERK-dependent cell killing in glioblastoma cells. Referring to FIGS. 7A and 7B, decreased ERK activation, due to treatment with the MEK inhibitor CI-1040, causes reduced cell death in response to GCV in glioblastoma cells expressing HSVtk-FIRE, compared to an ERK-independent PEST domain (d2) counterpart. Similar results observed in G88 glioma stem cells treated with the MEK inhibitor trametinib.

### Example 6

It was shown that HSVtk-FIRE promotes ERK-dependent cell killing through KRAS mutant-expressing pancreas cells. Referring to FIG. 8, normal human pancreatic ductal epithelial cells (HPDE), transduced with oncogenic KRAS (KRAS^{G12V}), show increased levels ERK and NLS-HSVtk-FIRE phosphorylation when compared to matched control cells transduced with an empty vector (EV). The HPDE cells expressing KRAS^{G12V} have greater levels of DNA damage (pH2A. X) in response to GCV.

### Example 7

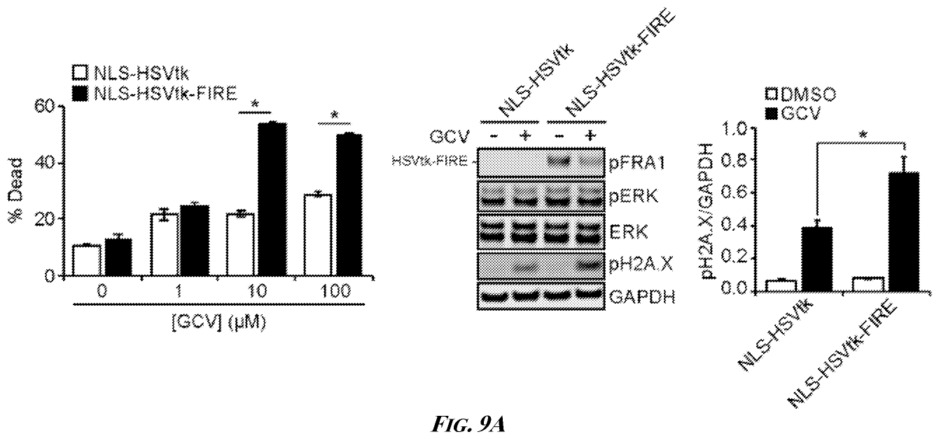

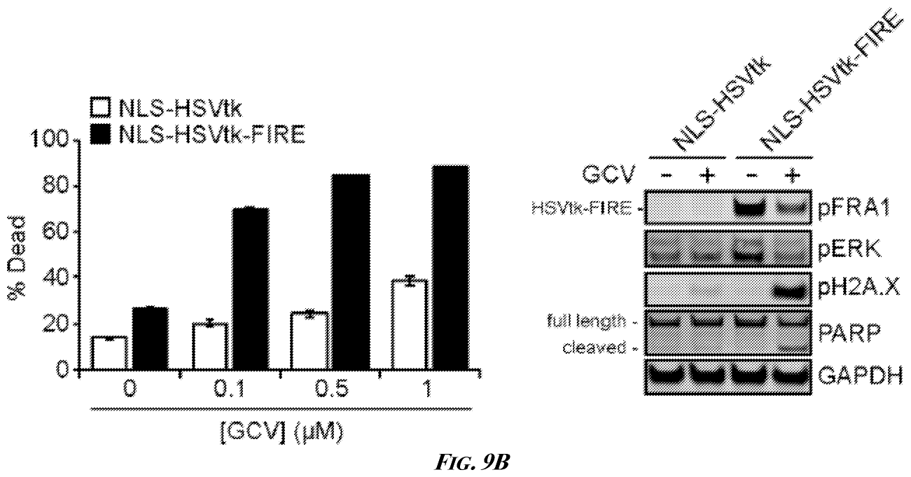

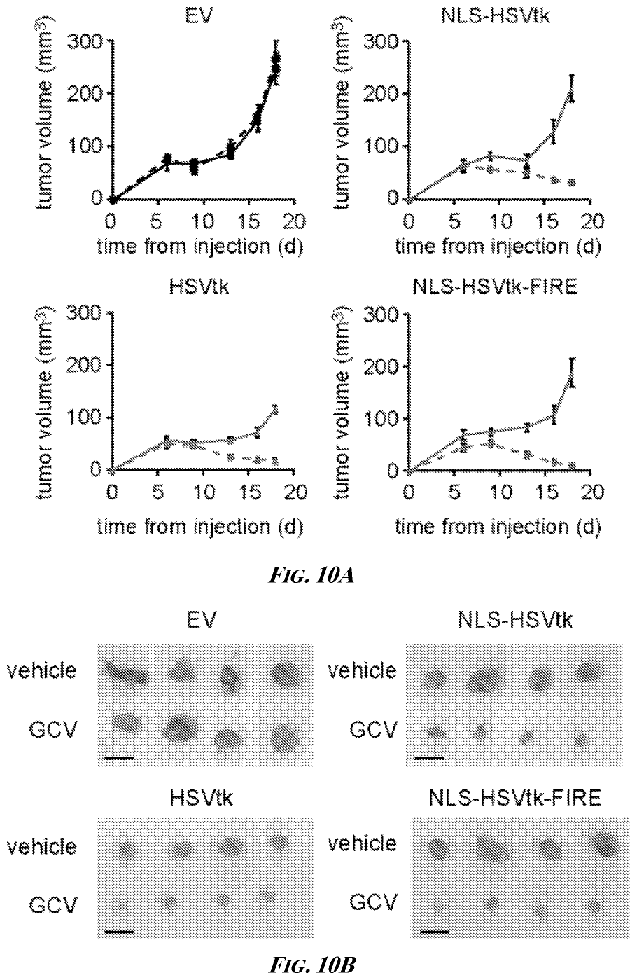

A comparison of HSVtk-FIRE against HSVtk lacking a PEST domain was made. Referring to FIGS. 9A and 9B, U87MG cells equally transduced with nuclear-localized HSVtk respond greater to GCV when the FIRE PEST domain is included (U87MG+EGFRvIII, FIG. 9A). Similar observations were made with G88 cells (FIG. 9B).

### Example 8

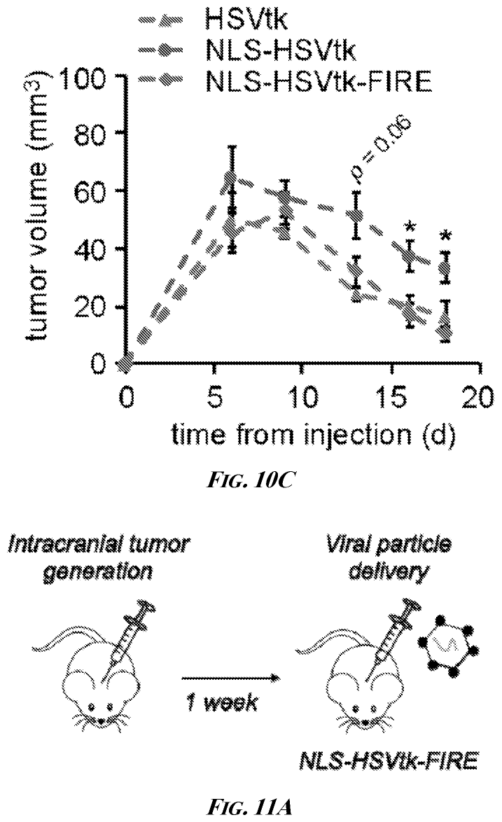

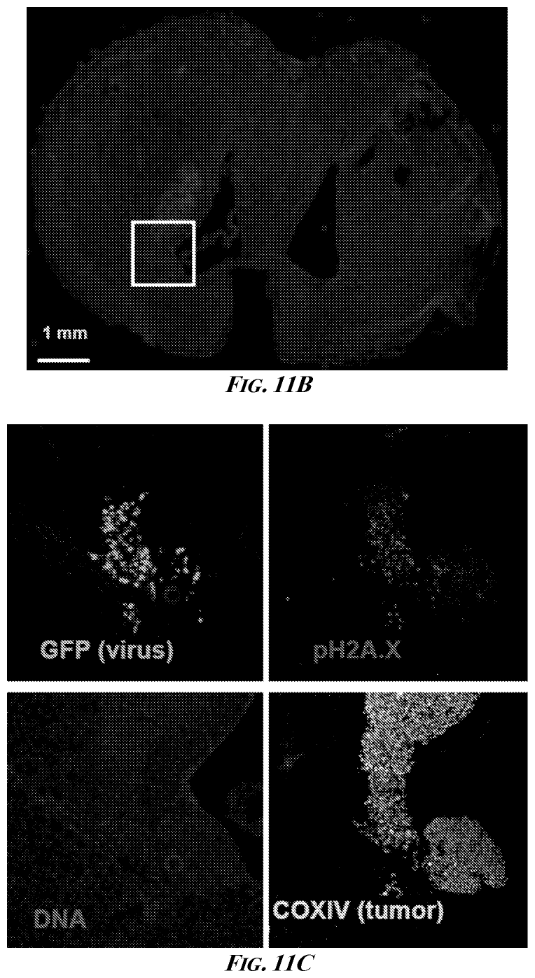

Subcutaneous xenograft experiments were performed, demonstrating an advantage of HSVtk fusion protein. Referring to FIGS. 10A-10C, U87MG cells equally transduced with nuclear-localized HSVtk respond greater to GCV, in a subcutaneous mouse model, when the FIRE PEST domain is included. In this Example it was found that NLS-HSVtk-FIRE was just as effective as HSVtk alone. EV refers to empty vector.

### Example 9

Intracranial delivery of viral particles was performed in mice. Referring to FIGS. 11A-11C, concentrated retrovirus, encoding the NLS-HSVtk-FIRE construct, was intracranially injected in mice harboring GBM tumors. After viral delivery, animals treated with GCV were euthanized and tissues were analyzed for successful delivery (GFP) and response to treatment (DNA damage marker pH2A. X). These data indicate that the construct can be administered intracranially and that there is sufficient ERK activity in vivo to mediate expression of NLS-HSVtk-FIRE and corresponding response to GCV.

### Example 10

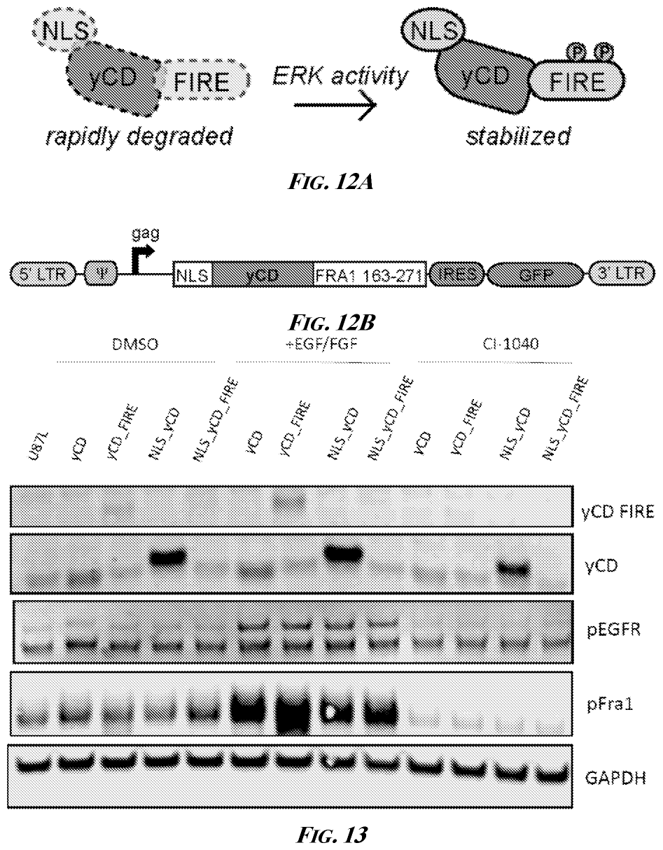

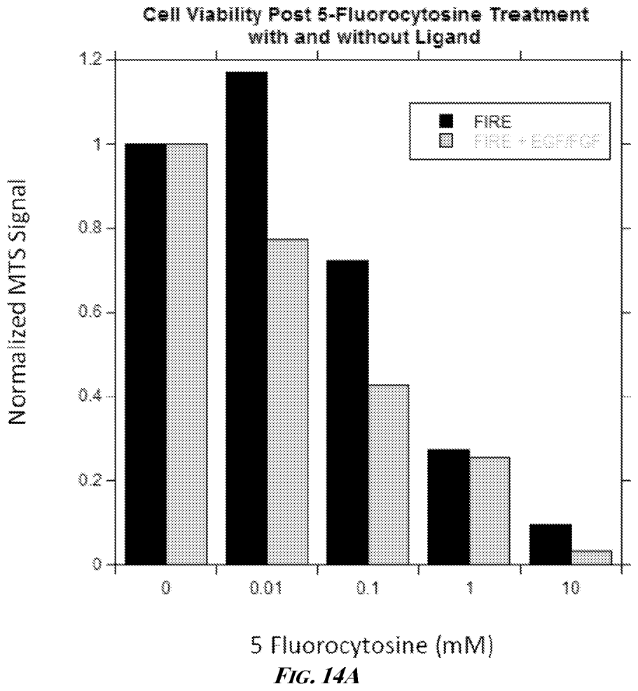

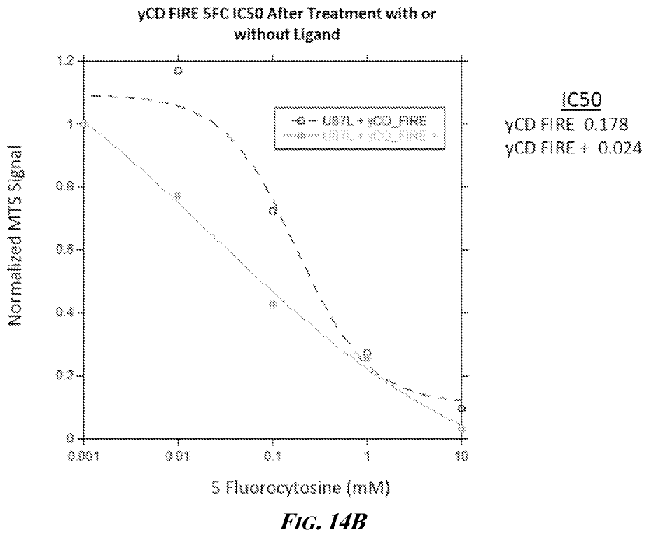

An alternative embodiment of the presently disclosed FIRE suicide gene system was prepared. Referring to FIGS. 12A and 12B, a yeast cytosine deaminase (yCD) was used with a nuclear localization sequence (NLS), and a FRA-1 integrated reporter element (FRA-1 PEST domain) (FIRE). The construct is used with 5-fluorocytosine (5-FC), a prodrug converted to a toxic product by yCD. The expression vector also includes a green fluorescence protein (GFP) sequence. Phosphorylation of the FIRE domain by ERK stabilizes yCD-FIRE fusion protein expression, creating an alternative ERK-selective suicide gene product.

### Example 11

Referring to FIG. 13, it was shown that yCD expression can be stabilized by growth factors that drive ERK activity or antagonized by MEK inhibition. In the cell lines engineered thus far, the effects are most apparent in those expressing the NLS-yCD version of the fusion protein.

### Example 12

It was shown that ERK activity promotes cell killing in response to 5-FC in cells expressing yCD-FIRE. Referrring to FIGS. 14A and 14B, cell viability decreases in response to 5-FC plus growth factors that drive ERK activity. Cells treated with growth factors that drive ERK activity have a lower 5-FC IC50 than cells not treated with growth factor.

### Example 13

This EXAMPLE is based on the activity of the p38 MAP kinase pathway for controlling the expression of the herpes simplex virus thymidine kinase (HSVtk), a common suicide gene product that converts the prodrug ganciclovir into a toxic phosphorylated form. Because p38 is activated in response to chemotherapy-induced DNA damage and other cell stresses, our design can combine synergistically with chemotherapy to create a positive feedback loop that leads to enhanced tumor cell killing. In the design presented, HSVtk was fused with peptide sequences whose turnover was predicted to be slowed when phosphorylated by p38. Four such peptides were created using PEST domains (sequences rich in proline, glutamic acid, serine, and threonine that regulate protein turnover) from the MAP kinase substrates CHOP, MEF2A, p21, and FRA1 as starting points. See FIGS. 15-20. Retroviral expression vectors encoding these novel suicide genes were cloned and used to transduce a human glioma-initiating cell line. The present disclosure describes the selectivity of these constructs for p38-dependent cell killing and the cooperativity of these novel suicide genes with temozolomide chemotherapy. More broadly, this EXAMPLE demonstrates the utility of post-translational control of suicide gene activity as an alternative to transcriptional regulation.

### Example 14

G816-FLAG-NLS-HSVtk-PEST were grown in hypoxia (1% oxygen) for three days and treated with SB203580 (p38i, 10 μM), treated with temozolomide (TMZ, 100 μM), or transfected with control or p38 siRNA, then treated with temozolomide (TMZ, 100 μM) for two days. The results are shown in FIGS. 26A-26C.

### Example 15

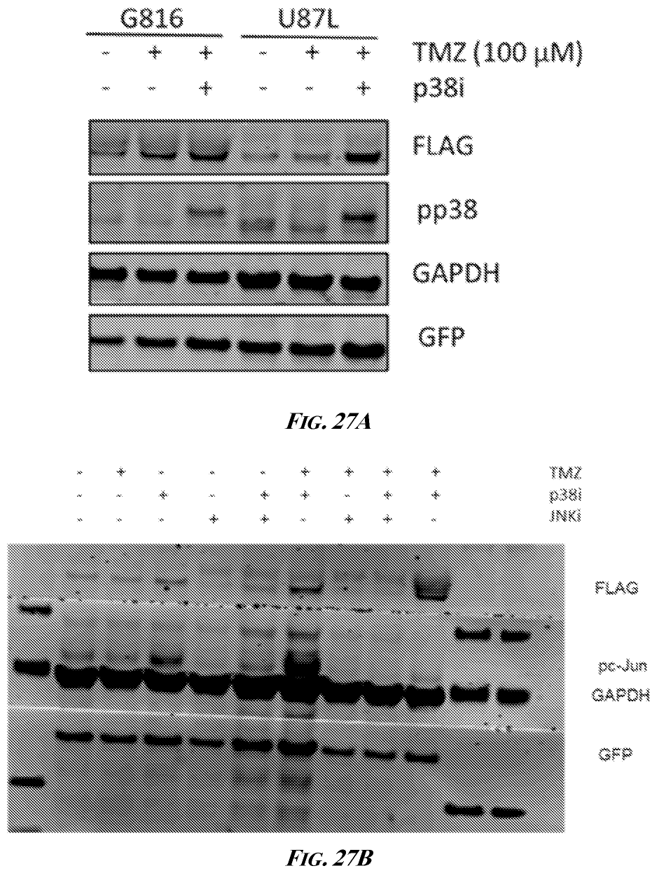

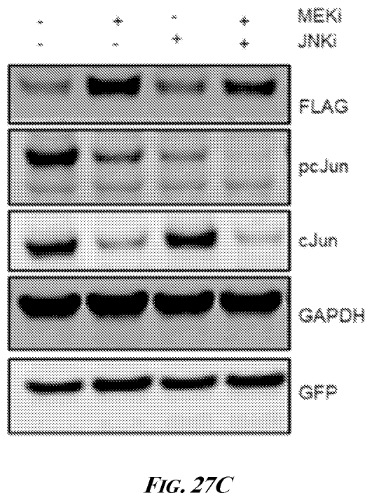

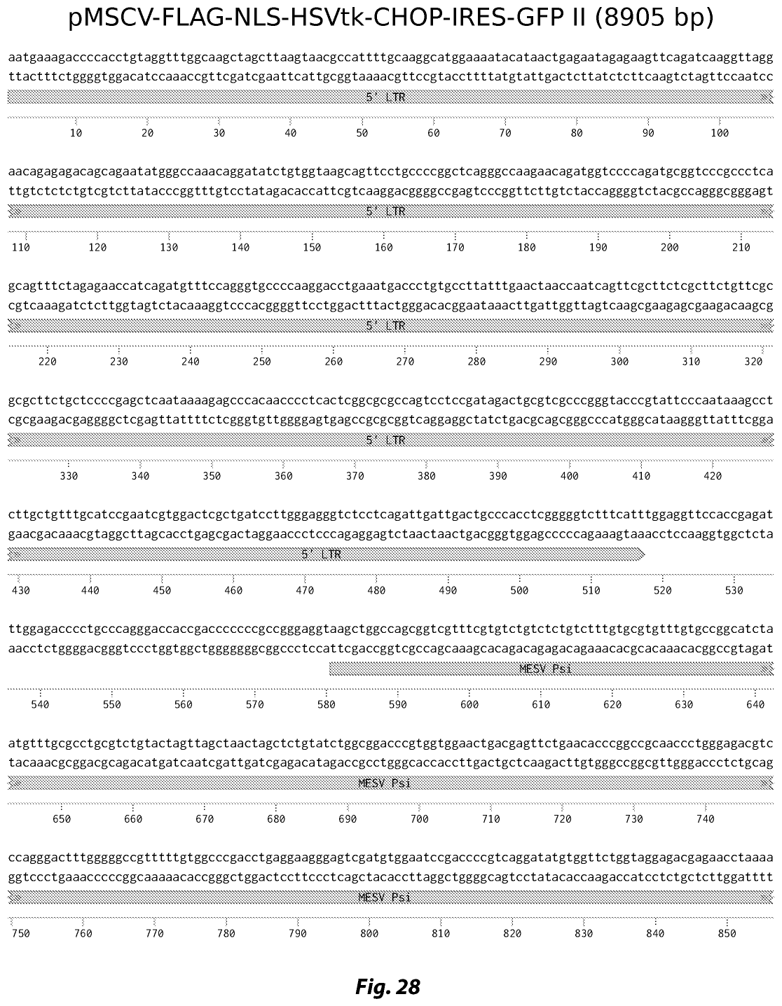

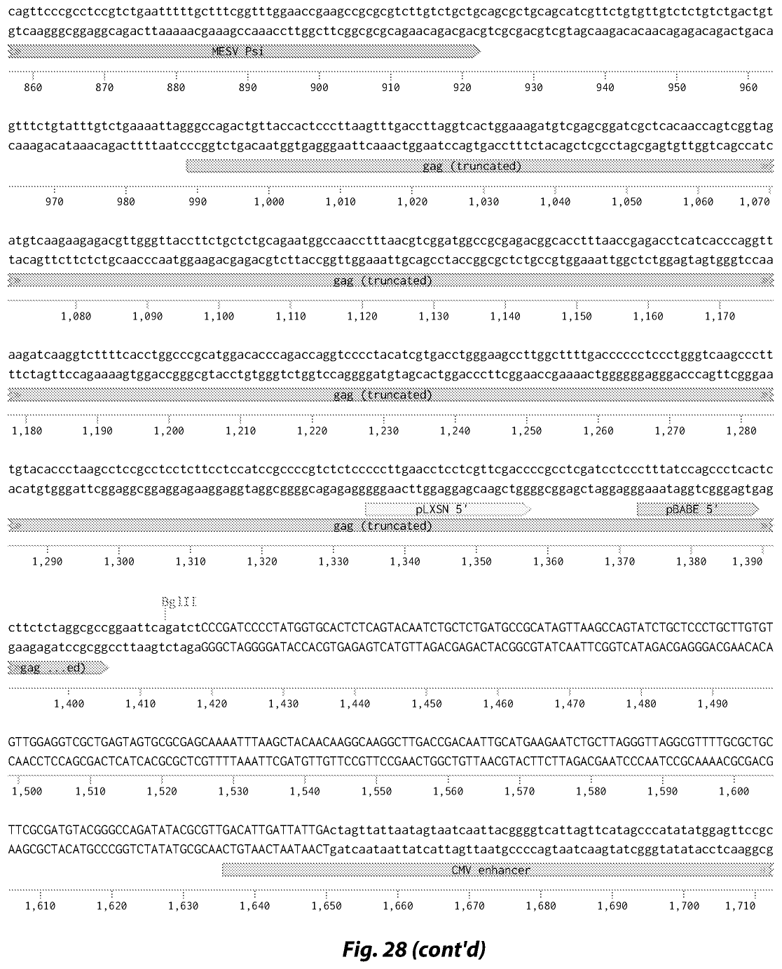

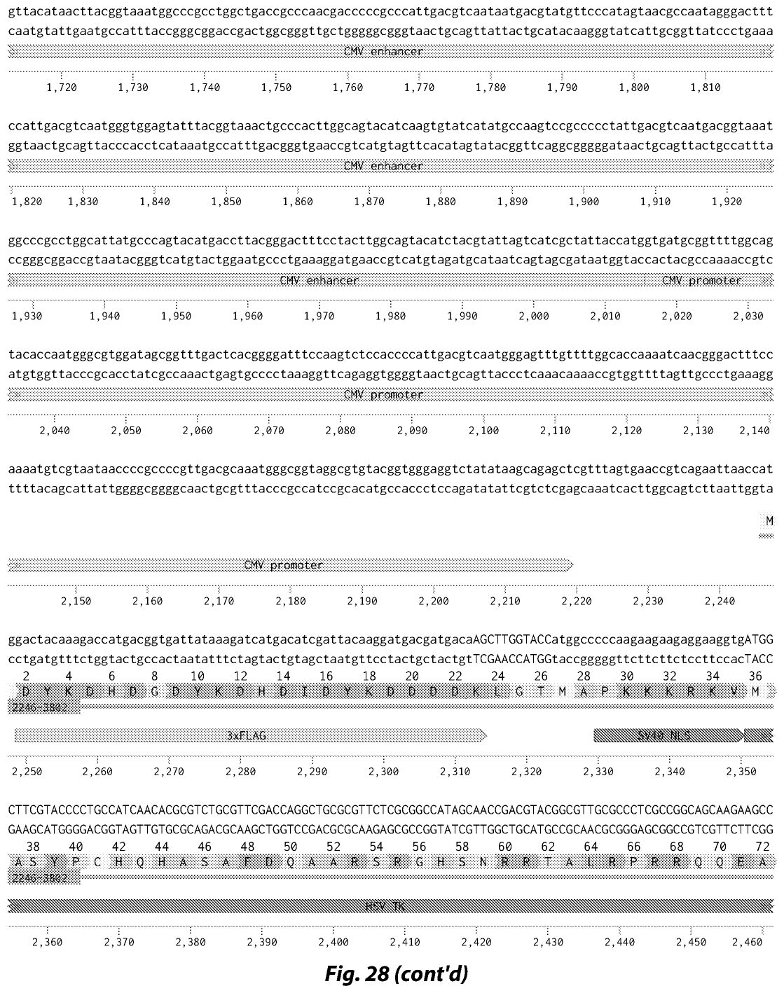

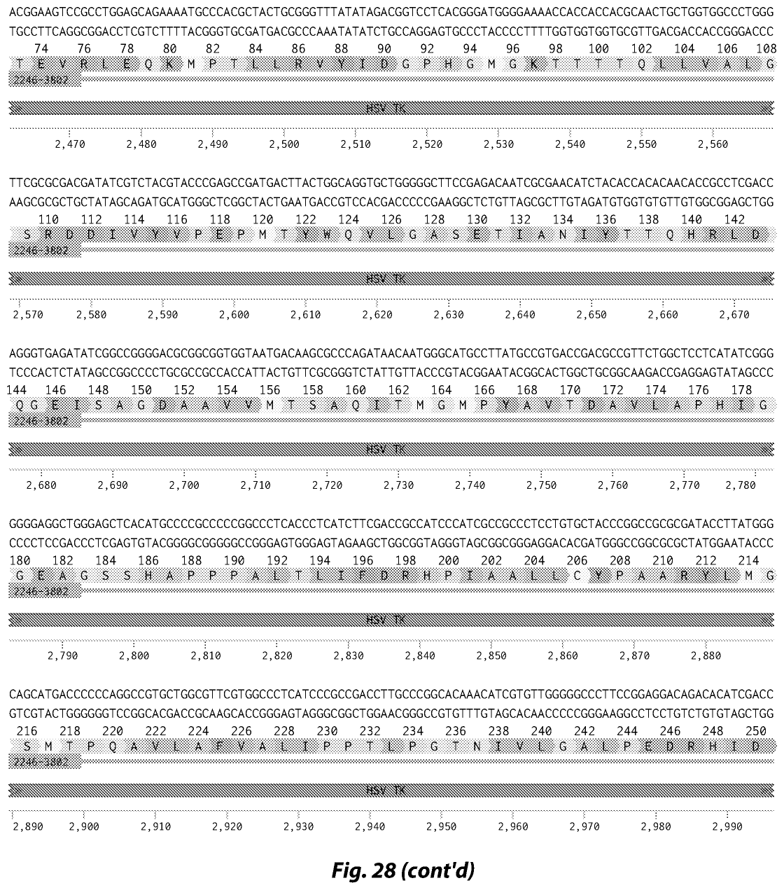

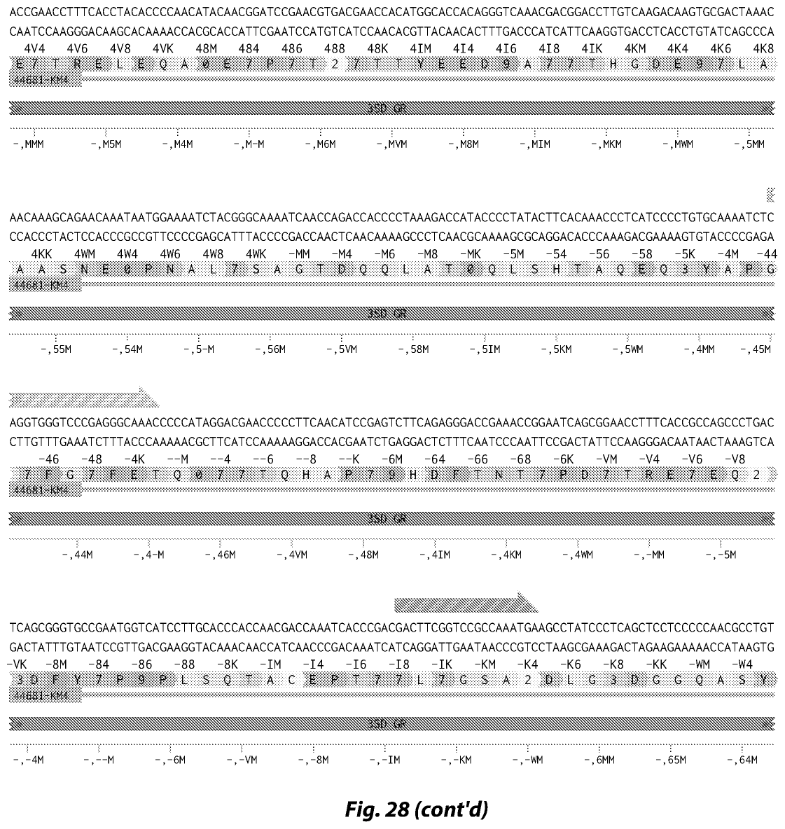

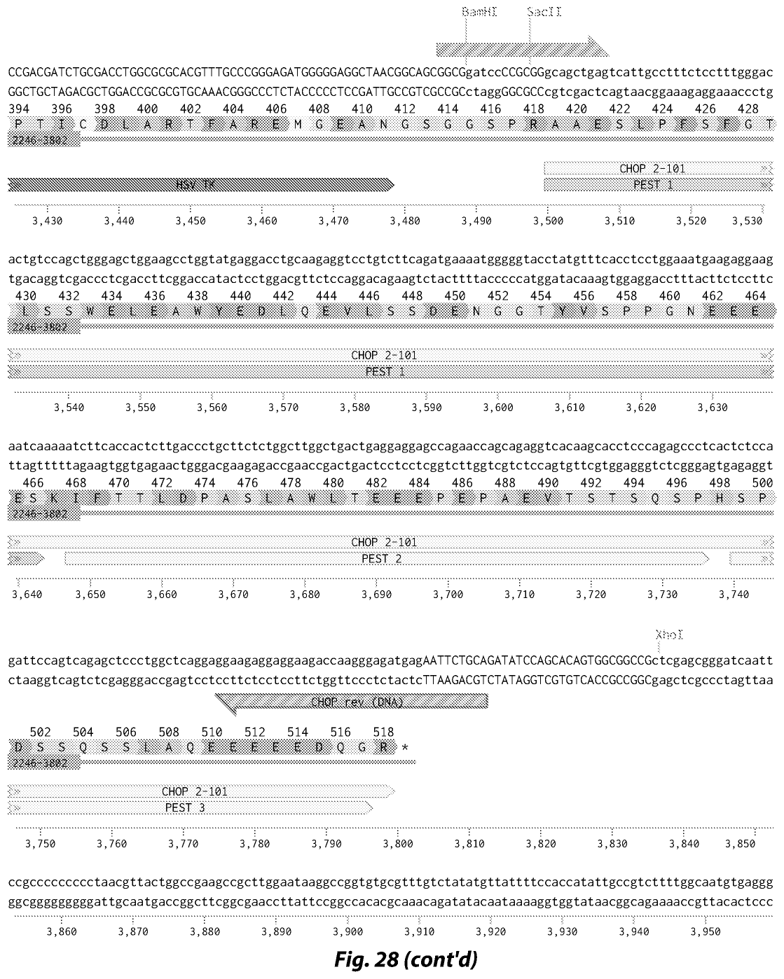

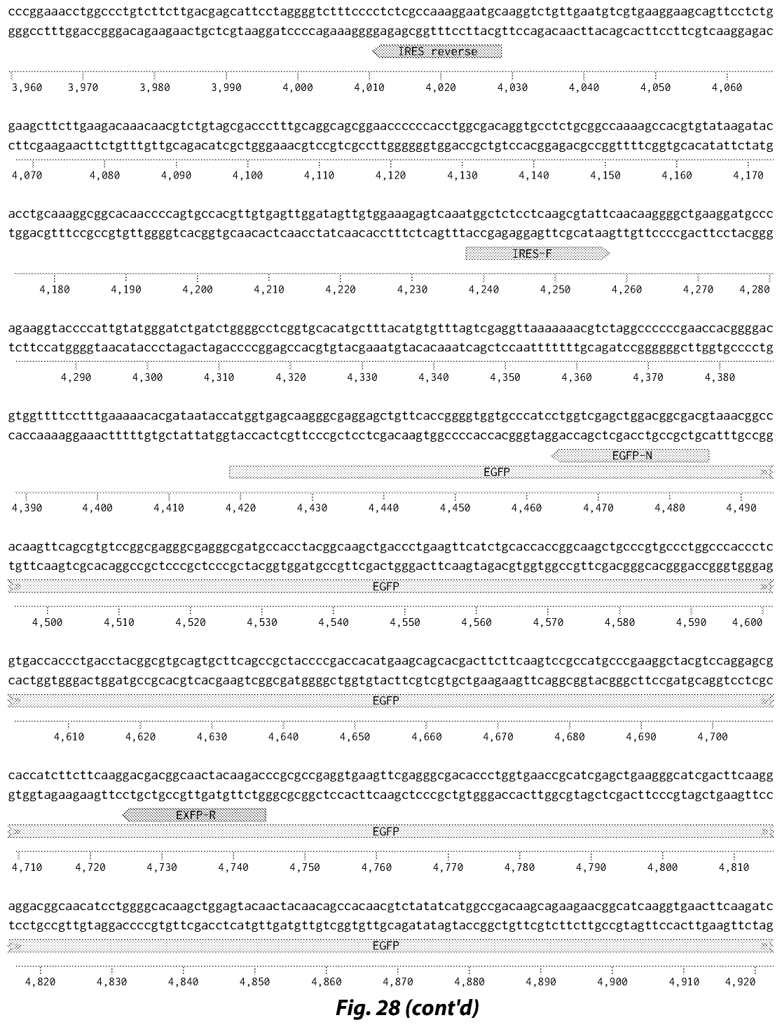

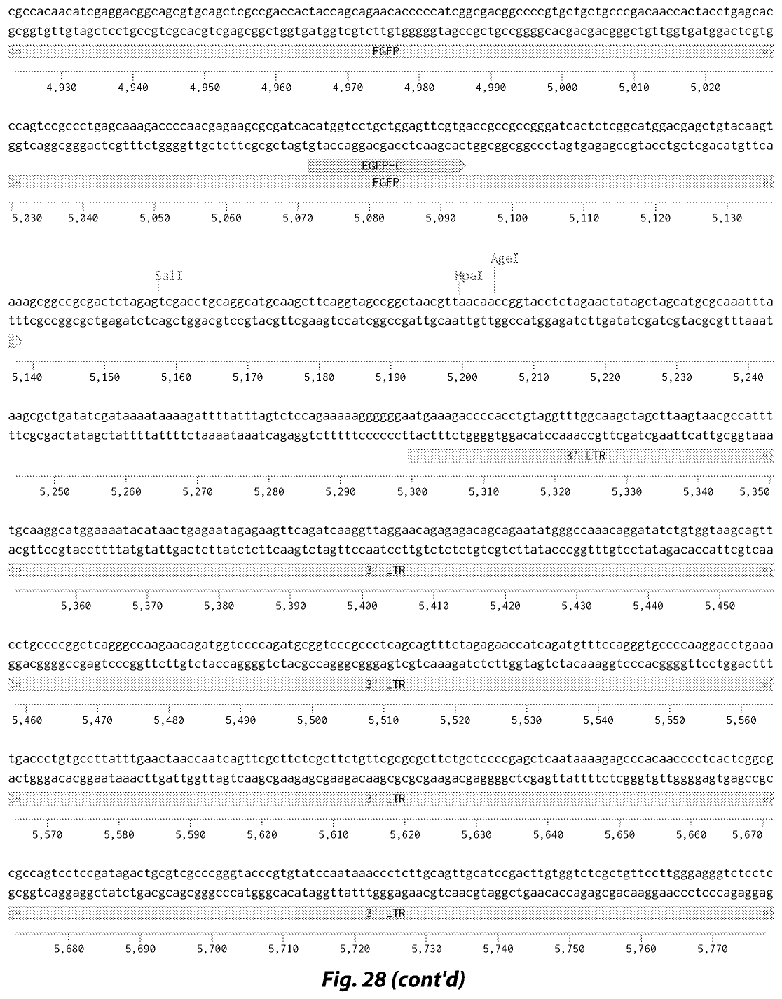

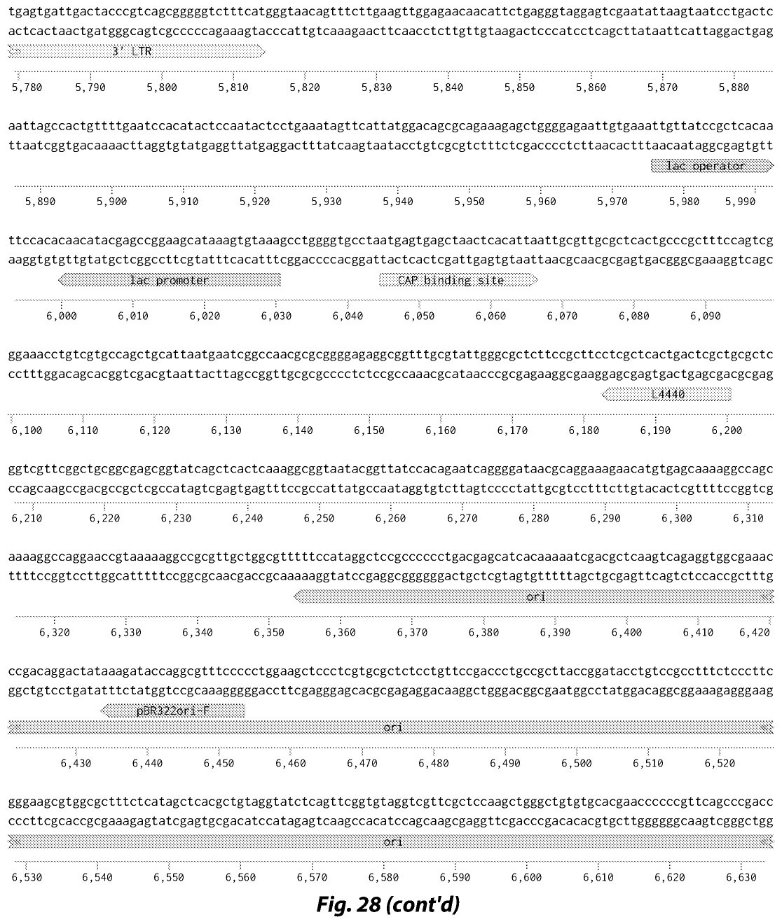

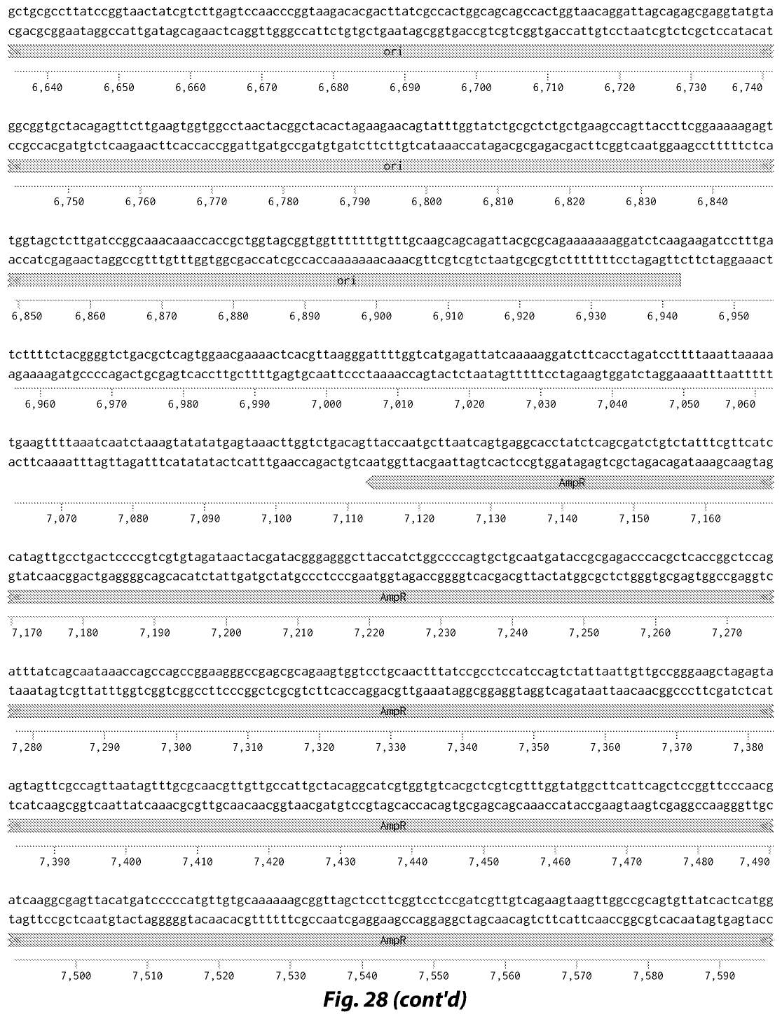

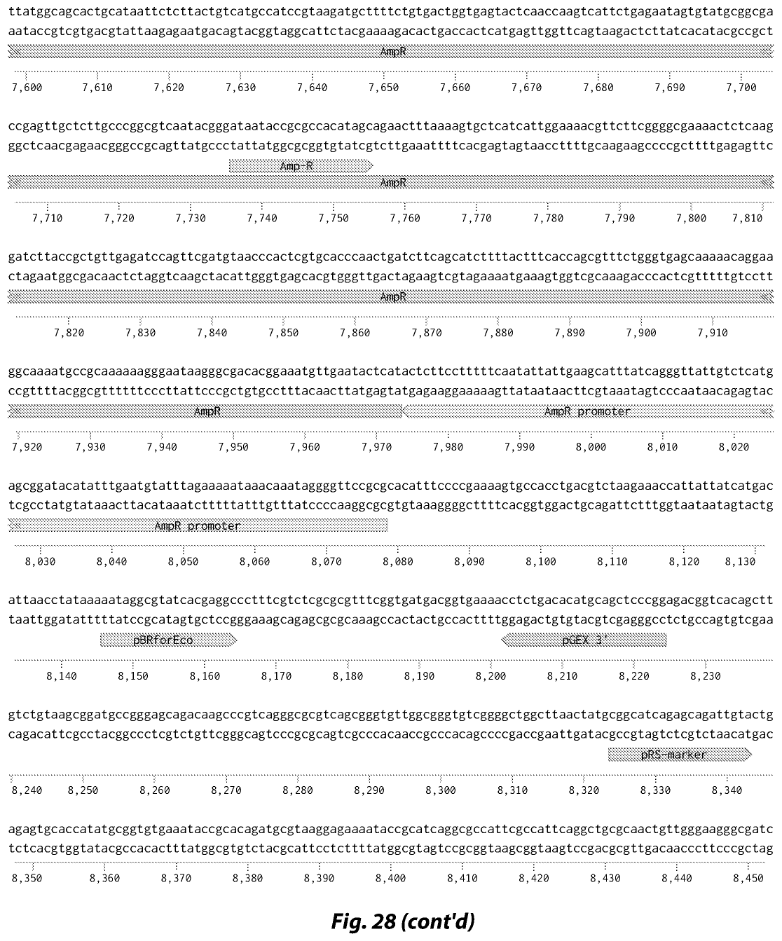

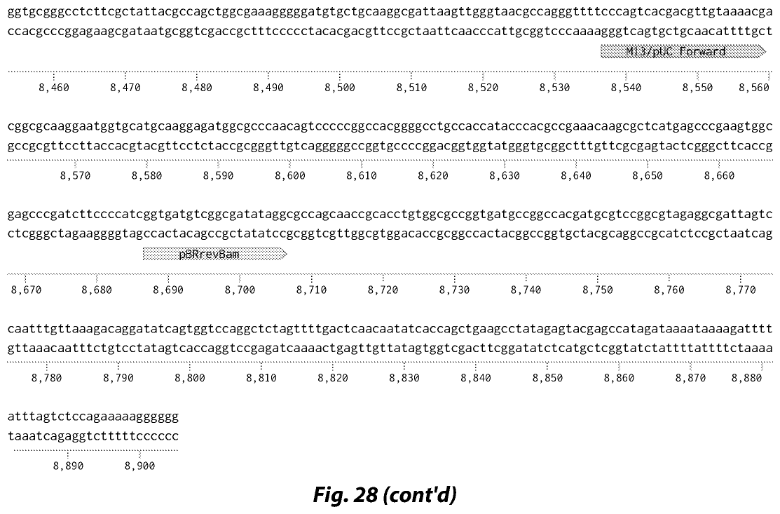

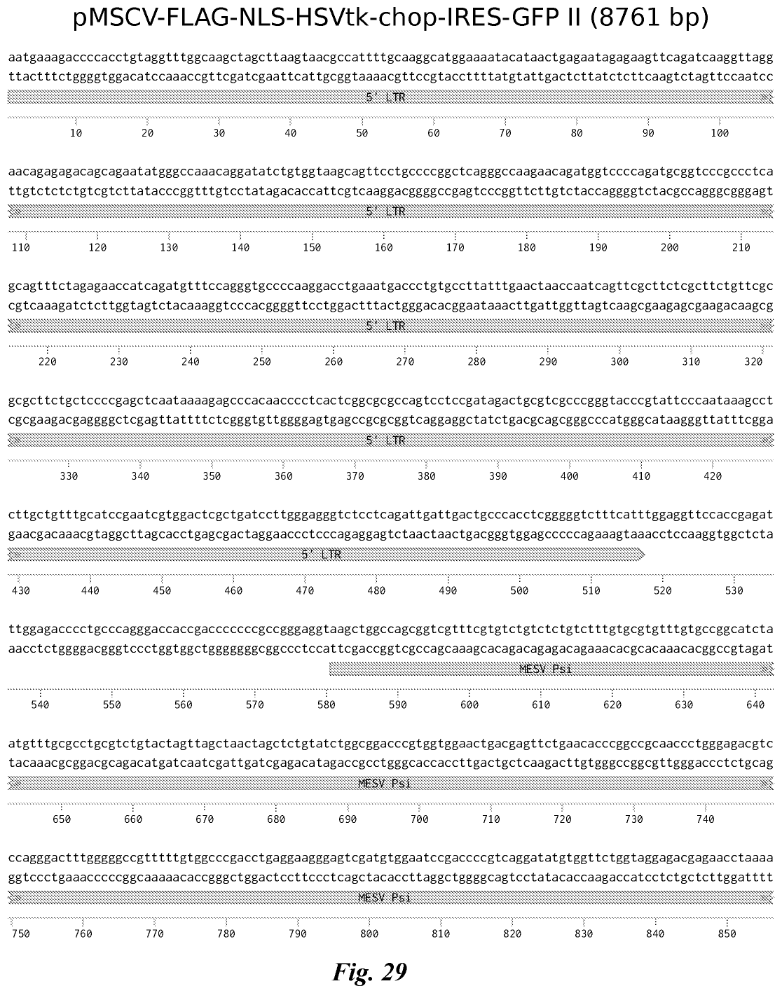

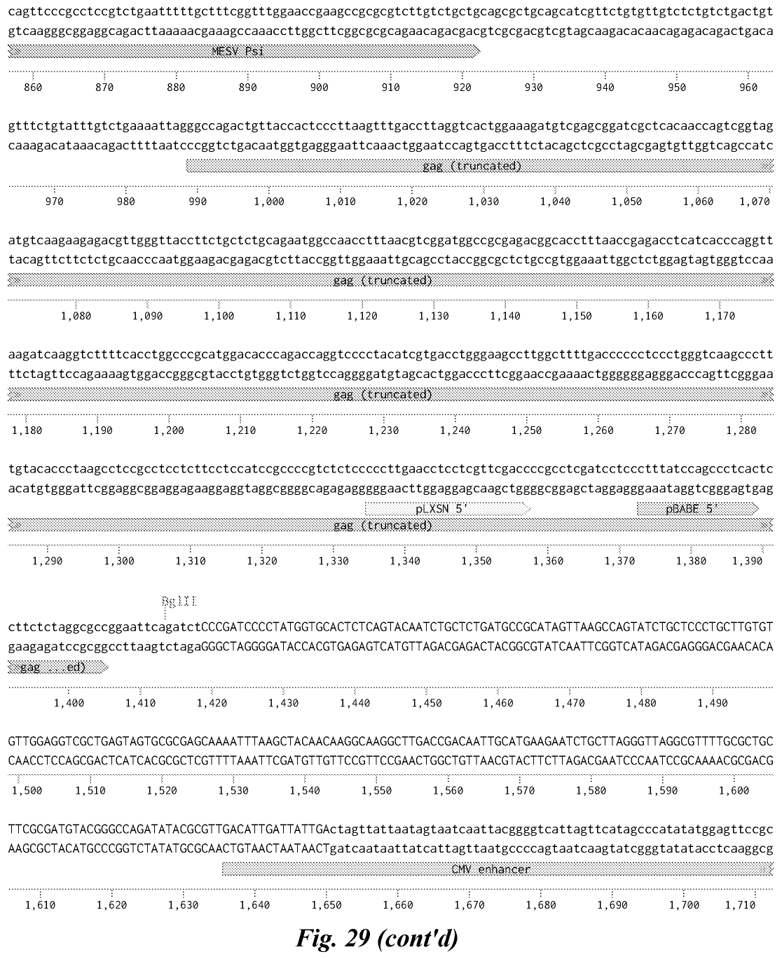

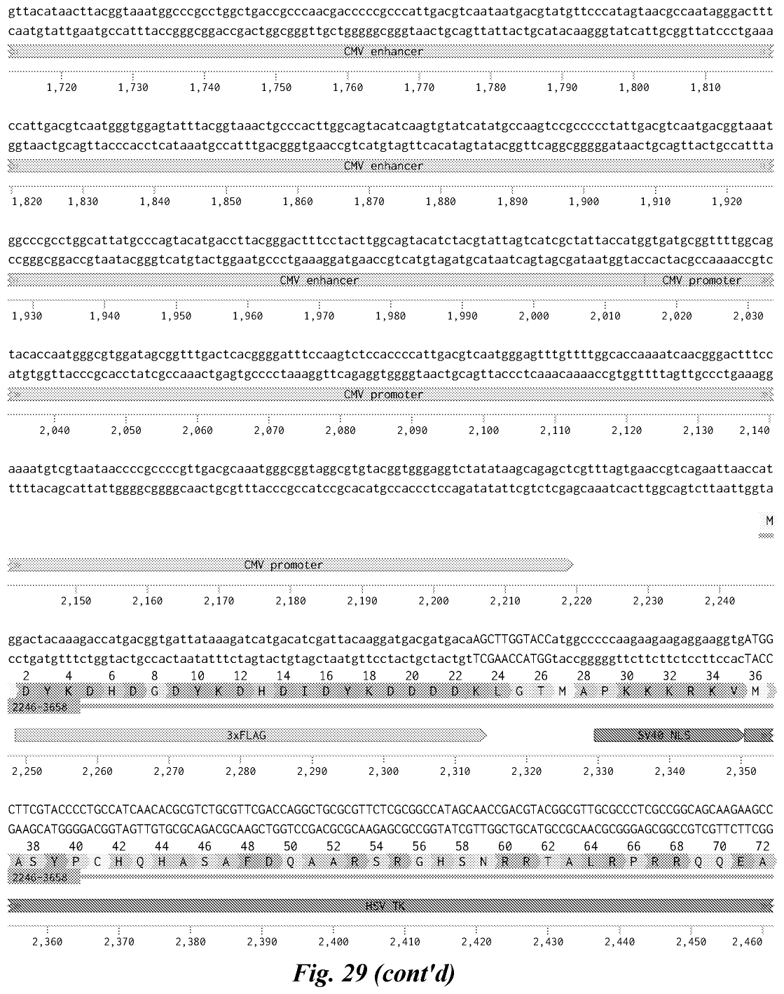

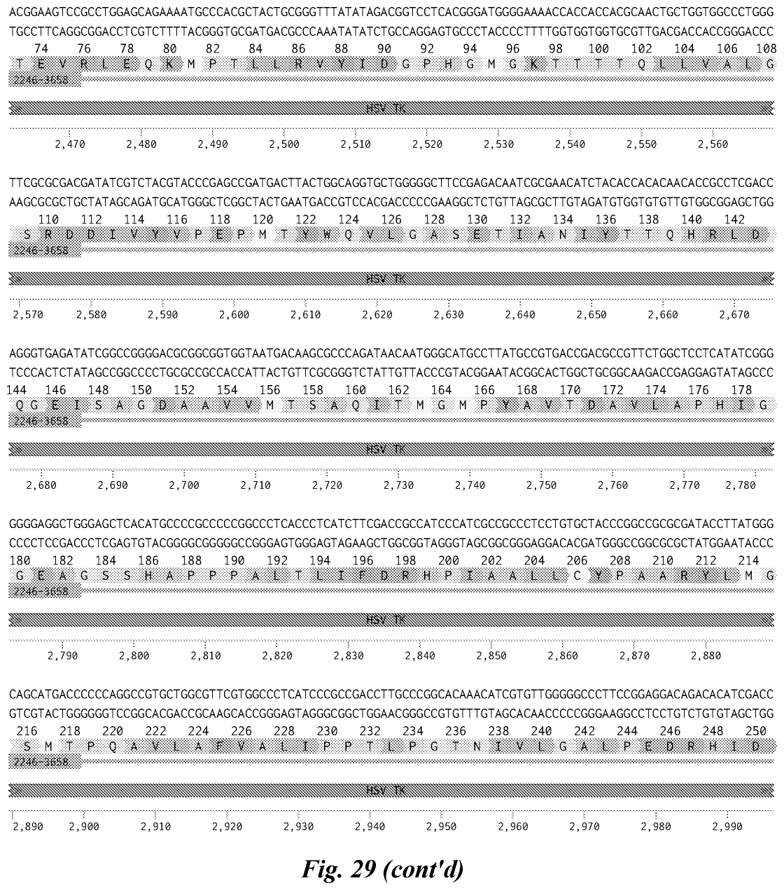

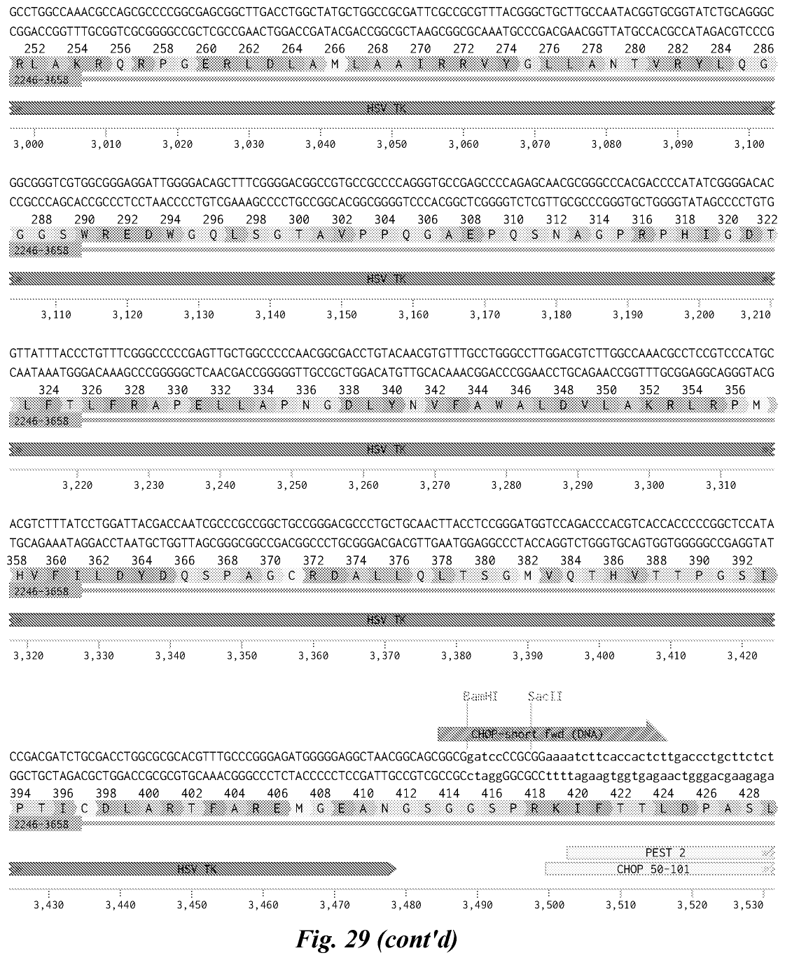

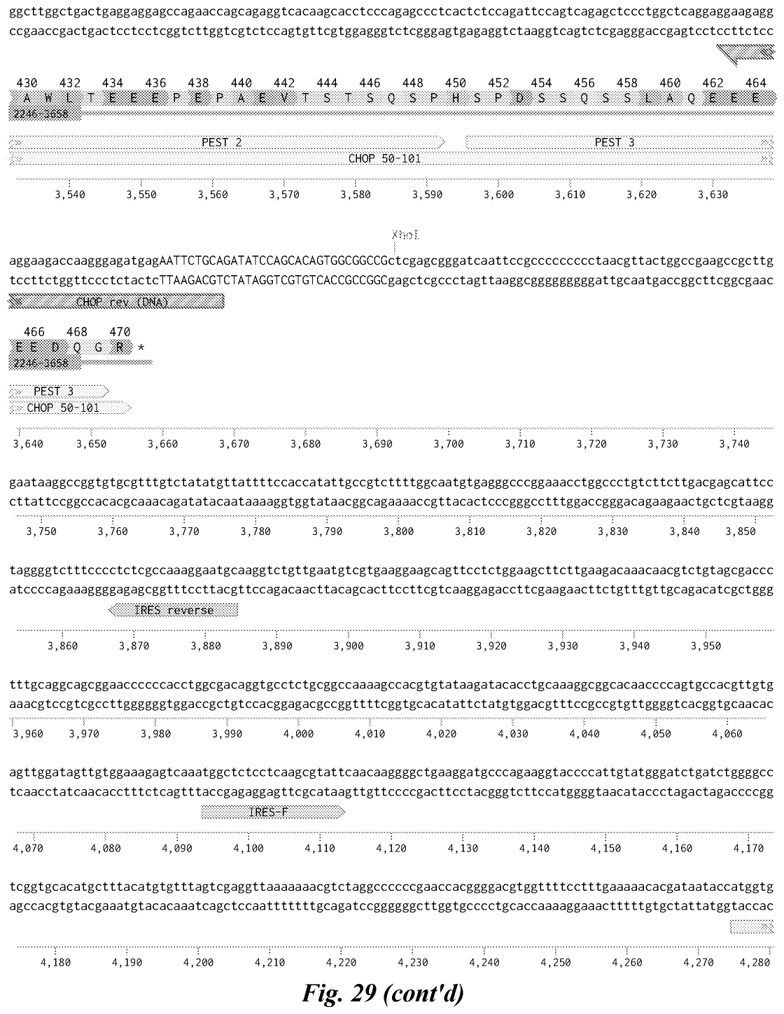

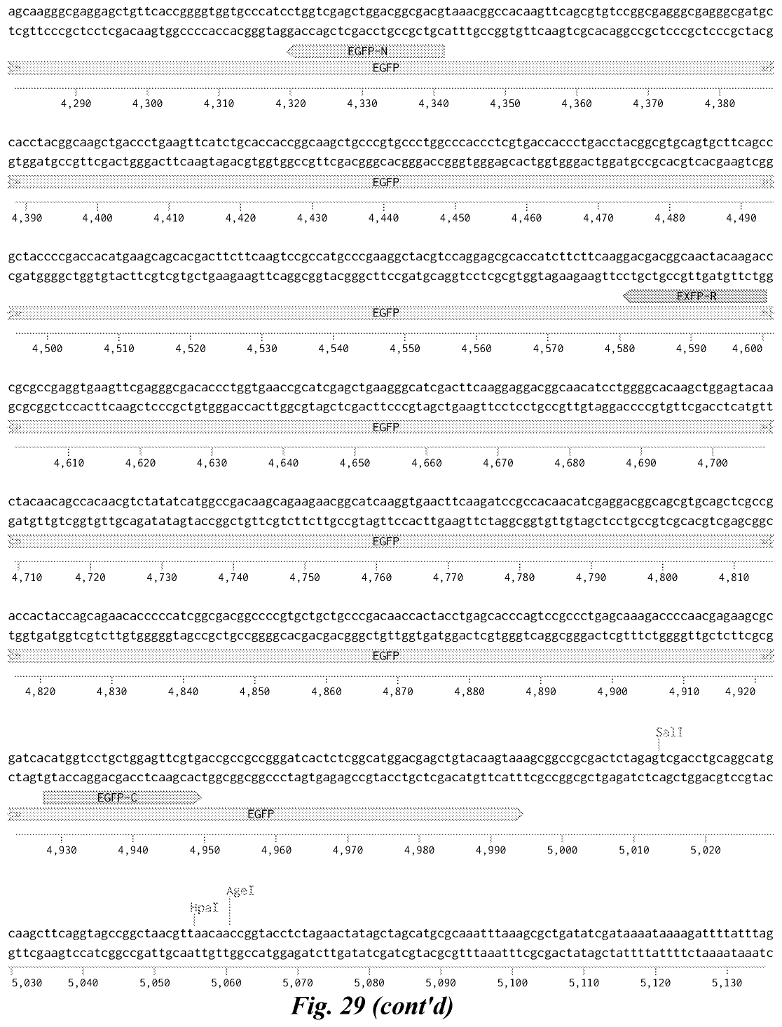

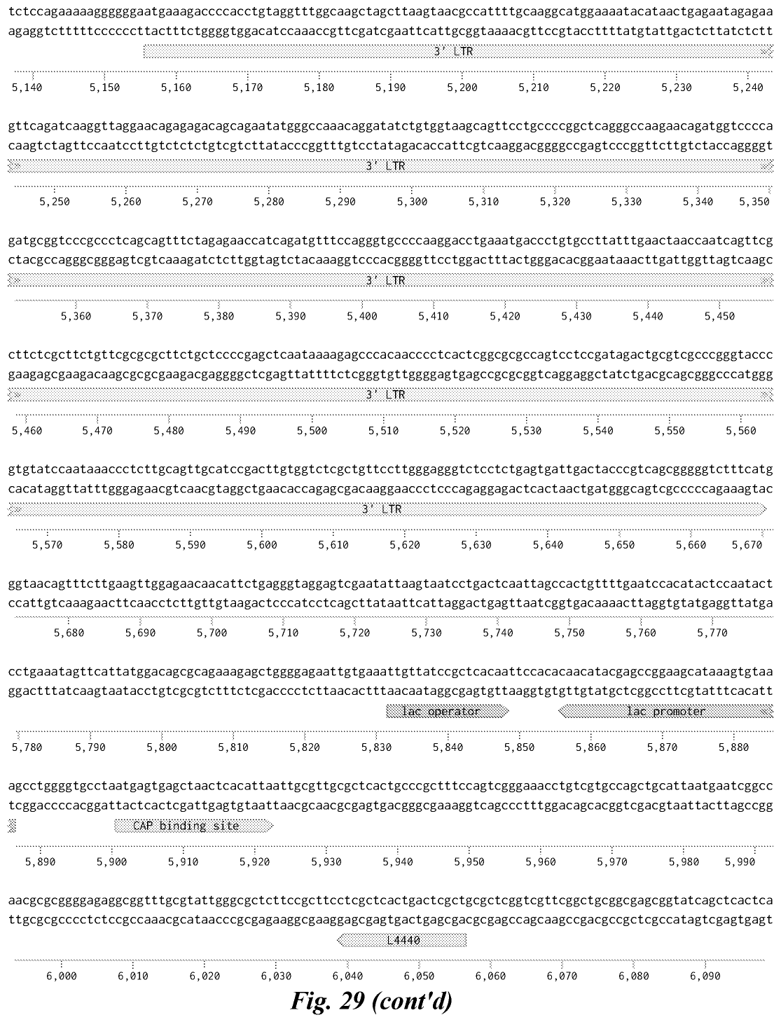

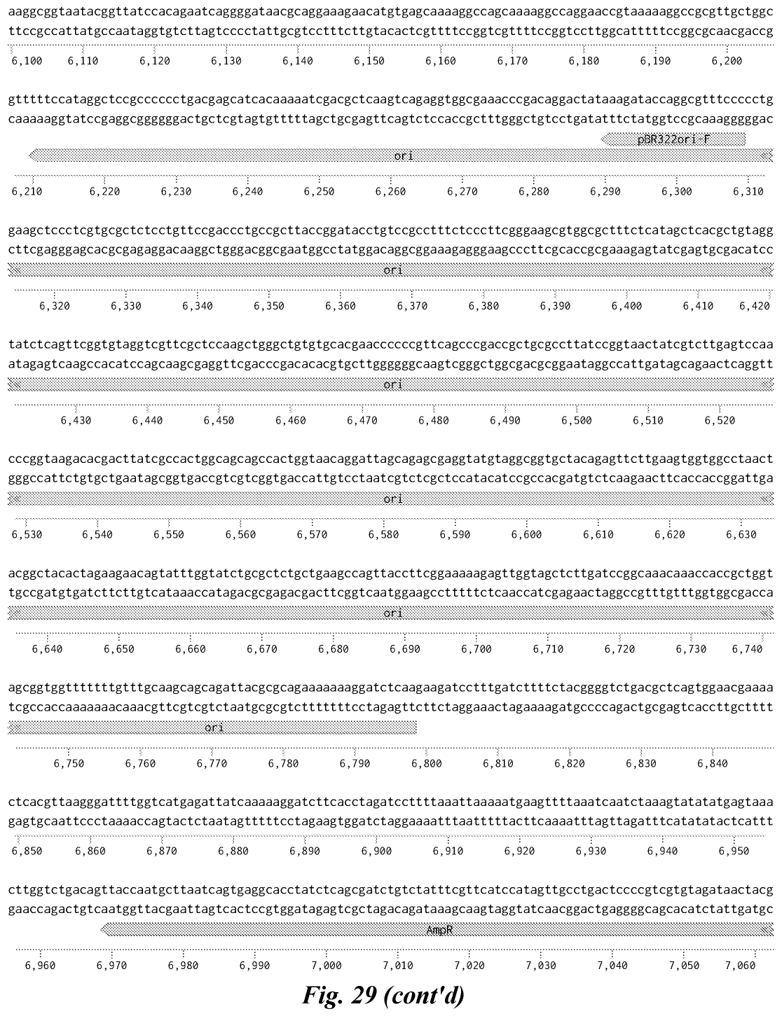

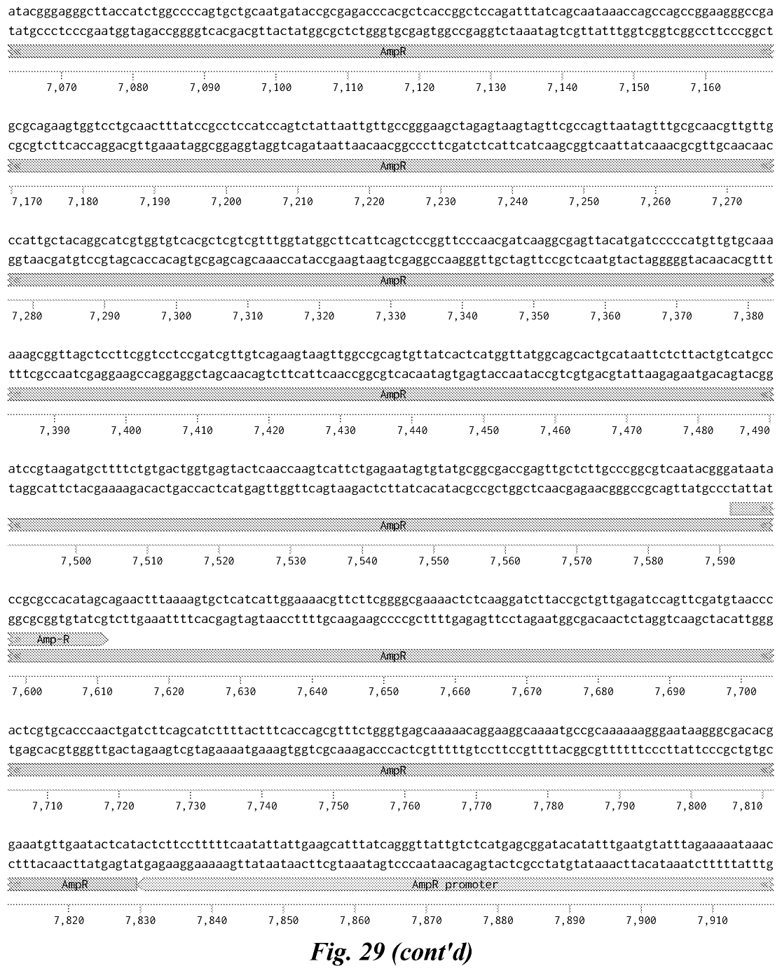

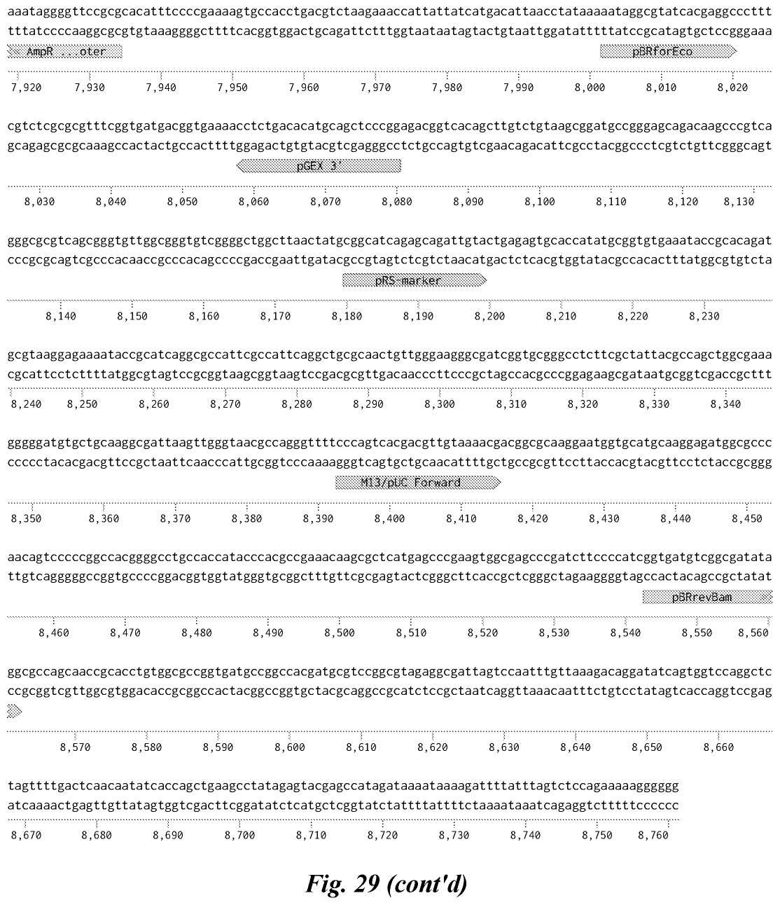

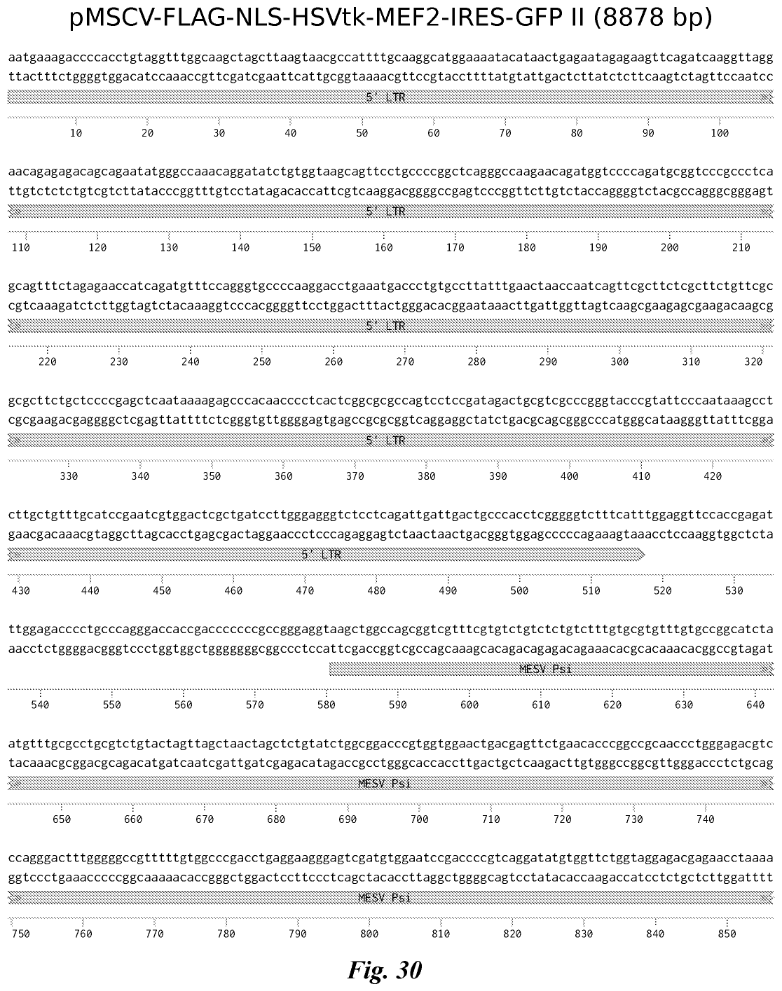

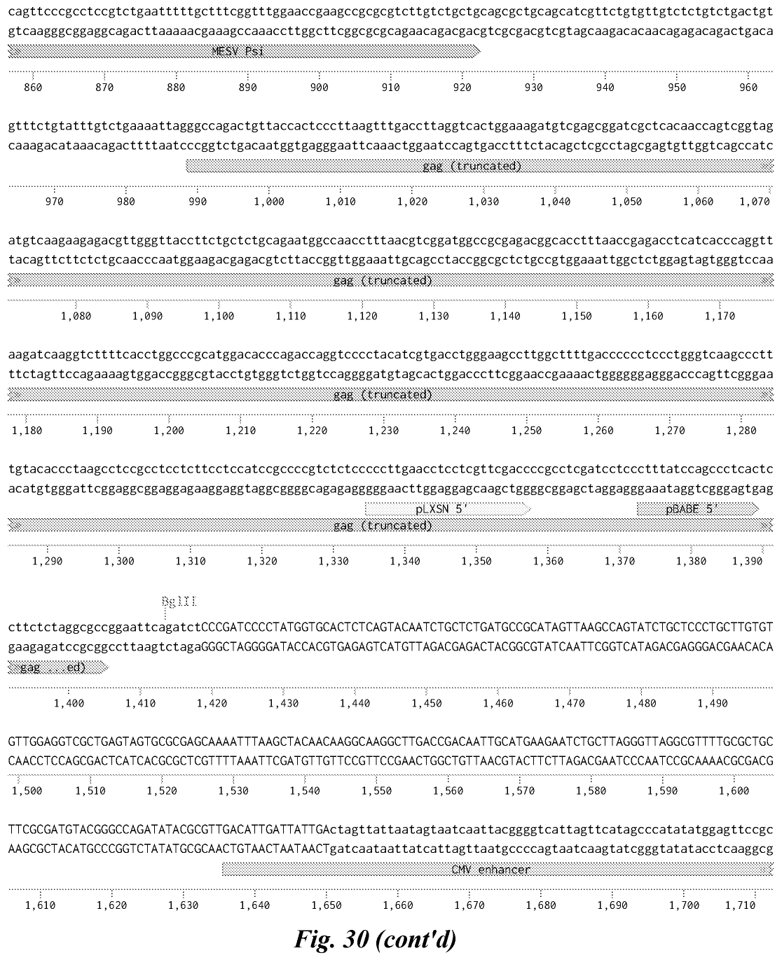

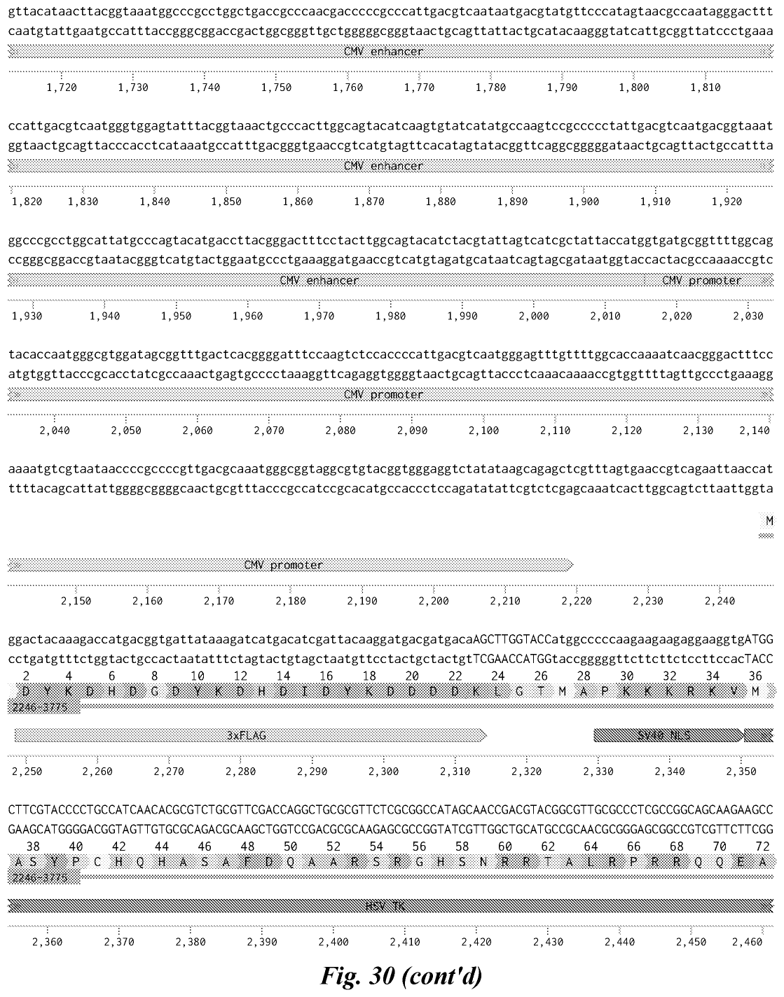

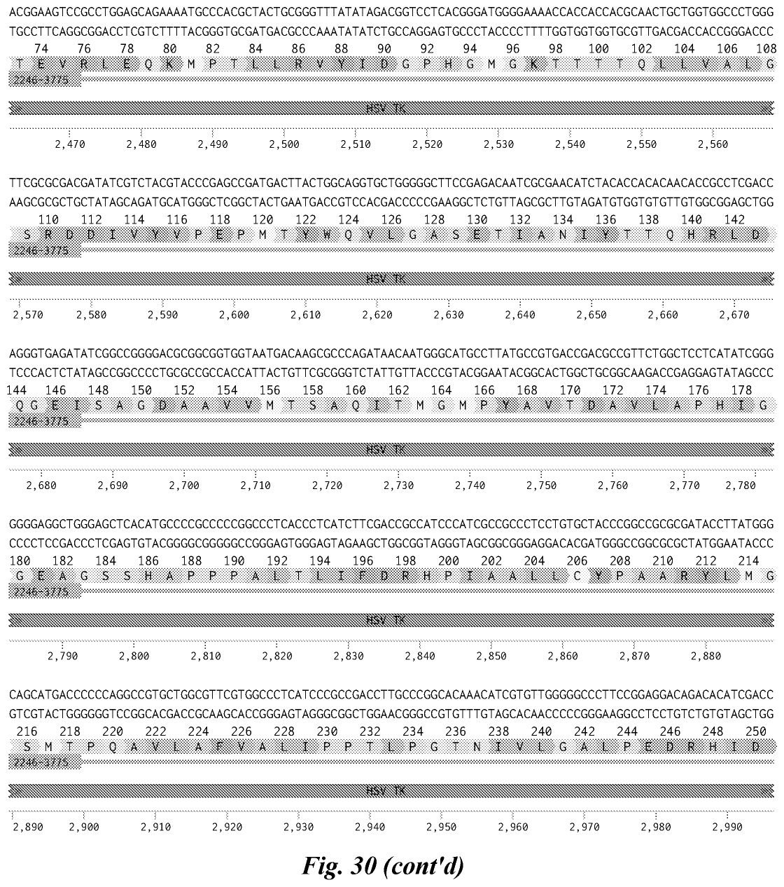

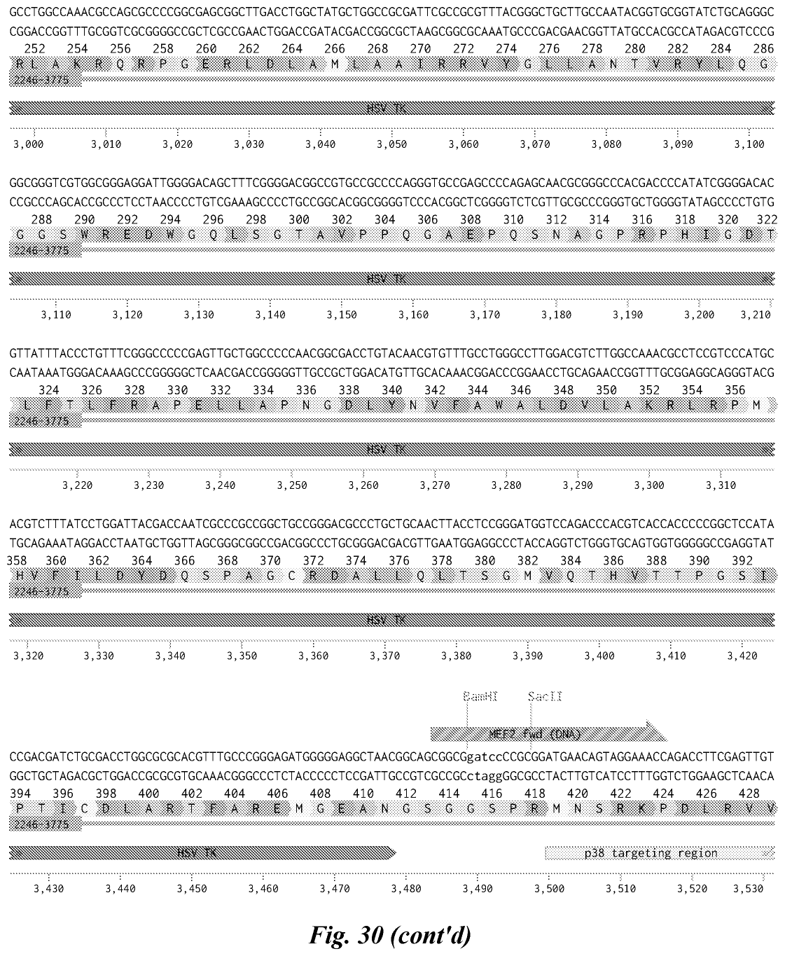

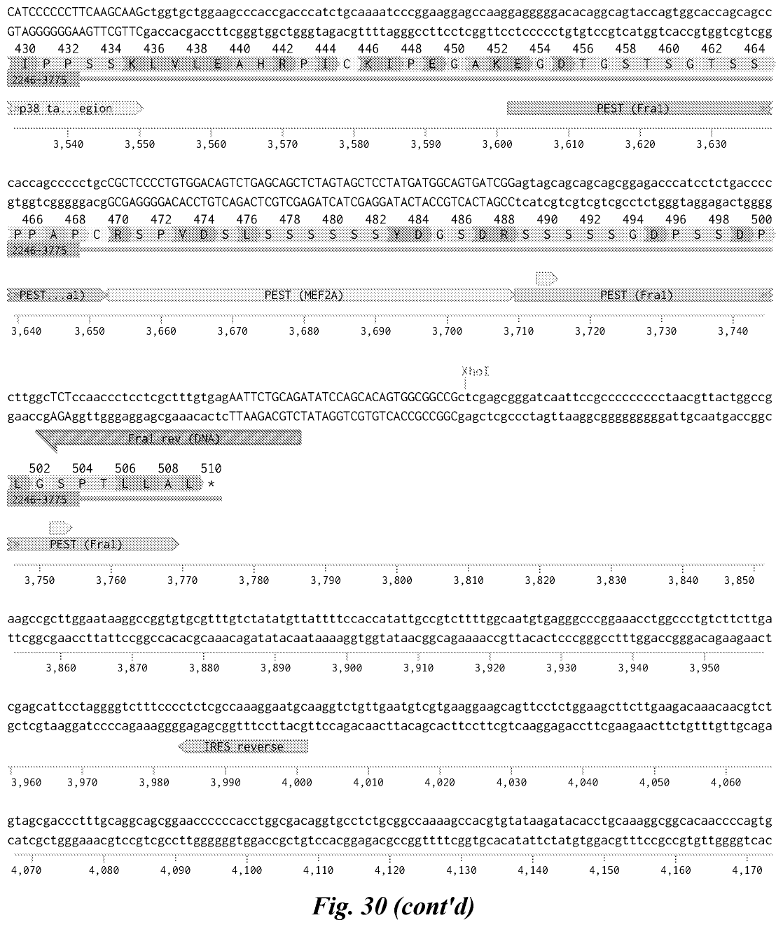

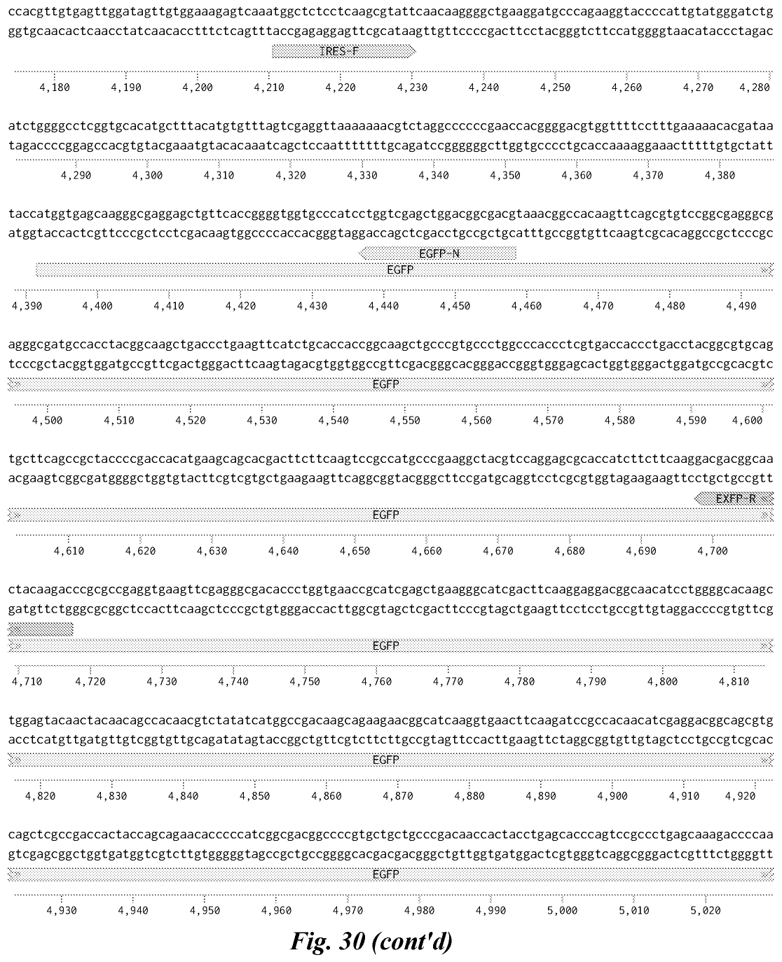

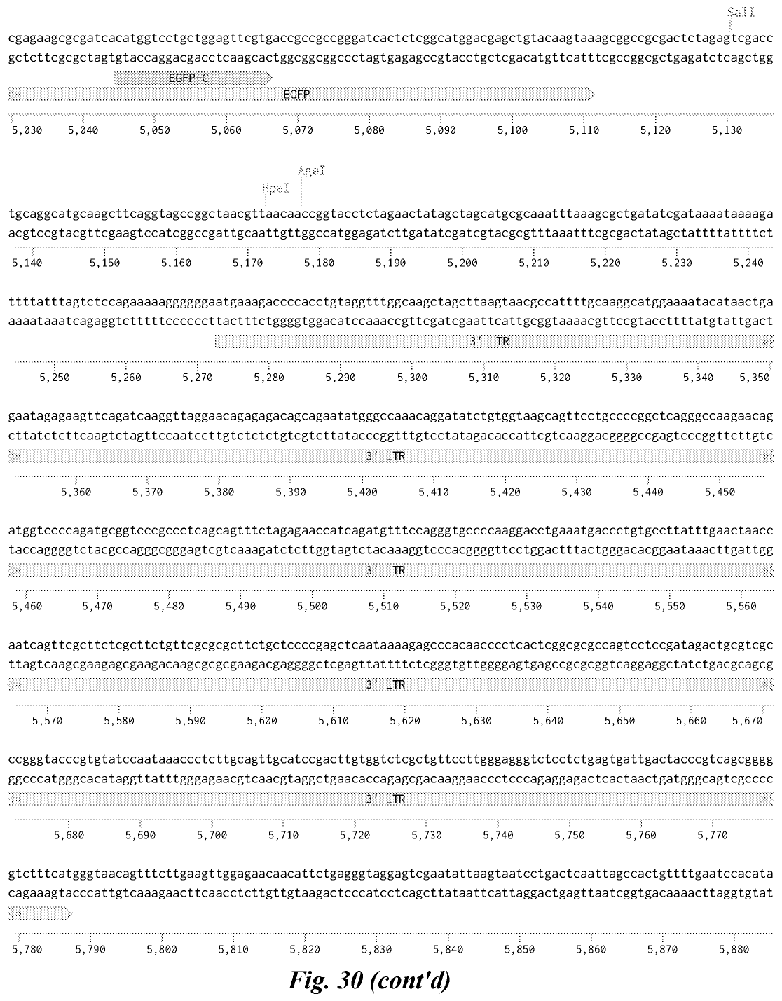

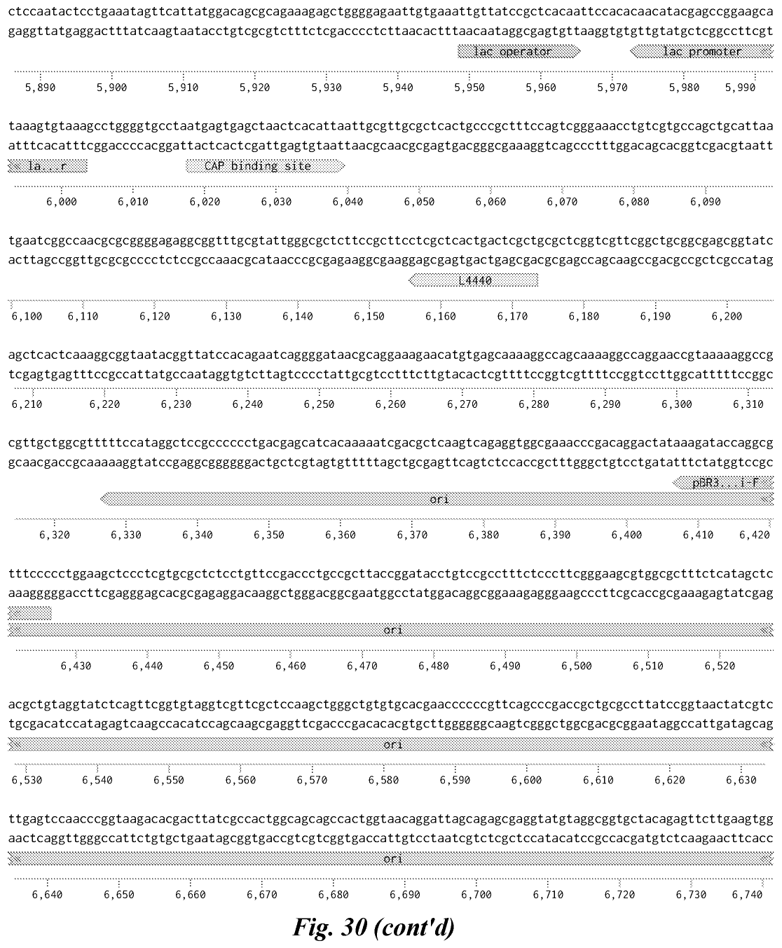

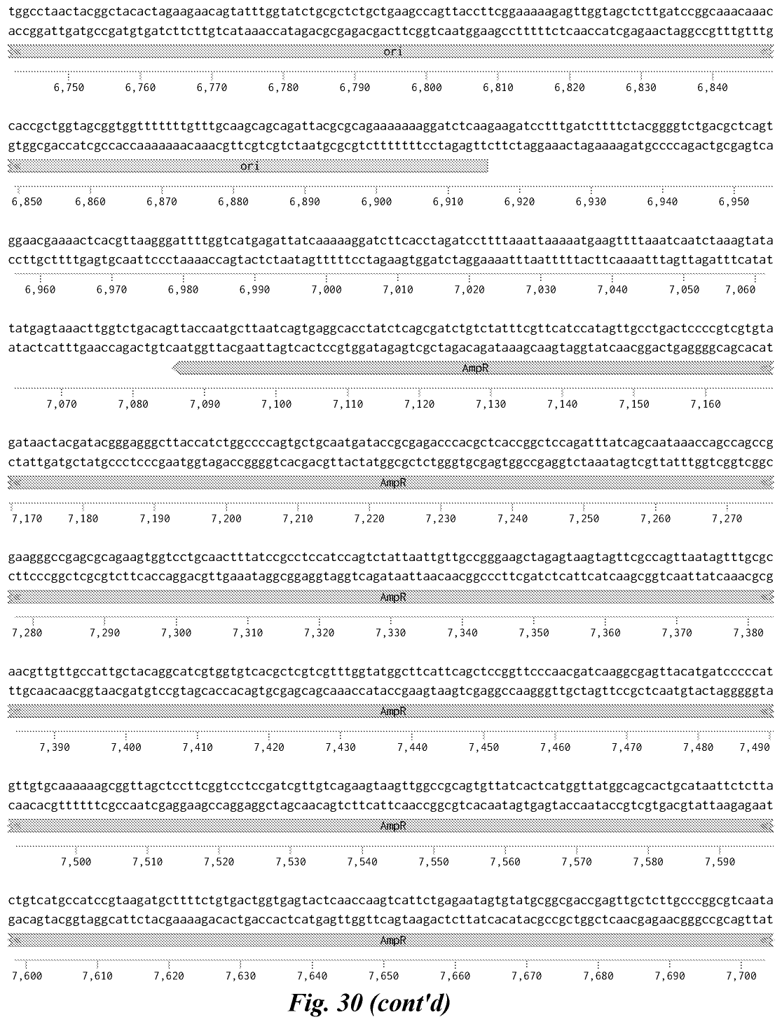

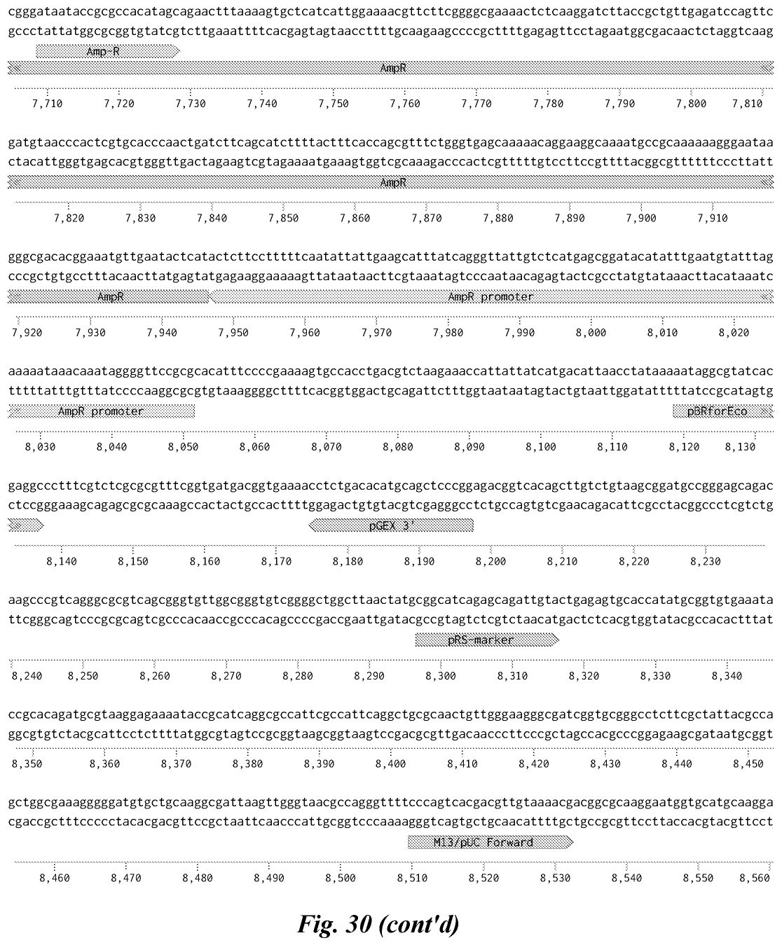

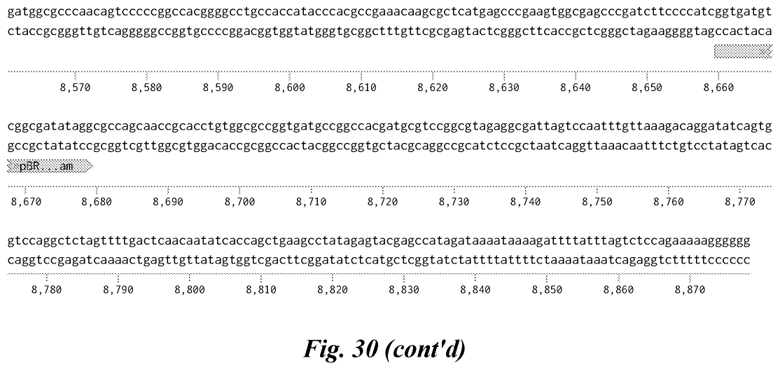

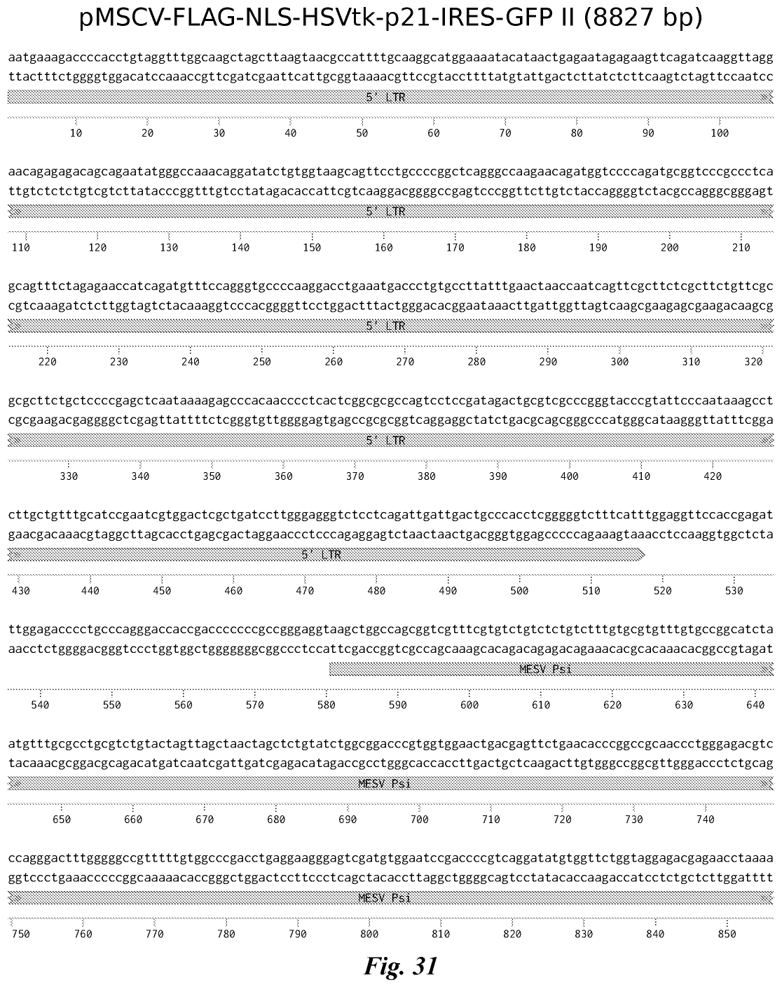

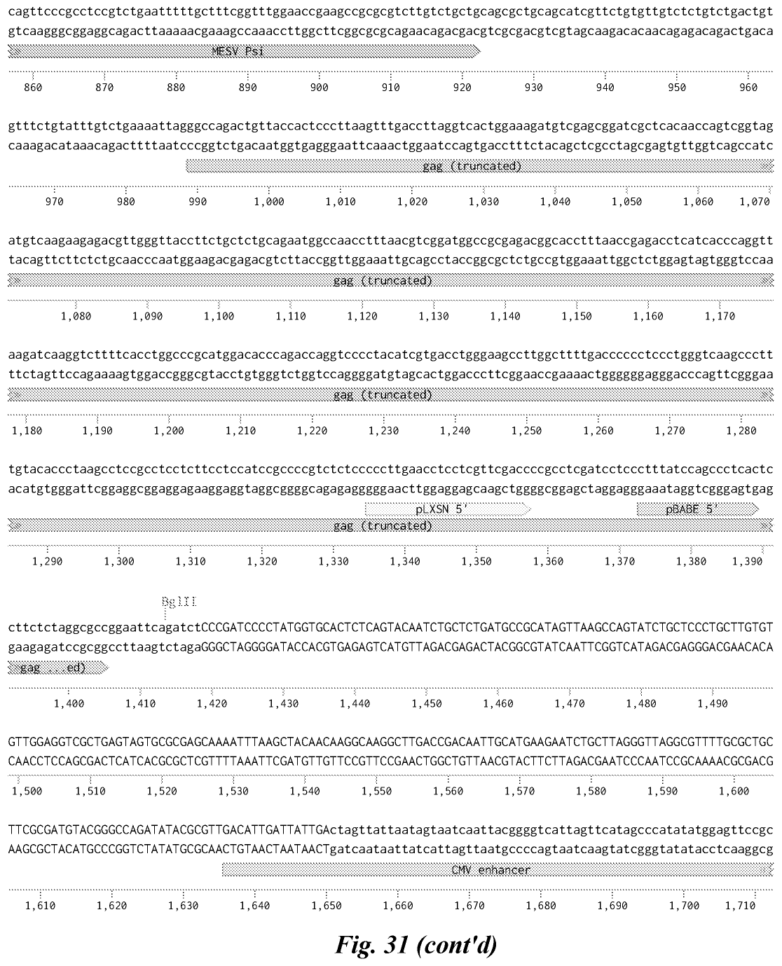

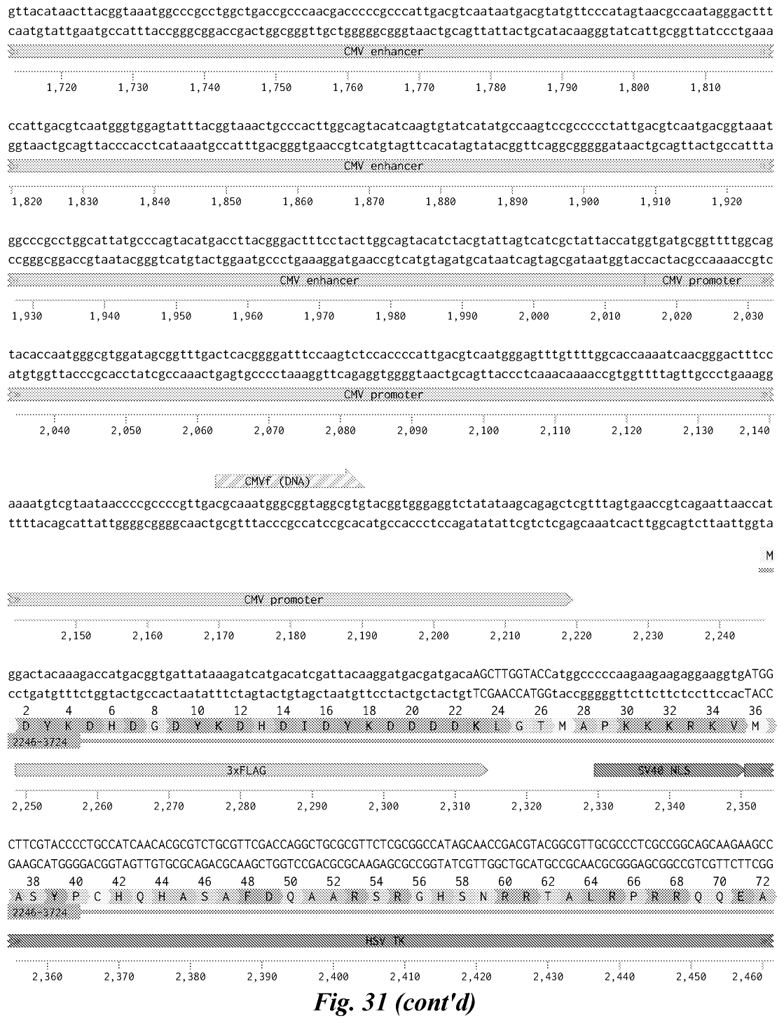

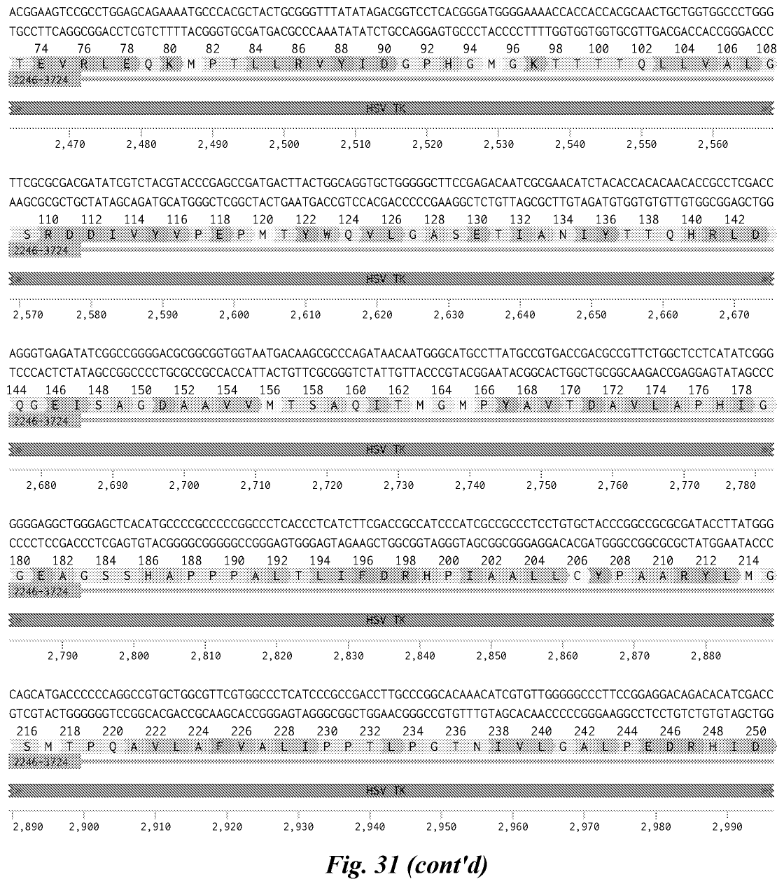

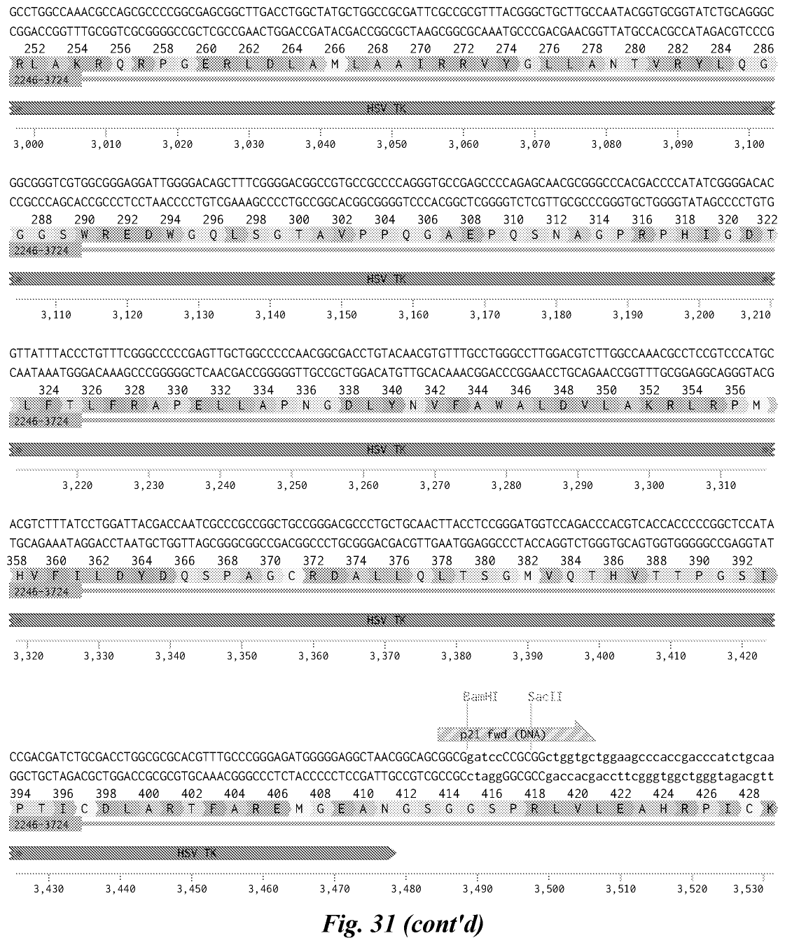

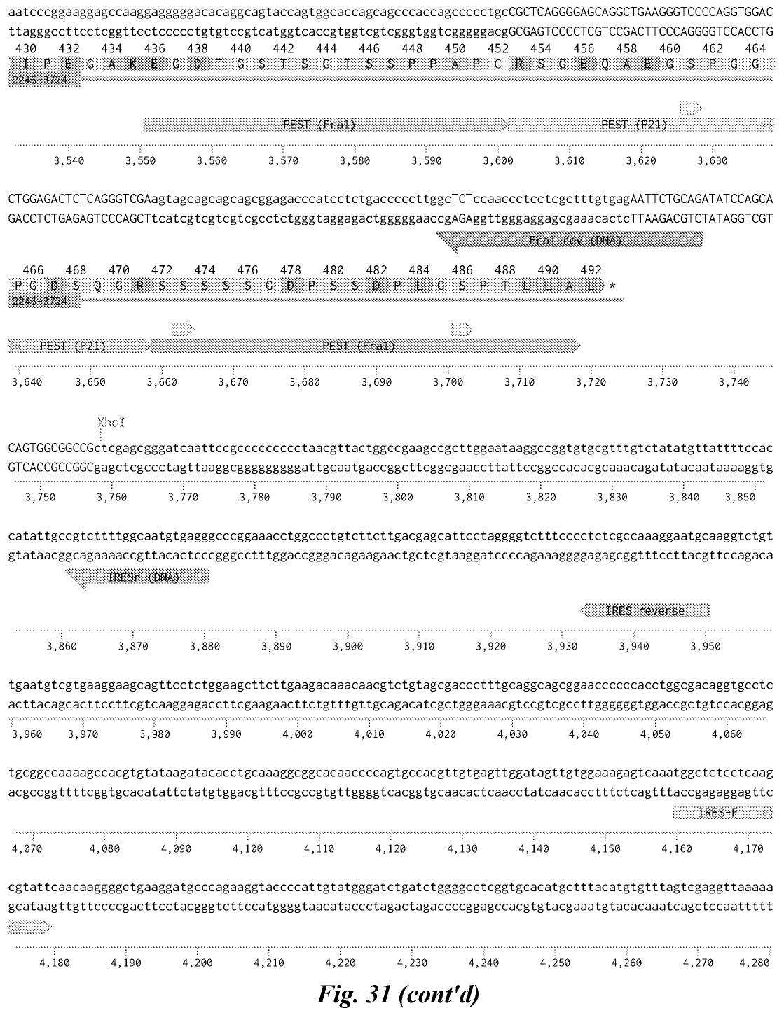

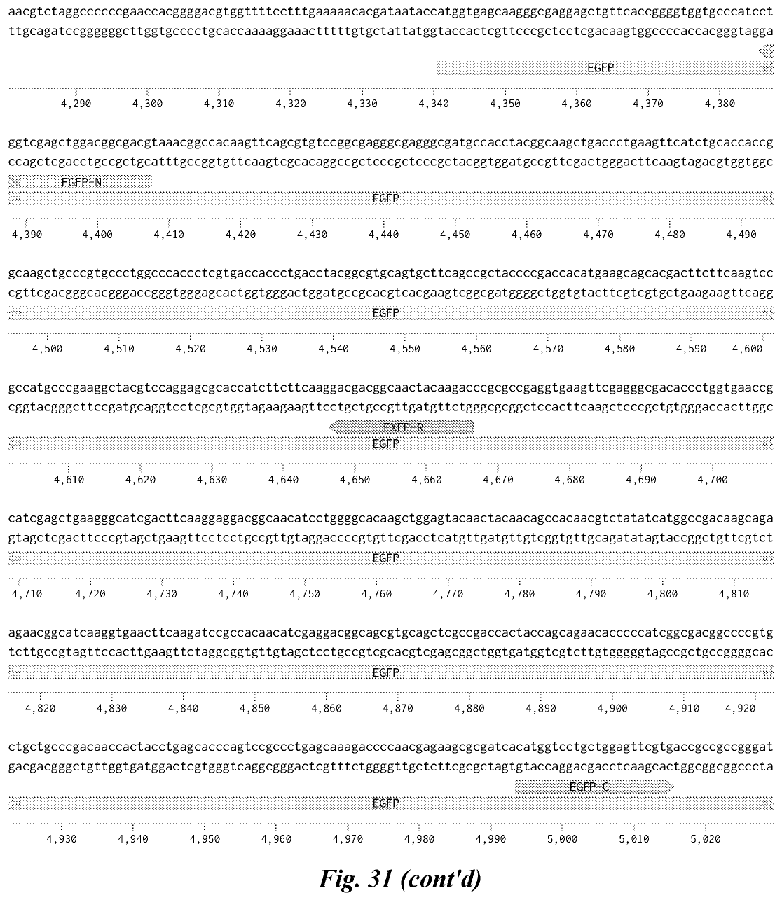

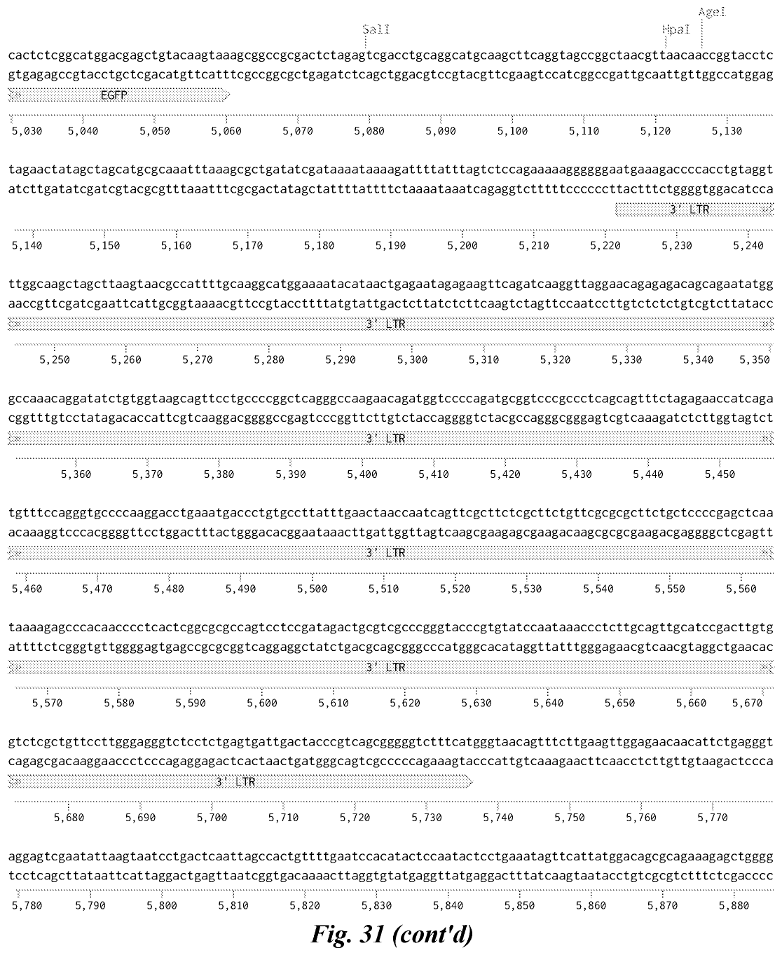

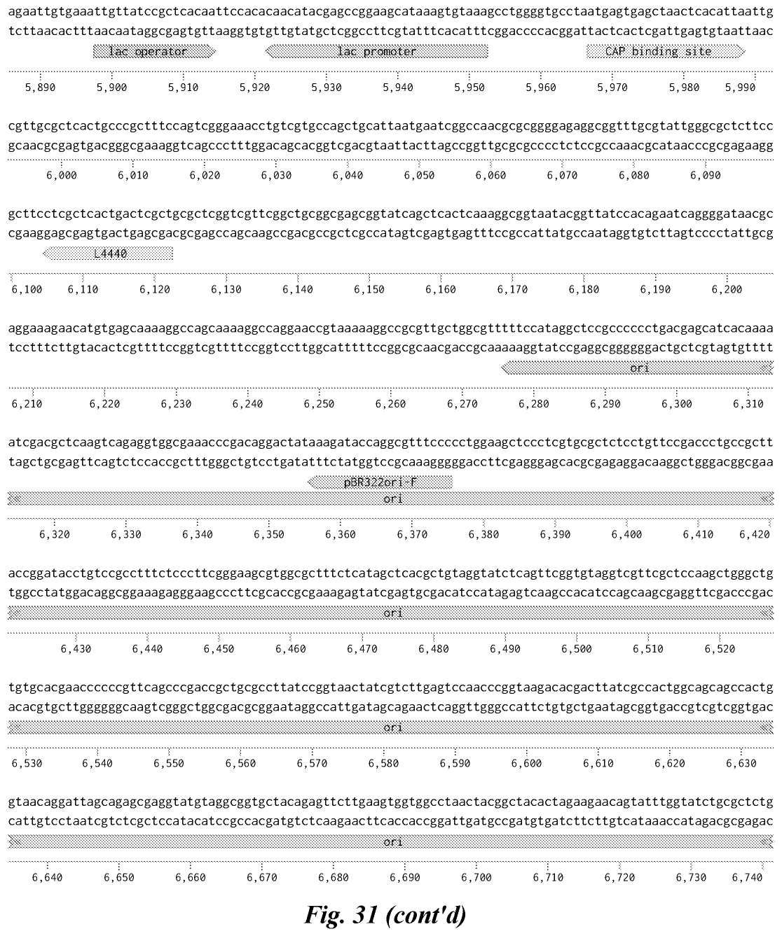

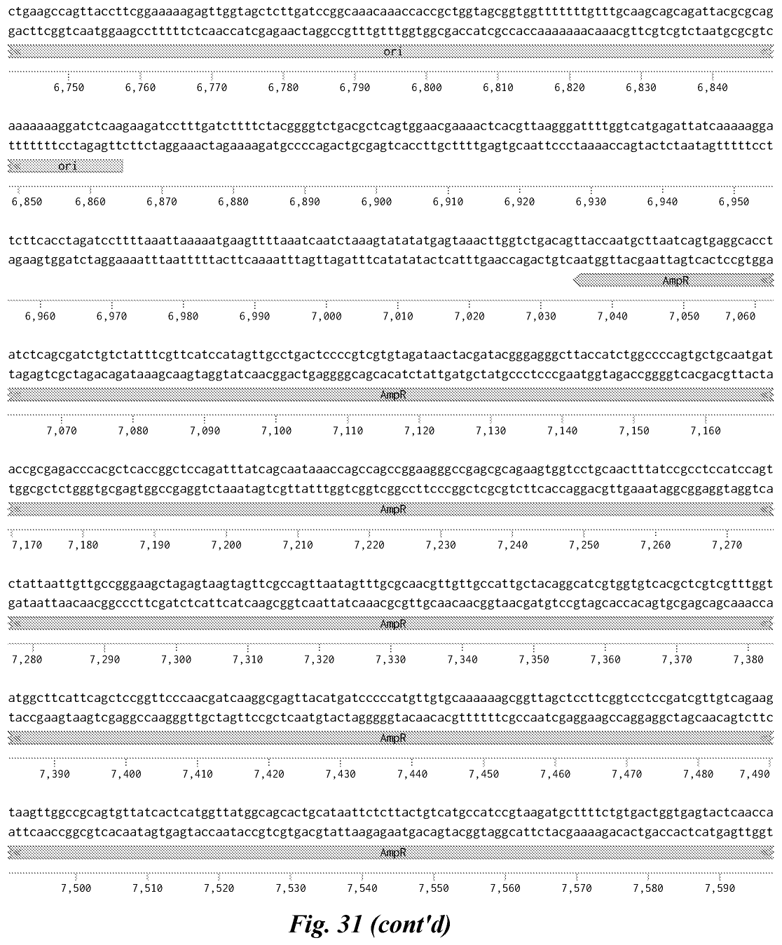

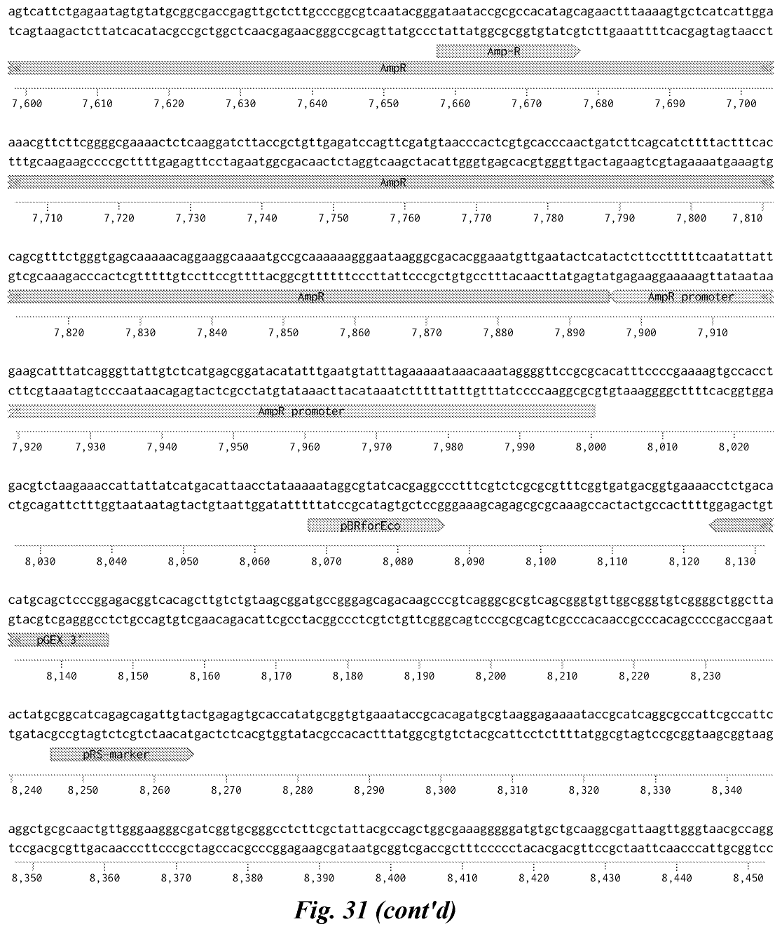

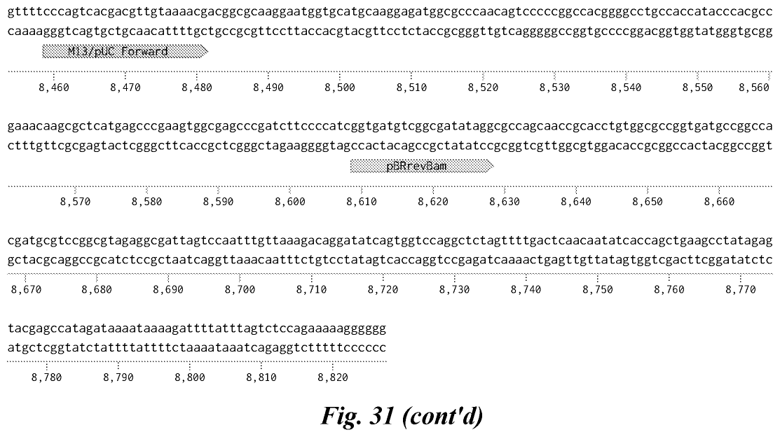

G816 and U87 cells expressing FLAG-NLS-HSVtk-CHOP were treated with temozolomide (TMZ, 100 μM) and SB203580 (p38i, 10 μM) for three days. The results are shown in FIG. 27A.

U87-FLAG-NLS-HSVtk-CHOP were treated with temozolomide (TMZ, 100 μM), SB203580 (p38i, 10 μM), or JNK-IN-8 (JNKi, 2 μM) alone or in combination for three days. The results are shown in FIG. 27B.

U87-FLAG-NLS-HSVtk-MEF2 cells were treated with trametinib (MEKi, 40 nM) or JNK-IN-8 (JNKi, 2 μM) for 24 hours. The results are shown in FIG. 27C.

### REFERENCES

All references listed herein including but not limited to all patents, patent applications and publications thereof, scientific journal articles, and database entries (e. g; GENBANK® database entries and all annotations available therein) are incorporated herein by reference in their entireties to the extent that they supplement, explain, provide a background for, or teach methodology, techniques, and/or compositions employed herein.
Albeck et al. (2013) Mol Cell 49:249-261.
Altschul et al. (1990a) J Mol Biol 215:403-410.
Altschul et al. (1990b) Proc Natl Acad Sci USA 87:14:5509-5513.
Altschul et al. (1997) Nucleic Acids Res 25:3389-3402.
Amano et al. (2011) Cancer Genomics Proteomics 8:245-250.
Bechara & Sagan (2013) FEBS Lett 587:1693-1702.
Bird et al. (1988) Science 242:423-426.
Cancer Genome Atlas Research Network (2008) Nature 455:1061-1068.
Chou & Fasman (1974) Biochemistry 13:222-245.
Chou & Fasman (1978) Ann Rev Biochem 47:251-276.
Chou & Fasman (1979) Biophys J 26:367-384.
Day et al. (2021) Molecular Therapy 29(4):1585-1601.
Deutscher et al. (ed.) (1990) Guide to Protein Purification, Harcourt Brace Jovanovich, San Diego, California, United States of America.
Devereux et al. (1984) Nucl Acids Res 12:387.
Fillat et al. (2003) Curr Gene Ther 3:13-26.
Garcia-Hernandez et al. (2021) Int J Mol Sci 23(1):370.
Genaro (1985) Remington's Pharmaceutical Sciences, Mack Publishing Co., Easton, Pennsylvania, United States of America.
Gerhardt et al. (eds.) (1994) Methods for General and Molecular Bacteriology, American Society for Microbiology, Washington, DC, United States of America. page 574.
Green et al. (2014) Molecular Cloning: A Laboratory Manual, Cold Spring Harbor Laboratory Press, Cold Spring Harbor, New York, United States of America.
Grimm et al. (1999) Gene Ther 6(7):1322-1330.
Gross & Mienhofer (1981) The Peptides, Vol. 3. Academic Press, New York, New York, United States of America.
Harlow & Lane (1988) Antibodies, A Laboratory Manual. Cold Spring Harbor Laboratory Press, Cold Spring Harbor, New York, United States of America
Harlow & Lane (1999) Using Antibodies: A Laboratory Manual. Cold Spring Harbor Laboratory Press, NY.
Houston et al. (1988) Proc Natl Acad Sci USA 85:5879-5883.
Huang et al. (2010) Protein Cell 1(3):218-226.
Karjoo et al. (2016) Adv Drug Deliv Rev 99:113-128.
Karlin & Altschul (1990) Proc Natl Acad Sci USA 87:2264-2268.
Karlin & Altschul (1993) Proc Natl Acad Sci USA 90:5873-5877.
Kim et al. (2002) Mechanisms of Signal Transduction| 277(33):P29792-29802.
Kohlbrenner et al. (2012) Hum Gene Ther Meth 23(3):198-203.
Kyte & Doolittle (1982) J Mol Biol 157:105-132.
Makkerh et al. (1996) Curr Biol 6(8):1025-1027.
Manzoor et al. (2012) J Bacter Virology 42(3):189-195.
Stupp et al. (2005) N Engl J Med 352:987-996.
U. S. Patent Application Publication Nos. 2019/0000991; 2019/0008909.
U.S. Pat. Nos. 4,554,101; 6,537,541; 10,105,451; 10,336,804.
Wang & Ron (1996) Science 272(5266):1347-1349.
Yang et al. (1999) Mol Cell Biol 19(6):4028-4038.
Zhang et al. (2014) Hum Mutation 35(9):1142-1151.
Zhao et al. (1999) Mol Cell Biol 19(1):21-30.

While the present subject matter has been disclosed with reference to specific embodiments, it is apparent that other embodiments and variations can be devised by others skilled in the art without departing from the true spirit and scope of the presently disclosed subject matter. Thus, it will be understood that various details of the presently disclosed subject matter may be changed without departing from the scope of the presently disclosed subject matter. Furthermore, the foregoing description is for the purpose of illustration only, and not for the purpose of limitation.

1. A nucleic acid construct comprising, consisting essentially of, or consisting of a first nucleic acid sequence comprising a promoter operably linked to each of a second nucleic acid sequence encoding a therapeutic polypeptide and a third nucleic acid sequence encoding a peptide domain that is stabilized when phosphorylated by kinase activity in a target cell and/or, wherein the target cell and/or is a cell and/or undergoing a stress response and the kinase activity is associated with the target cell and/or undergoing a stress response, optionally wherein the kinase is a p38 MAP kinase and/or a c-Jun N-terminal kinase (JNK).

2. The nucleic acid construct of claim 1, further comprising a fourth nucleic acid sequence encoding a nuclear localization sequence (NLS) operably linked to a promoter.

3. The nucleic acid construct of claim 1, wherein the first nucleic acid sequence comprises a promoter operably linked to a second nucleic acid sequence encoding a fusion protein comprising the therapeutic polypeptide and the peptide domain that is stabilized when phosphorylated by p38 MAP kinase activity and/or JNK activity.

4. The nucleic acid construct of claim 3, wherein the second nucleic acid sequence encoding the fusion protein comprises, consists essentially of, or consists of a first coding sequence encoding the therapeutic polypeptide, a second coding sequence encoding an NLS, and a third coding sequence encoding the peptide domain that is stabilized when phosphorylated by p38 MAP kinase activity and/or JNK activity.

5. The nucleic acid construct of claim 1, wherein the therapeutic polypeptide comprises a Herpes simplex virus thymidine kinase (HSVtk) polypeptide or a yeast cytosine deaminase polypeptide.

6. The nucleic acid construct of claim 5, wherein the HSVtk polypeptide comprises an amino acid sequence that is at least 95%, 96%, 97%, 98%, 99%, or 100% identical to SEQ ID NO: 1 or a fragment thereof.

7. (canceled)

8. The nucleic acid construct of claim 5, wherein the yeast cytosine deaminase polypeptide comprises an amino acid sequence that is at least 95%, 96%, 97%, 98%, 99%, or 100% identical to SEQ ID NO: 5 or a fragment thereof.

9. (canceled)

10. The nucleic acid construct of claim 1, wherein the peptide domain that is stabilized when phosphorylated by p38 MAP kinase activity and/or JNK activity in a target cell and/or tissue comprises, consists essentially of, or consists of a peptide domain as set forth in any of FIGS. 15-20, or a fragment thereof, or a substantially homologous amino acid sequence thereto, or a substantially homologous amino acid sequence to a fragment thereof.

11. The nucleic acid construct of claim 1, wherein the nucleic acid construct is present within a vector, optionally a viral vector.

12. The nucleic acid construct of claim 1, wherein the nucleic acid construct, optionally the vector, is disposed in a pharmaceutically acceptable diluent or vehicle.

13. (canceled)

14. (canceled)

15. A method for treating a disease or disorder in a subject in need thereof, the method comprising, consisting essentially of, or consisting of:
(a) administering to the subject a nucleic acid construct comprising, consisting essentially of, or consisting of a first nucleic acid sequence comprising a promoter operably linked to each of a second nucleic acid sequence encoding a therapeutic polypeptide and a third nucleic acid sequence encoding a peptide domain that is stabilized when phosphorylated by kinase activity in a target cell and/or, wherein the target cell and/or is a cell and/or undergoing a stress response and the kinase activity is associated with the target cell and/or undergoing a stress response, optionally wherein the kinase is a p38 MAP kinase and/or a c-Jun N-terminal kinase (JNK); and
(b) administering to the subject a prodrug that is converted by the therapeutic polypeptide to an active agent.

16. The method of claim 15, further comprising administering to the subject an additional treatment designed to treat the disease or disorder.

17. The method of claim 15, wherein the nucleic acid construct further comprises, consists essentially of, or consists of a fourth nucleic acid sequence encoding a nuclear localization sequence (NLS) operably linked to a promoter.

18. The method of claim 15, wherein the second nucleic acid sequence encodes a fusion protein comprising, consisting essentially of, or consisting of the therapeutic polypeptide and the peptide domain that is stabilized when phosphorylated by p38 MAP kinase activity and/or and/or JNK activity.

19. The method of claim 18, wherein the second nucleic acid sequence encoding the fusion protein comprises, consists essentially of, or consists of a nucleic acid sequence encoding the therapeutic polypeptide, a nucleic acid sequence encoding an NLS, and a nucleic acid sequence encoding the peptide domain that is stabilized when phosphorylated by kinase activity.

20. The method of claim 15, wherein the therapeutic polypeptide comprises, consists essentially of, or consists of a Herpes simplex virus thymidine kinase (HSVtk) polypeptide or a yeast cytosine deaminase polypeptide.

21. The method of claim 20, wherein the HSVtk polypeptide comprises, consists essentially of, or consists of an amino acid sequence that is at least 95%, 96%, 97%, 98%, 99% identical to SEQ ID NO: 1 or a fragment thereof or is 100% identical to SEQ ID NO: 1 or a fragment thereof.

22. (canceled)

23. The method of claim 20, wherein the yeast cytosine deaminase polypeptide comprises, consists essentially of, or consists of an amino acid sequence that is at least 95%, 96%, 97%, 98%, 99% identical to SEQ ID NO: 5 or a fragment thereof or is 100% identical to SEQ ID NO: 5 or a fragment thereof.

24. (canceled)

25. The method of claim 15, wherein the peptide domain that is stabilized when phosphorylated by p38 MAP kinase activity and/or JNK activity in a target cell comprises, consists essentially of, or consists of a peptide domain as set forth in any one of FIGS. 15-20, or fragment thereof, or a substantially homologous amino acid sequence thereto, or a substantially homologous amino acid sequence to a fragment thereof.

26. The method of claim 15, wherein the nucleic acid construct is present in a vector, optionally a viral vector.

27. (canceled)

28. The method of claim 15, wherein the prodrug is selected from the group consisting of ganciclovir, acyclovir, and 5-fluorocytosine.

29. The method of claim 15, wherein the disease or disorder is cancer.

30. The method of claim 29, wherein the cancer is glioblastoma.

31. The method of claim 15, further comprising administering an additional treatment and/or therapeutic agent to the subject.

32. The method of claim 31, wherein the additional treatment and/or therapeutic agent is an anti-cancer drug, radiation, surgery, or a combination thereof.

33. (canceled)

34. (canceled)


SEQUENCE LISTING (34 sequences)

SEQ ID NO: 1 - protein, 376 aa, Homo sapiens
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 2 - DNA, 1131 bp, Homo sapiens
  source 1..1131: mol_type = other DNA
atggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaactga

SEQ ID NO: 3 - DNA, 1551 bp, Homo sp.
  source 1..1551: mol_type = other DNA
cagaacccagcagccgtgtaccccgcagagccgccagccccgggcatgttccgagacttcggggaacccggcccgagctccgggaacggcggcgggtacggcggccccgcgcagcccccggccgcagcgcaggcagcccagcagaagttccacctggtgccaagcatcaacaccatgagtggcagtcaggagctgcagtggatggtacagcctcatttcctggggcccagcagttaccccaggcctctgacctaccctcagtacagccccccacaaccccggccaggagtcatccgggccctggggccgcctccaggggtacgtcgaaggccttgtgaacagatcagcccggaggaagaggagcgccgccgagtaaggcgcgagcggaacaagctggctgcggccaagtgcaggaaccggaggaaggaactgaccgacttcctgcaggcggagactgacaaactggaagatgagaaatctgggctgcagcgagagattgaggagctgcagaagcagaaggagcgcctagagctggtgctggaagcccaccgacccatctgcaaaatcccggaaggagccaaggagggggacacaggcagtaccagtggcaccagcagcccaccagccccctgccgccctgtaccttgtatctccctttccccagggcctgtgcttgaacctgaggcactgcacacccccacactcatgaccacaccctccctaactcctttcacccccagcctggtcttcacctaccccagcactcctgagccttgtgcctcagctcatcgcaagagtagcagcagcagcggagacccatcctctgacccccttggctctccaaccctcctcgctttgtgaggcgcctgagccctactccctgcagatgccaccctagccaatgtctcctccccttcccccaccggtccagctggcctggacagtatcccacatccaactccagcaacttcttctccatccctctaatgagactgaccatattgtgcttcacagtagagccagcttggggccaccaaagctgcccactgtttctcttgagctggcctctctagcacaatttgcactaaatcagagacaaaatatttcccatttgtgccagaggaatcctggcagcccagagactttgtagatccttagaggtcctctggagccctaaccccttccagatcactgccacactctccatcaccctcttcctgtgatccacccaaccctatctcctgacagaaggtgccactttacccacctagaacactaactcaccagccccactgccagcagcagcaggtgattggaccaggccattctgccgccccctcctgaaccgcacagctcaggaggcgcccttggcttctgtgatgagctgatctgcggatctcagctttgagaagccttcagctccagggaatccaagcctccacagcgagggcagctgctatttattttcctaaagagagtatttttatacaaacctaccaaaatggaataaaaggcttgaagctgtgaaaaaaaaaaaaaaaaaaaaaaaaa

SEQ ID NO: 4 - protein, 271 aa, Homo sapiens
MFRDFGEPGPSSGNGGGYGGPAQPPAAAQAAQQKFHLVPSINTMSGSQELQWMVQPHFLGPSSYPRPLTYPQYSPPQPRPGVIRALGPPPGVRRRPCEQISPEEEERRRVRRERNKLAAAKCRNRRKELTDFLQAETDKLEDEKSGLQREIEELQKQKERLELVLEAHRPICKIPEGAKEGDTGSTSGTSSPPAPCRPVPCISLSPGPVLEPEALHTPTLMTTPSLTPFTPSLVFTYPSTPEPCASAHRKSSSSSGDPSSDPLGSPTLLAL

SEQ ID NO: 5 - protein, 158 aa, Saccharomyces cerevisiae
MVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGE

SEQ ID NO: 6 - DNA, 474 bp, Saccharomyces cerevisiae
  source 1..474: mol_type = other DNA
atggtgacagggggaatggcaagcaagtgggatcagaagggtatggacattgcctatgaggaggcggccttaggttacaaagagggtggtgttcctattggcggatgtcttatcaataacaaagacggaagtgttctcggtcgtggtcacaacatgagatttcaaaagggatccgccacactacatggtgagatctccactttggaaaactgtgggagattagagggcaaagtgtacaaagataccactttgtatacgacgctgtctccatgcgacatgtgtacaggtgccatcatcatgtatggtattccacgctgtgttgtcggtgagaacgttaatttcaaaagtaagggcgagaaatatttacaaactagaggtcacgaggttgttgttgttgacgatgagaggtgtaaaaagatcatgaaacaatttatcgatgaaagacctcaggattggtttgaagatattggtgag

SEQ ID NO: 7 - protein, 8 aa, synthetic construct
MAPKKKRK

SEQ ID NO: 8 - protein, 7 aa, synthetic construct
PKKKRKV

SEQ ID NO: 9 - protein, 16 aa, synthetic construct
KRPAATKKAGQAKKKK

SEQ ID NO: 10 - protein, 9 aa, synthetic construct
PAAKRVKLD

SEQ ID NO: 11 - protein, 499 aa, Homo sapiens
MGRKKIQITRIMDERNRQVTFTKRKFGLMKKAYELSVLCDCEIALIIFNSSNKLFQYASTDMDKVLLKYTEYNEPHESRTNSDIVEALNKKEHRGCDSPDPDTSYVLTPHTEEKYKKINEEFDNMMRNHKIAPGLPPQNFSMSVTVPVTSPNALSYTNPGSSLVSPSLAASSTLTDSSMLSPPQTTLHRNVSPGAPQRPPSTGNAGGMLSTTDLTVPNGAGSSPVGNGFVNSRASPNLIGATGANSLGKVMPTKSPPPPGGGNLGMNSRKPDLRVVIPPSSKGMMPPLNTQRISSSQATQPLATPVVSVTTPSLPPQGLVYSAMPTAYNTDYSLTSADLSALQGFNSPGMLSLGQVSAWQQHHLGQAALSSLVAGGQLSQGSNLSINTNQNISIKSEPISPPRDRMTPSGFQQQQQQQQQQQPPPPPQPQPQPPQPQPRQEMGRSPVDSLSSSSSSYDGSDREDPRGDFHSPIVLGRPPNTEDRESPSVKRMRMDAWVT

SEQ ID NO: 12 - protein, 98 aa, synthetic construct
GSGGSPRMNSRKPDLRVVIPPSSKLVLEAHRPICKIPEGAKEGDTGSTSGTSSPPAPCRSPVDSLSSSSSSYDGSDRSSSSSGDPSSDPLGSPTLLAL

SEQ ID NO: 13 - protein, 169 aa, Homo sapiens
MAAESLPFSFGTLSSWELEAWYEDLQEVLSSDENGGTYVSPPGNEEEESKIFTTLDPASLAWLTEEEPEPAEVTSTSQSPHSPDSSQSSLAQEEEEEDQGRTRKRKQSGHSPARAGKQRMKEKEQENERKVAQLAEENERLKQEIERLTREVEATRRALIDRMVNLHQA

SEQ ID NO: 14 - protein, 107 aa, synthetic construct
GSGGSPRAAESLPFSFGTLSSWELEAWYEDLQEVLSSDENGGTYVSPPGNEEEESKIFTTLDPASLAWLTEEEPEPAEVTSTSQSPHSPDSSQSSLAQEEEEEDQGR

SEQ ID NO: 15 - protein, 59 aa, synthetic construct
GSGGSPRKIFTTLDPASLAWLTEEEPEPAEVTSTSQSPHSPDSSQSSLAQEEEEEDQGR

SEQ ID NO: 16 - protein, 164 aa, Homo sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 17 - protein, 81 aa, synthetic construct
GSGGSPRLVLEAHRPICKIPEGAKEGDTGSTSGTSSPPAPCRSGEQAEGSPGGPGDSQGRSSSSSGDPSSDPLGSPTLLAL

SEQ ID NO: 18 - protein, 271 aa, Homo sapiens; identical to SEQ ID NO: 4

SEQ ID NO: 19 - protein, 418 aa, Homo sapiens
MDERNRQTLRKKGLNGCESPDADDYFEHSPLSEDRFSKLNEDSDFIFKRGPPGLPPQNFSMSVTVPVTSPNALSYTNPGSSLVSPSLAASSTLTDSSMLSPPQTTLHRNVSPGAPQRPPSTGNAGGMLSTTDLTVPNGAGSSPVGNGFVNSRASPNLIGATGANSLGKVMPTKSPPPPGGGNLGMNSRKPDLRVVIPPSSKGMMPPLNTQRISSSQATQPLATPVVSVTTPSLPPQGLVYSAMPTAYNTDYSLTSADLSALQGFNSPGMLSLGQVSAWQQHHLGQAALSSLVAGGQLSQGSNLSINTNQNISIKSEPISPPRDRMTPSGFQQQQQQQQQQQPPPPPQPQPQPPQPQPRQEMGRSPVDSLSSSSSSYDGSDREDPRGDFHSPIVLGRPPNTEDRESPSVKRMRMDAWVT

SEQ ID NO: 20 - protein, 462 aa, Homo sapiens
MASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGVFKKEVYLHTSPHLKADVLFQTDPTAEMAAESLPFSFGTLSSWELEAWYEDLQEVLSSDENGGTYVSPPGNEEEESKIFTTLDPASLAWLTEEEPEPAEVTSTSQSPHSPDSSQSSLAQEEEEEDQGRTRKRKQSGHSPARAGKQRMKEKEQENERKVAQLAEENERLKQEIERLTREVEATRRALIDRMVNLHQA

SEQ ID NO: 21 - DNA, 630 bp, synthetic construct
  source 1..630: mol_type = other DNA
cgggacgccctgctgcaacttacctccgggatggtccagacccacgtcaccacccccggctccataccgacgatctgcgacctggcgcgcacgtttgcccgggagatgggggaggctaacggcagcggcggatccccgcgggcagctgagtcattgcctttctcctttgggacactgtccagctgggagctggaagcctggtatgaggacctgcaagaggtcctgtcttcagatgaaaatgggggtacctatgtttcacctcctggaaatgaagaggaagaatcaaaaatcttcaccactcttgaccctgcttctctggcttggctgactgaggaggagccagaaccagcagaggtcacaagcacctcccagagccctcactctccagattccagtcagagctccctggctcaggaggaagaggaggaagaccaagggagatgagaattcgttaacctcgagcgggatcaattccgccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcc

SEQ ID NO: 22 - protein, 147 aa, synthetic construct
RDALLQLTSGMVQTHVTTPGSIPTICDLARTFAREMGEANGSGGSPRAAESLPFSFGTLSSWELEAWYEDLQEVLSSDENGGTYVSPPGNEEEESKIFTTLDPASLAWLTEEEPEPAEVTSTSQSPHSPDSSQSSLAQEEEEEDQGR

SEQ ID NO: 23 - DNA, 422 bp, synthetic construct
  source 1..422: mol_type = other DNA
cgggacgccctgctgcaacttacctccgggatggtccagacccacgtcaccacccccggctccataccgacgatctgcgacctggcgcgcacgtttgcccgggagatgggggaggctaacggcagcggcggatccccgcggatgaacagtaggaaaccagaccttcgagttgtcatccccccttcaagcaagctggtgctggaagcccaccgacccatctgcaaaatcccggaaggagccaaggagggggacacaggcagtaccagtggcaccagcagcccaccagccccctgccgctcccctgtggacagtctgagcagctctagtagctcctatgatggcagtgatcggagtagcagcagcagcggagacccatcctctgacccccttggctctccaaccctcctcgctttgtgagaatt

SEQ ID NO: 24 - protein, 138 aa, synthetic construct
RDALLQLTSGMVQTHVTTPGSIPTICDLARTFAREMGEANGSGGSPRMNSRKPDLRVVIPPSSKLVLEAHRPICKIPEGAKEGDTGSTSGTSSPPAPCRSPVDSLSSSSSSYDGSDRSSSSSGDPSSDPLGSPTLLAL

SEQ ID NO: 25 - DNA, 422 bp, synthetic construct
  source 1..422: mol_type = other DNA
cgggacgccctgctgcaacttacctccgggatggtccagacccacgtcaccacccccggctccataccgacgatctgcgacctggcgcgcacgtttgcccgggagatgggggaggctaacggcagcggcggatccccgcggctggtgctggaagcccaccgacccatctgcaaaatcccggaaggagccaaggagggggacacaggcagtaccagtggcaccagcagcccaccagccccctgccgctcaggggagcaggctgaagggtccccaggtggacctggagactctcagggtcgaagtagcagcagcagcggagacccatcctctgacccccttggctctccaaccctcctcgctttgtgagaattcgttaacctcgagcgggatcaattccgccccccccctaacgttactggccg

SEQ ID NO: 26 - protein, 121 aa, synthetic construct
RDALLQLTSGMVQTHVTTPGSIPTICDLARTFAREMGEANGSGGSPRLVLEAHRPICKIPEGAKEGDTGSTSGTSSPPAPCRSGEQAEGSPGGPGDSQGRSSSSSGDPSSDPLGSPTLLAL

SEQ ID NO: 27 - DNA, 8905 bp, synthetic construct
  source 1..8905: mol_type = other DNA
aatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattcagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattaaccatggactacaaagaccatgacggtgattataaagatcatgacatcgattacaaggatgacgatgacaagcttggtaccatggcccccaagaagaagaggaaggtgatggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccacccccggctccataccgacgatctgcgacctggcgcgcacgtttgcccgggagatgggggaggctaacggcagcggcggatccccgcgggcagctgagtcattgcctttctcctttgggacactgtccagctgggagctggaagcctggtatgaggacctgcaagaggtcctgtcttcagatgaaaatgggggtacctatgtttcacctcctggaaatgaagaggaagaatcaaaaatcttcaccactcttgaccctgcttctctggcttggctgactgaggaggagccagaaccagcagaggtcacaagcacctcccagagccctcactctccagattccagtcagagctccctggctcaggaggaagaggaggaagaccaagggagatgagaattctgcagatatccagcacagtggcggccgctcgagcgggatcaattccgccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcgacctgcaggcatgcaagcttcaggtagccggctaacgttaacaaccggtacctctagaactatagctagcatgcgcaaatttaaagcgctgatatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaagggggg

SEQ ID NO: 28 - protein, 518 aa, synthetic construct
MDYKDHDGDYKDHDIDYKDDDDKLGTMAPKKKRKVMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEANGSGGSPRAAESLPFSFGTLSSWELEAWYEDLQEVLSSDENGGTYVSPPGNEEEESKIFTTLDPASLAWLTEEEPEPAEVTSTSQSPHSPDSSQSSLAQEEEEEDQGR

SEQ ID NO: 29 - DNA, 8761 bp, synthetic construct
  source 1..8761: mol_type = other DNA
aatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattcagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattaaccatggactacaaagaccatgacggtgattataaagatcatgacatcgattacaaggatgacgatgacaagcttggtaccatggcccccaagaagaagaggaaggtgatggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccacccccggctccataccgacgatctgcgacctggcgcgcacgtttgcccgggagatgggggaggctaacggcagcggcggatccccgcggaaaatcttcaccactcttgaccctgcttctctggcttggctgactgaggaggagccagaaccagcagaggtcacaagcacctcccagagccctcactctccagattccagtcagagctccctggctcaggaggaagaggaggaagaccaagggagatgagaattctgcagatatccagcacagtggcggccgctcgagcgggatcaattccgccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcgacctgcaggcatgcaagcttcaggtagccggctaacgttaacaaccggtacctctagaactatagctagcatgcgcaaatttaaagcgctgatatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaagggggg

SEQ ID NO: 30 - protein, 470 aa, synthetic construct
MDYKDHDGDYKDHDIDYKDDDDKLGTMAPKKKRKVMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEANGSGGSPRKIFTTLDPASLAWLTEEEPEPAEVTSTSQSPHSPDSSQSSLAQEEEEEDQGR

SEQ ID NO: 31 - DNA, 8878 bp, synthetic construct
  source 1..8878: mol_type = other DNA
aatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattaaccatggactacaaagaccatgacggtgattataaagatcatgacatcgattacaaggatgacgatgacaagcttggtaccatggcccccaagaagaagaggaaggtgatggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccacccccggctccataccgacgatctgcgacctggcgcgcacgtttgcccgggagatgggggaggctaacggcagcggcggatccccgcggatgaacagtaggaaaccagaccttcgagttgtcatccccccttcaagcaagctggtgctggaagcccaccgacccatctgcaaaatcccggaaggagccaaggagggggacacaggcagtaccagtggcaccagcagcccaccagccccctgccgctcccctgtggacagtctgagcagctctagtagctcctatgatggcagtgatcggagtagcagcagcagcggagacccatcctctgacccccttggctctccaaccctcctcgctttgtgagaattctgcagatatccagcacagtggcggccgctcgagcgggatcaattccgccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattcagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcgacctgcaggcatgcaagcttcaggtagccggctaacgttaacaaccggtacctctagaactatagctagcatgcgcaaatttaaagcgctgatatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaagggggg

SEQ ID NO: 32 - protein, 509 aa, synthetic construct
MDYKDHDGDYKDHDIDYKDDDDKLGTMAPKKKRKVMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEANGSGGSPRMNSRKPDLRVVIPPSSKLVLEAHRPICKIPEGAKEGDTGSTSGTSSPPAPCRSPVDSLSSSSSSYDGSDRSSSSSGDPSSDPLGSPTLLAL

SEQ ID NO: 33 - DNA, 8827 bp, synthetic construct
  source 1..8827: mol_type = other DNA
aatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattcagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattaaccatggactacaaagaccatgacggtgattataaagatcatgacatcgattacaaggatgacgatgacaagcttggtaccatggcccccaagaagaagaggaaggtgatggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgccccgacgatctgcgacctggcgcgcacgtttgcccgggagatgggggaggctaacggcagcggcggatccccgcggctggtgctggaagcccaccgacccatctgcaaaatcccggaaggagccaaggagggggacacaggcagtaccagtggcaccagcagcccaccagccccctgccgctcaggggagcaggctgaagggtccccaggtggacctggagactctcagggtcgaagtagcagcagcagcggagacccatcctctgacccccttggctctccaaccctcctcgctttgtgagaattctgcagatatccagcacagtggcggccgctcgagcgggatcaattccgccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcgacctgcaggcatgcaagcttcaggtagccggctaacgttaacaaccggtacctctagaactatagctagcatgcgcaaatttaaagcgctgatatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaaagagctggggagaattgtgaaattgttatccgctcacaattccacacaacatacgagccggaagcataaagtgtaaagcctggggtgcctaatgagtgagctaactcacattaattgcgccggctgccgggacgccctgctgcaacttacctccgggatggtccagacccacgtcaccacccccggctccatacgttgcgctcactgcccgctttccagtcgggaaacctgtcgtgccagctgcattgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggataatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaagggggg

SEQ ID NO: 34 - protein, 467 aa, synthetic construct
MDYKDHDGDYKDHDIDYKDDDDKLGTMAPKKKRKVMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPRRSATWRARLPGRWGRLTAAADPRGWCWKPTDPSAKSRKEPRRGTQAVPVAPAAHQPPAAQGSRLKGPQVDLETLRVEVAAAAETHPLTPLALQPSSLC